(19) United States
(12) Patent Application Publication
(10) Pub. No.: US 2011/0190380 A1
(43) Pub. Date: Aug. 4, 2011

(54) METHODS FOR DELIVERY OF SIRNA TO BONE MARROW CELLS AND USES THEREOF

(76) Inventors: Elena Feinstein, Rehovot (IL); Hagar Kalinski, Rishon-le-Zion (IL); Igor Mett, Rehovot (IL); Hagit Ashush, Ashkelon (IL)

(21) Appl. No.: 13/061,934

(22) PCT Filed: Oct. 23, 2008
(86) PCT No.: PCT/IL08/01401

§ 371 Date: Apr. 18, 2011

(51) Int. Cl. A61K 31/7105; A61K 31/7088; C07H 21/00; A61P 35/00

(52) U.S. Cl. 514/44 A; 536/24.5

(57) ABSTRACT

The present invention relates to a method for the delivery of therapeutic oligonucleotides to bone marrow, and in particular delivery of siRNA to a subset of bone marrow cells. The method comprises systemically administering siRNA to a subject in need thereof, to reduce or inhibit expression of a gene associated with a disease or disorder or to symptoms associated with a disease or disorder associated with the cells. The invention further relates to chemically modified siRNA compounds, to pharmaceutical compositions comprising such compounds and to methods of using such compounds and compositions in the treatment of disease.

### FIELD OF THE INVENTION

The present invention relates to a method for the delivery of therapeutic oligonucleotides to bone marrow, and in particular delivery of siRNA to a subset of bone marrow cells. The method comprises systemically administering siRNA to a subject in need thereof, to reduce or inhibit expression of a gene associated with a disease or disorder or to symptoms associated with a disease or disorder.

### BACKGROUND OF THE INVENTION

### Bone Marrow Cells and Tumorigenesis

Bone marrow is a specialized tissue that produces a plurality of different cell types, including stromal cells, hematopoietic lineage cells and cells involved in bone remodeling. Bone marrow is the primary source of red blood cells (erythrocytes) and White blood cells in the body. Hematopoiesis is an ongoing process whereby highly specialized blood cells are generated from hematopoietic stem cells (HSC). The specialized cells fall within two functionally distinct groups termed myeloid and lymphoid cells.

Cells of the lymphoid lineage, which develop into B-cells (lymphocytes) or T-cells (Th or CTL), are produced to varying degrees in the bone marrow, spleen, thymus and lymph nodes. The principle function of B-cells lies in the production of antibodies.

During normal human adult life, myeloid cells are produced exclusively within the bone marrow. Myeloid stem cells produce the progenitor cells for the neutrophil, monocyte, macrophage, eosinophil, erythrocyte, megakaryocyte, mast cell, platelet and basophil cell types.

Myeloid lineage hematopoietic cells were shown to stimulate angiogenesis either directly by secreting angiogenic factors or indirectly by producing extracellular matrix-degrading proteases, which in turn release sequestered angiogenic factors (reviewed in Lewis & Pollard. Cancer Res. 2006. 66:605-612; Naldini & Carraro Curr Drug Targets Inflamm Allergy 2005. 4:3-8). Furthermore, Gr1+/CD11b+ progenitor cells isolated from the spleens of tumor-bearing mice promoted angiogenesis when co-injected with tumor cells (Yang, et al. Cancer Cell 2004. 6:409-21). Implantation of tumor cells in mice resulted in upregulation of Bv8 (prokinectin 2; PROK2), mobilization of Gr1+/CD11b+ myeloid cells from the bone marrow and promotion of angiogenesis. An anti-Bv8 antibody suppressed angiogenesis and reduced Gr1+/CD11b+ myeloid cells following implantation of tumor cells in mice. (Shajaei et al, Nature 2007. 450(7171):825-31).

Myeloid cells accumulating in tumor-bearing hosts play an important role in tumor non-responsiveness by suppressing antigen-specific T cell responses (Almand et al., J. Immunol. 2001. 166:678-689; Bronte et al., J. Immunoth. 2001. 24:431-446; Gabrilovich, Nat Rev Immunol. 2004. 4:941-952; Kusmartsev et al., J. Immunol. 2000.165:779-785; Melani et al., Blood. 2003.102(6):2138-45; Pandit et al., Ann Otol Rhinol Laryngol. 2000. 109:749-754). These cells contribute to the failure of immune therapy in patients with advanced cancer and in tumor-bearing mice. In mice, the myeloid cells are characterized as Gr1+/CD11b+ cells. Morphological analysis demonstrated that they are comprised of a mixture of myeloid cells, such as granulocytes and monocyte-macrophages, as well as myeloid cell precursors at various stages of differentiation. Since they display features of undifferentiated myeloid cells and contain precursors of different myeloid cell subsets, these cells have been termed “immature myeloid cells” (ImC) or myeloid derived suppressive cells (Gabrilovich, et al, Cancer Res. 2007, 67:425). The equivalent cells in human are CD33+CD11b+ cells.

ImC cells represent about 20-30% of normal bone marrow cells and only 2-4% of all nucleated normal splenocytes. Inoculation of transplantable tumor cells (Kusmartsev et al. supra; Bronte et al., J. Immunol. 1999. 162:5728-37; Gabrilovich et al., J. Immunol. 2001. 166:5398-5406; Subiza et al., Int J. Cancer. 1989, 44:307-314) or spontaneous development of tumors in transgenic mice with tissue-restricted expression of oncogenes (Melani, supra) results in a marked systemic expansion of these cells. The proportion of this myeloid cell population in spleen of tumor-bearing mice may reach up to 50% of all splenocytes (Kusmartsev & Gabrilovich, J Leukoc Biol. 2003, 74(2):186-196). In cancer patients, advanced-stage cancer of head and neck, lung or breast cancers were found to promote the accumulation of these cells in the peripheral blood, whereas surgical resection of the tumors decreased the number of ImC (Almand et al., Clin Cancer Res. 2000, 6:1755-1766).

The accumulation of Gr1+/CD11b+ myeloid cells is often associated with a large tumor burden and a state of immune suppression (Bronte et al., Blood. 2000, 96:383812; Gabrilovich et al., 2001, supra; Kusmartsev et al, 2000 supra; Melani et al., 2003 supra; Subiza et al., Int J. Cancer. 1989, 44:307-31).

ImC derived from tumor bearing mice have been shown to
i) Induce loss or significant decrease of the expression of the T cell receptor ζ chain (CD3ζ CD3 zeta), which is the principal part of TCR complex (Otsuji et al., PNAS USA. 1996, 93:13119-1312);
ii) Inhibit CD3/CD28-induced T cell activation/proliferation by production of reactive nitrogen and oxygen intermediates (Kusmartsev et al., 2000, supra);
iii) Inhibit interferon-γ (IFN-γ) production by CD8+T cells in response to the specific peptide presented by MHC class I molecules (Gabrilovich et al., 2001 supra);
iv) Regulate tumor vascularization and tumor progression by MMP-9 (Yang et al., Cancer Cell. 2004, 6(4):409-21);
v) Suppress T cell proliferation by producing TGFβ (Beck et al., Eur J Immunol. 2001, 33(1):19-28; Terabe et al., J Exp Med. 2003, 198(11):1741-52; Young et al., J. Immunol. 1996, 156:1916-1921);
vi) Lead to T cell unresponsiveness by STAT1 (Shankaran et al., Nature. 2001, 410:1107-111).

Gu et al., (Scan, J. Immunol. 2006, 64:588-594) demonstrated in vitro RNAi for CD80 and CD86 in dendritic cells. Suzuki et al (J of Immunol. 2007.174:88.4) showed immune modulation through in vitro siRNA silencing of CD80 and CD86 in dendritic cells. Terabe et al., (J Exp Med. 2003, 198(11):1741-52) demonstrated that depletion of Gr1+ myeloid cells or blockage of TGFβ in vivo prevented tumor recurrence.

US Patent Application Publication No. 2007/0264193 is directed to combination therapy for the treatment of resistant tumors.

### NADPH Oxidase

The NADPH oxidase (NOX) family of proteins in humans consists of at least thirteen unique gene products: NOX1, NOX2 (gp91phox, CYBB), NOX3, NOX4, NOX5, DUOX1 and DUOX2 and associated proteins p22phox (CYBA), NOXO1, NOXO2 (p47phox, NCF1) NOXA1, NOXA2 (p67phox, NCF2) and p40phox (NCF4) (hereinafter “NOX genes). Reactive oxygen species (ROS) generated in many tissues has been shown to originate from the activity of NOX enzymes and NOX gene expression has been associated with various pathological processes (comprehensive review in Bedard and Krause, Physiol. Rev. 2007. 87:245-313,).

International Patent Publication No. WO 2005/119251 discloses a method of inhibiting NOX3 for treating hearing loss. International Patent Publication No. WO 2002/030453 discloses NADPH oxidase inhibitors for reducing angiogenesis. U.S. Pat. No. 6,846,672 and related patents and patent applications disclose the polynucleotide and polypeptide sequences of the NOX enzymes. US Patent Publication No. 2007/0037883 relates to NOX4 inhibition. U.S. Pat. Nos. 6,846,672; 7,029,673; 7,202,052; 7,202,053 and 7,226,769 disclose NOX enzymes and regulators thereof.

siRNAs and RNA Interference

RNA interference (RNAi) is a phenomenon involving double-stranded (ds) RNA-dependent gene specific posttranscriptional silencing. Originally, attempts to study this phenomenon and to manipulate mammalian cells experimentally were frustrated by an active, non-specific antiviral defense mechanism which was activated in response to long dsRNA molecules (Gil et al. Apoptosis, 2000. 5:107-114). Later it was discovered that synthetic duplexes of 21 nucleotide RNAs could mediate gene specific RNAi in mammalian cells, without the stimulation of the generic antiviral defense mechanisms (see Elbashir et al. Nature 2001, 411:494-498 and Caplen et al. PNAS USA 2001, 98:9742-9747). As a result, small interfering RNAs (siRNAs), which are short double-stranded RNAs, have become powerful tools in attempting to understand gene function. Thus RNA interference (RNAi) refers to the process of sequence-specific post-transcriptional gene silencing in mammals mediated by small interfering RNAs (siRNAs) (Fire et al, Nature 1998. 391, 806) or microRNAs (miRNA; Ambros, Nature 2004 431:7006, 350-55; and Bartel, Cell. 2004. 116(2):281-97). The corresponding process in plants is commonly referred to as specific post transcriptional gene silencing or RNA silencing and is referred to as quelling in fungi.

An siRNA is a double-stranded RNA molecule which inhibits, either partially or fully, the expression of a gene/mRNA of its endogenous or cellular counterpart, or of an exogenous gene such as a viral nucleic acid. The mechanism of RNA interference is detailed infra.

siRNA has recently been successfully used for inhibition in primates (Tolentino et al., Retina 2004. 24(1):132-138). Several studies have revealed that siRNA therapeutics are effective in vivo in both mammals and in humans. Bitko et al., have shown that specific siRNA molecules directed against the respiratory syncytial virus (RSV) nucleocapsid N gene are effective in treating mice when administered intranasally (Bitko et al., Nat. Med. 2005, 11(1):50-55). For a review of the use of siRNA as therapeutics, see Barik (J. Mol. Med. 2005. 83: 764-773).

None of the above references teaches a method for the targeted delivery of siRNA to the bone marrow or to a particular subset of bone marrow cells. There remains a yet unmet need for safe, targeted and efficient tumor therapy and treatment for transplant rejection.

### SUMMARY OF THE INVENTION

The present invention is based in part on the unexpected finding that siRNA compounds can be targeted to the bone marrow, and in particular to a subset of bone marrow cells. The high specificity of the siRNA to its target cells affords an effective method for delivery of specific siRNA to those cells and attendant inhibition of target genes in the cells.

Accordingly, in one aspect the present invention provides a method of treating a bone marrow disorder in a subject in need thereof, which comprises systemically administering to the subject an oligonucleotide which inhibits expression of a target gene associated with the disorder in bone marrow cells of the subject in an amount effective to treat the disorder. In some embodiments the bone marrow cells are immature myeloid cells. In certain embodiments the immature myeloid cells are CD11b+, and preferably CD33+/CD11b+. In various embodiments the oligonucleotide comprises a sufficient number of consecutive nucleotides having a sequence of sufficient homology to a nucleic acid sequence present within the gene to hybridize to the gene and reduce or inhibit expression of the gene in the subject.

In one aspect the present invention provides a method of treating a disorder associated with immature myeloid cell expansion and or mobilization in a subject in need of such treatment which comprises systemically administering to the subject a therapeutically effective amount of an siRNA directed to a target gene associated with the disorder in an amount effective to treat the subject.

In another aspect the present invention provides a method of treating cancer in a subject in need of which comprises systemically administering to the subject a therapeutically effective amount of an oligonucleotide which inhibits expression of a target gene expressed in an immature myeloid cell in the subject in an amount effective to treat the subject. In certain embodiments the target gene is selected from a gene whose mRNA is listed in Table A1 and set forth in SEQ ID NOS:1-87. In certain embodiments the gene is selected from ARG1, MMP9, PROK2, NOS2A, TGFβ1, CD80 and STAT1.

In yet another aspect the present invention provides method of reducing immature myeloid cell expansion or mobilization in a subject in need thereof which comprises systemically administering to the subject a therapeutically effective amount of an oligonucleotide which inhibits expression of a gene expressed in the immature myeloid cell in the subject in an amount effective to reduce immature myeloid cell expansion or mobilization.

In yet another aspect the present invention provides method of treating a subject suffering from a disorder associated with immature myeloid cell expansion or immature myeloid cell mobilization which comprises systemically administering to the subject a therapeutically effective amount of an oligonucleotide which inhibits expression of a gene expressed in the immature myeloid cell in the subject in an amount effective to reduce immature myeloid cell expansion or mobilization.

In some embodiments of the methods set forth above the disorder associated with immature myeloid cell expansion and or mobilization is selected from tumorigenesis, tumor progression, tumor neoangiogenesis, tumor resistance, and allograft rejection. In some embodiments the tumor is a solid tumor or a hematopoietic tumor of a myeloid lineage. The tumor can be inter alia head and neck, breast, lung, kidney, prostate, colon or a pancreatic tumor. In some embodiments inhibition of gene expression results in reduced tumor load. The oligonucleotide of the present invention can be administered alone, in combination with one or more additional oligonucleotides and/or in combination with a chemotherapeutic agent. The oligonucleotide is preferable an siRNA comprising a sufficient number of consecutive nucleotides having a sequence of sufficient homology to a nucleic acid sequence present within the gene to hybridize to its corresponding mRNA and reduce or inhibit expression of the target gene in the subject. In other aspects the present invention provides a method of treating a subject suffering from a disorder in which the level of T cell receptor ζ chain (CD3ζ; CD3 zeta) is reduced or absent which comprises systemically administering to the subject an oligonucleotide which inhibits expression of a gene expressed in an immature myeloid cell in the subject in an amount effective to treat the subject.

Yet in other aspects the present invention provides a method of reducing tumor vascularization and tumor progression in a subject in need thereof which comprises systemically administering to the subject a therapeutically effective amount of an oligonucleotide which inhibits expression of a gene expressed in an immature myeloid cell in the subject in an amount effective to reduce tumor vascularization and tumor progression.

The present invention further provides a method of preventing allograft transplant rejection in a subject in need thereof which comprises systemically administering to the subject a therapeutically effective amount of an oligonucleotide which inhibits expression of a gene expressed in an immature myeloid cell in the subject in an amount effective to treat the transplant rejection. In certain embodiments the gene is CD80 or CD86.

In yet another aspect the present invention provides a method of delivering an oligonucleotide to a CD11b+ immature myeloid cell in a subject in need thereof which comprises administering systemically to the subject a therapeutically effective amount of an oligonucleotide which inhibits expression of a gene expressed in the immature myeloid cell in the subject in an amount effective to achieve delivery to the CD11b+ immature myeloid cell.

In various embodiments of the methods set forth above, the immature myeloid cell is CD33+/CD11b+.

In certain embodiments of the methods set forth above the oligonucleotide is siRNA.

In various embodiments of the methods set forth above the siRNA is naked siRNA.

In various preferred embodiments the siRNA is chemically modified siRNA.

In various embodiments the siRNA has structure (A) set forth below:

| | | | | |
|---|---|---|---|---|
| (A) | 5′ | (N)ₓ-Z | 3′ | (antisense strand) |
| | 3′ | Z′-(N′)_{y} | 5′ | (sense strand) |

wherein each of N and N′ is a nucleotide selected from an unmodified ribonucleotide, a modified ribonucleotide, an unmodified deoxyribonucleotide and a modified deoxyribonucleotide;
wherein each of (N)ₓ and (N′)_{y} is an oligonucleotide in which each consecutive N or N′ is joined to the next N or N′ by a covalent bond;
wherein each of x and y is an integer between 18 and 40;
wherein each of Z and Z′ may be present or absent, but if present is 1-5 consecutive nucleotides covalently attached at the 3′ terminus of the strand in which it is present; and
wherein the sequence of (N′)_{y} is present within an mRNA expressed in an immature myeloid cell.

In certain embodiments in (N)x the nucleotides alternate between modified ribonucleotides and unmodified ribonucleotides each modified ribonucleotide being modified so as to have a 2′-O-methyl on its sugar and the ribonucleotide located at the middle position of (N)x being unmodified and the ribonucleotide located at the middle; and wherein the oligonucleotide sequence of (N)x is complementary to the oligonucleotide sequence of (N′).

In various embodiments (N′)y comprises unmodified ribonucleotides in which one nucleotide at a terminal or penultimate position is modified wherein the modified nucleotide is selected from the group consisting of a mirror nucleotide, a bicyclic nucleotide, a 2′-sugar modified nucleotide, an altriol nucleotide or a nucleotide joined to an adjacent nucleotide by a 2′-5′ phosphodiester bond; and wherein if more than one nucleotide is modified in (N′)y, the modified nucleotides are consecutive.

In other embodiments in (N′)y the nucleotides alternate between modified ribonucleotides and unmodified ribonucleotides each modified ribonucleotide being modified so as to have a 2′-O-methyl on its sugar and the ribonucleotide located at the middle position of (N)x being unmodified and the ribonucleotide located at the middle; and wherein the oligonucleotide sequence of (N)x is complementary to the oligonucleotide sequence of (N′).

Preferably Z and Z′ are absent. In certain embodiments x=y=19 or x=y=23. Preferably the oligonucleotide sequence of (N)x is complementary to the oligonucleotide sequence of (N′)y. In other embodiments 1, 2 or 3 mismatches between the sequences of (N)x and (N′)y are allowed.

In various embodiments of the methods set forth above the mRNA is listed in Tables A1 and A2 and is set forth in SEQ ID NOS: 1-89. In certain preferred embodiments the sequence of (N)ₓ comprises one or more of the antisense sequences present in Tables B (B1-B25; SEQ ID NOS:90-24,075) and Table G (SEQ ID NOS:24,076-24,117).

In another aspect the present invention provides chemically and or structurally modified siRNA compounds based on Structures (C)-(P) disclosed herein. In various embodiments the siRNA compounds target mRNA set forth in Tables A1 and A2. In various embodiments the oligonucleotide sequence of (N)x is set forth in any one of Tables B (B1-B25; SEQ ID NOS:90-24,075) and Table G (SEQ ID NOS:24,076-24,117).

In another aspect the present invention provides a pharmaceutical composition comprising a compound according to the invention; and a pharmaceutically acceptable carrier.

The present invention provides novel oligonucleotide sequences useful in inhibiting a target gene set forth in Tables A1 and A2, and to methods of use thereof. In some embodiments the oligonucleotide is selected from the group consisting of an antisense oligonucleotide, shRNA, siRNA, a ribozyme, miRNA. In certain preferred embodiment the oligonucleotide is siRNA. Tables B1-B17 show 19-mer sense and corresponding antisense oligonucleotides of the present invention. Tables B18-B25 show 19-, 21- and 23-mer compounds for CD80 and CD86. Table G shows certain 19-mer sense and corresponding antisense oligonucleotides directed to various target genes.

In another aspect the present invention provides chemically and or structurally modified nucleic acid compounds useful in inhibiting expression of a gene selected from the group consisting of CD80, CD86, MMP9, PROK2, NOS2A, ARG1, TGFβ2, STAT1, STAT3, STAT6, RAC1, RAC2, NOX1, NOX2, NOX3, NOX4, NOX5, DUOX1, DUOX2, NOXO1, NOXO2 (p47phox, NCF1), NOXA1, NOXA2 (p67phox, NCF2), CYBA, ELA2, Expi, LDLR, TLR-1, RLF, FGF13, IL-4R, IL-11R, IL-13R, IL-1R2, IL-10, IFN, TNFRSF18, WNT5A, SCAMP1, HSP86, EGFR, EphA5, EphRB2, Eph-RA7, HGF, ANGPTL6, NTF5, CLDN18, MDC15, MMRN1, CD11b, CD14, CD18, CD29 (ITGB1), CD120a, CD120b, Ep-1 (PTGER1), PEX-5, CD33, REG3A, PGK1, ILRN1, CASP2 and HIF1a.

Novel structures of double stranded oligonucleotides, having advantageous properties and which may be applied to siRNA to any of the above mentioned target sequences, and in particular to the siRNA oligonucleotides disclosed herein. The present invention also provides pharmaceutical compositions comprising one or more such oligonucleotides or a vector capable of expressing the oligonucleotide. The present invention further relates to methods for treating or preventing the incidence or severity of various diseases or conditions in a subject in need thereof wherein the disease or condition and/or symptoms associated therewith. The diseases and disorders are associated with immature myeloid cell expansion and or mobilization. Such methods involve administering to a mammal in need of such treatment a prophylactically or therapeutically effective amount of one or more such compounds, which inhibit or reduce expression or activity of at least one such gene.

In another aspect, the present invention relates to a method for the treatment of a subject in need of treatment for a disease or disorder or symptoms associated with the disease or disorder, associated with the expression of a gene set forth in Tables A1 and A2 comprising administering to the subject an amount of an siRNA, according to the present invention, in a therapeutically effective dose so as to thereby treat the subject. More specifically, the present invention provides methods and compositions useful in treating a subject suffering from cancer or transplant rejection. The present invention further relates to the use of an oligonucleotide compound for promoting recovery from a disease associated with immature myeloid cell expansion and or cell immobilization. Additionally the present invention relates to the use of an oligonucleotide compound for the preparation useful in treating a disease associated with immature myeloid cell expansion and or cell immobilization.

Known modified siRNA compounds are explicitly excluded from the present invention.

### BRIEF DESCRIPTION OF THE FIGURES

FIG. 1: siRNA distribution in various tissues following intravenous administration to normal and 5/6 nephrectomized rats

FIG. 2: siRNA activity in rat bone marrow cells 24, 48 and 72 hrs following single bolus intravenous injection.

FIG. 3: Cy3-siRNA distribution as determined by FACS.

FIG. 4: Characterization of Cy3-siRNA-targeted cell populations in mouse bone marrow.

FIGS. 5A-5B: Mouse bone marrow cells sorting: BM population after cells sorting based on their FSC/SSC parameters (5A). Cy3 siRNA in R1 and R2 populations after cell sorting (5B).

FIG. 6: Identification of siRNA positive mouse BM cells by their surface markers. R1 population represents siRNA positive BM cells while R2 population represents the bone marrow cells that were not detected by siRNA.

FIGS. 7A-7B: Phenotype of mouse bone marrow cells by May-Grünwald-Giemsa staining: phenotype of whole mouse BM cells (7A). Phenotype of the R1 population following cell sorting (7B).

FIGS. 8A-8C: Gr1+/CD11b+ cells expansion in the BM (8A) PB (8B) and spleen (8C).

FIGS. 9A-9B: Cy3 siRNA detection in BM cells of tumor-bearing mice. 9A. Cy3-siRNA detection in BM cells. 9B: Distribution of siRNA-positive cells in the BM based on Gr1 and CD11b expression (representative results of one out of three mice).

FIGS. 10A-10B: Cy3 siRNA detection in the spleen of tumor-bearing mice. 10A. Cy3-siRNA detection in the spleen. 10B. Distribution of siRNA-positive cells in the spleen based on Gr1 and CD11b expression (representative results of one out of three mice).

FIG. 11: Cy3 siRNA detection in the peripheral blood of tumor-bearing mice.

FIG. 12: Distribution of siRNA-positive cells in a tumor.

FIG. 13: Cy3-siRNA delivery to human MonoMac1 cells engrafted to NOD/SCID BM mice.

FIGS. 14A-14C: Cy3 siRNA delivery to human BM, PB and spleen cells of NOD/SCID chimeric mice.

FIGS. 15A-15B: CD11b+/Gr1+ cell expansion in the BM and the spleen of NOD/SCID/2 null mouse-bearing HCT116 tumor cells.

FIGS. 16A-16B: Cy3 siRNA in CD11b+ cells from tumor tissue. Purity of CD11b+Gr1+ cells after purification by CD11b microbeads (16A); siRNA positive CD11b cells from tumor tissue as observed by confocal microscopy. (16B).

### DETAILED DESCRIPTION OF THE INVENTION

The present invention provides a method for the treatment of various diseases and disorders associated with gene expression in a subset of bone marrow cells, and in particular for the treatment of tumors and leukemias and the prevention of allograft rejection. The present invention is based in part on the unexpected discovery that naked siRNA molecules target the bone marrow, and in particular a subset of bone marrow cells, when administered systemically. The discovery is surprising in view of the known obstacles to siRNA delivery.

For siRNA molecules to be effective in silencing mRNA of a target gene, the siRNA requires three levels of targeting: to the target tissue, to the target cell type and to the target subcellular compartment. The present invention now discloses systemic treatment of bone marrow diseases and disorders.

The present invention relates in general to compounds which down-regulate expression of genes expressed in immature myeloid cells, particularly to novel small interfering RNAs (siRNAs), and to the use of these novel siRNAs in the treatment of a subject suffering from medical conditions associated with expression of those genes in the immature myeloid cells.

Accordingly, in one aspect the present invention provides novel oligonucleotide sequences useful in inhibiting a gene selected from, whose mRNA polynucleotide sequences are set For each gene there is a table for 19-mer sequences, which are prioritized based on their score in the proprietary algorithm as the best sequences for targeting the human gene expression. 21- or 23-mer siRNA sequences can also be generated by 5′ and/or 3′ extension of the 19-mer sequences disclosed herein. Such extension is preferably complementary to the corresponding mRNA sequence. Certain 23-mer oligomers were devised by this method where the order of the prioritization is the order of the corresponding 19-mer.

Methods, molecules and compositions, which inhibit the genes of the invention, are discussed herein at length, and any of said molecules and/or compositions may be beneficially employed in the treatment of a subject suffering from any of said conditions.

The siRNAs of the present invention possess structures and modifications which may increase activity, increase stability, and or minimize toxicity; the novel modifications of the siRNAs of the present invention can be beneficially applied to double stranded RNA useful in preventing or attenuating target gene expression, in particular the target genes discussed herein.

Details of the target genes and their mRNA target is set forth in Tables A1 and A2, hereinbelow. The terms v1, v2 etc refers to splice variant mRNAs.

TABLE A1
Target genes for treatment of cancer and chronic inflammation

| Gene | Full name and Human Gene ID |
|---|---|
| MMP9 | matrix metallopeptidase 9 (gelatinase B, 92 kDa |
| | gelatinase, 92 kDa type IV collagenase |
| | gi\|74272286\|ref\|NM_004994.2 (SEQ ID NO: 1) |
| PROK2 | prokineticin 2, Bv8 |
| | gi\|187167258\|ref\|NM_021935.3 (SEQ ID NO: 2) |
| ARG1 | Arginase I |
| | gi\|10947138\|ref\|NM_000045.2 (SEQ ID NO: 3) |
| Nos-2 | nitric oxide synthase 2A |
| | gi\|206597519\|ref\|NM_000625.4 (SEQ ID NO: 4) |
| TGFβ1 | Transforming Growth Factor beta1 |
| | gi\|63025221\|ref\|NM_000660.3 (SEQ ID NO: 5) |
| STAT1 | signal transducer and activator of transcription 1 |
| | gi\|189458858\|ref\|NM_139266.2 (beta) (SEQ ID NO: 6) |
| | gi\|189458859\|ref\|NM_007315.3 (alpha) (SEQ ID NO: 7) |
| STAT3 | signal transducer and activator of transcription 3 |
| | gi\|47080104\|ref\|NM_139276.2\| (v 1) (SEQ ID NO: 8) |
| | gi\|47080105\|ref\|NM_003150.3 (v 2) (SEQ ID NO: 9) |
| STAT6 | signal transducer and activator of transcription 6 |
| | (IL4 induced) |
| | gi\|23397677\|ref\|NM_003153.3\| (SEQ ID NO: 10) |
| RAC1 | ras-related C3 botulinum toxin substrate 1 (rho family, |
| | small GTP binding protein) |
| | (gi\|38505164\|ref\|NM_198829.1) (SEQ ID NO: 11) |
| RAC2 | ras-related C3 botulinum toxin substrate 2 (rho family, |
| | small GTP binding protein Rac2) |
| | gi\|27881480\|ref\|NM_002872.3 (SEQ ID NO: 12) |
| NOX1 | NADPH oxidase 1 |
| | (gi:148536872, NM_007052.4 isoform 1L) |
| | (SEQ ID NO: 13) |
| | (gi:158536874, NM_013955.2 isoform 1Lv) |
| | (SEQ ID NO: 14) |
| NOX2 | NADPH oxidase 2 |
| (CYBB) | (gi:163854302, NM_000397.3) (SEQ ID NO: 15) |
| NOX3 | NADPH oxidase 3 |
| | gi\|11136625\|ref\|NM_015718.1 (SEQ ID NO: 16) |
| NOX4 | NADPH oxidase 4 |
| | (gi:20149638,NM_016931) (SEQ ID NO: 17) |
| NOX5 | NADPH oxidase 5 |
| | (gi:20127623, NM_024505) (SEQ ID NO: 18) |
| DUOX1 | dual oxidase 1 |
| | gi\|28872749\|ref\|NM_017434.3\| (SEQ ID NO: 19) |
| | gi\|28872750\|ref\|NM_175940.1\| (SEQ ID NO: 20) |
| DUOX2 | Dual oxidase 2 |
| | (gi:132566531, NM_014080) (SEQ ID NO: 21) |
| NOXO1 | NADPH oxidase organizer 1 |
| | (gi:34222190, variant a, NM_144603) (SEQ ID NO: 22) |
| | (gi:41281810, variant b, NM_172167) (SEQ ID NO: 23) |
| | (gi:41281827, variant c, NM_172168) (SEQ ID NO: 24) |
| NOXO2 | NADPH oxidase organizer 2 |
| (p47phox, | (gi:115298671, NM_000265) (SEQ ID NO: 25) |
| NCF1) | |
| NOXA1 | NADPH oxidase activator 1 |
| | (gi:41393186, NM_006647) (SEQ ID NO: 26) |
| NOXA2 | NADPH oxidase activator 2 (p67phox, NCF2) |
| | (gi:189083740, NM_000433.3) (SEQ ID NO: 27) |
| CYBA | cytochrome b-245, alpha polypeptide (p22phox) |
| | gi\|68509913\|ref\|NM_000101.2) (SEQ ID NO: 28) |
| ELA2 | neutrophil elastase |
| | gi\|58530849\|ref\|NM_001972.2\| (SEQ ID NO: 29) |
| Expi | extracellular proteinase inhibitor |
| | gi\|126366027\|ref\|NM_007969.4 (SEQ ID NO: 30) |
| LDLR | low density lipoprotein receptor (familial |
| | hypercholesterolemia) |
| | gi\|8051613\|ref\|NM_000527.2 (SEQ ID NO: 31) |
| TLR-1 | Toll-like receptor 1 |
| | gi\|41350336\|ref\|NM_003263.3 (SEQ ID NO: 32) |
| RLF | rearranged L-myc fusion |
| | gi\|157671948\|ref\|NM_012421.3 (SEQ ID NO: 33) |
| FGF13 | fibroblast growth factor 13 |
| | gi\|16306544\|ref\|NM_004114.2 (v 1A) (SEQ ID NO: 34) |
| | gi\|16306542\|ref\|NM_033642.1 (v 1B) (SEQ ID NO: 35) |
| IL-4R | interleukin 4 receptor |
| | gi\|56788410\|ref\|NM_001008699.1 (v 2) (SEQ ID NO: 36) |
| | gi\|56788409\|ref\|NM_000418.2 (v 1) (SEQ ID NO: 37) |
| IL-11R | interleukin 11 receptor, alpha |
| | gi\|22212920\|ref\|NM_004512.3 (v 1) (SEQ ID NO: 38) |
| | gi\|22212921\|ref\|NM_147162.1 (v 2) (SEQ ID NO: 39) |
| IL-13R | interleukin 13 receptor, alpha2 |
| | gi\|26787976\|ref\|NM_000640.2 (SEQ ID NO: 40) |
| IL-1R2 | interleukin 1 receptor, type II |
| | gi\|27894332\|ref\|NM_004633.3 (v 1) (SEQ ID NO: 41) |
| | gi\|27894333\|ref\|NM_173343.1 (v 2) (SEQ ID NO: 42) |
| IL-10 | interleukin 10 |
| | gi\|24430216\|ref\|NM_000572.2 (SEQ ID NO: 43) |
| IFN | Interferon alpha1 |
| | gi\|13128949\|ref\|NM_024013.1 (SEQ ID NO: 44) |
| TNFRSF18 | tumor necrosis factor receptor superfamily, member 18 |
| | gi\|23238190\|ref\|NM_004195.2 (v 1) (SEQ ID NO: 45) |
| | gi\|23238196\|ref\|NM_148902.1 (v 3) (SEQ ID NO: 46) |
| WNT5A | wingless-type MMTV integration site family, member 5A |
| | gi\|40806204\|ref\|NM_003392.3 (SEQ ID NO: 47) |
| SCAMP1 | Secretory carrier membrane 1 |
| | gi\|116256357\|ref\|NM_004866.4\| (SEQ ID NO: 48) |
| HSP86 | heat shock protein 90 kDa alpha (cytosolic), class A |
| | member 1 |
| | gi\|153792589\|ref\|NM_001017963.2 (v 1) (SEQ ID NO: 49) |
| | gi\|154146190\|ref\|NM_005348.3 (v 2) (SEQ ID NO: 50) |
| EGFR | Epidermal growth factor receptor (erythroblastic |
| | leukemia viral (v-erb-b) oncogene homolog, avian) |
| | gi\|41327737\|ref\|NM_005228.3 (v 1) (SEQ ID NO: 51) |
| | gi\|41327735\|ref\|NM_201284.1 (v 4) (SEQ ID NO: 52) |
| EphA5 | Ephrin receptor A5 |
| | gi\|56119208\|ref\|NM_004439.4 (v 1) (SEQ ID NO: 53) |
| | gi\|32967318\|ref\|NM_182472.1 (v 2) (SEQ ID NO: 54) |
| EphRB2 | Ephrin receptor B2 |
| | gi\|111118977\|ref\|NM_017449.3 (v 1) (SEQ ID NO: 55) |
| | gi\|111118979\|ref\|NM_004442.6 (v 2) (SEQ ID NO: 56) |
| Eph-RA7 | Eprhin receptor A7 |
| | gi\|205277372\|ref\|NM_004440.3 (SEQ ID NO: 57) |
| HGF | Hepatocyte growth factor (hepapoietin A; scatter factor) |
| | gi\|58533164\|ref\|NM_001010933.1 (v 4) (SEQ ID NO: 58) |
| | gi\|58533162\|ref\|NM_001010931.1 (v 2) (SEQ ID NO: 59) |
| ANGPTL6 | Angiopoietin Like-6 |
| | gi\|29893554\|ref\|NM_031917.2\| (SEQ ID NO: 60) |
| NTF5 | Neurotrophin 5 |
| | gi\|169658373\|ref\|NM_006179.4\| (SEQ ID NO: 61) |
| CLDN18 | Claudin-18 |
| | gi\|60115826\|ref\|NM_016369.3 (v 1) (SEQ ID NO: 62) |
| | gi\|60115825\|ref\|NM_001002026.2 (v 2) (SEQ ID NO: 63) |
| MDC15 | ADAM metallopeptidase domain 15 (metargidin) |
| | gi\|46909597\|ref\|NM_207196.1 (v 5) (SEQ ID NO: 64) |
| | gi\|46909599\|ref\|NM_207197.1 (v 6) (SEQ ID NO: 65) |
| MMRN1 | Multimerin 1 (ECM) |
| | gi\|45269140\|ref\|NM_007351.2 (SEQ ID NO: 66) |
| CD11b | integrin, alpha M (complement component 3 |
| | receptor 3 subunit) ITGAM |
| | gi\|88501733\|ref\|NM_000632.3 (SEQ ID NO: 67) |
| CD14 | CD 14 molecule |
| | gi\|91105163\|ref\|NM_000591.2\| (v 1) (SEQ ID NO: 68) |
| | gi\|91105158\|ref\|NM_001040021.1 (v 2) (SEQ ID NO: 69) |
| CD18 | CD18 leukocyte adhesion molecule |
| | gi\|47522671\|ref\|NM_213908.1 (SEQ ID NO: 70) |
| CD29 | integrin, beta 1 (fibronectin receptor, beta polypeptide, |
| (ITGB1) | antigen CD29 includes MDF2, MSK12) |
| | gi\|182519231\|ref\|NM_033666.1 (v 1B) (SEQ ID NO: 71) |
| | gi\|182519232\|ref\|NM_033667.1 (v 1C) (SEQ ID NO: 72) |
| CD120a | tumor necrosis factor receptor superfamily, member 1A |
| | gi\|23312372\|ref\|NM_001065.2 (SEQ ID NO: 73) |
| CD120b | tumor necrosis factor receptor superfamily, member 1B |
| | gi\|23312365\|ref\|NM_001066.2\| (SEQ ID NO: 74) |
| (Ep-1) | prostaglandin E receptor 1 (subtype EP1), 42 kDa |
| PTGER1 | gi\|38505193\|ref\|NM_000955.2 (SEQ ID NO: 75) |
| PEX-5 | peroxisomal biogenesis factor 5 |
| | gi\|196259768\|ref\|NM_000319.3 (SEQ ID NO: 76) |
| CD33 | CD33 molecule |
| | gi\|130979980\|ref\|NM_001772.3 (v 1) (SEQ ID NO: 77) |
| REG3A | Regenerating islet-derived-3a |
| | gi\|4505604\|ref\|NM_002580.1 (v 1) (SEQ ID NO: 78) |
| | gi\|21070992\|ref\|NM_138937.1 (v 3) (SEQ ID NO: 79) |
| PGK1 | phosphoglycerate kinase 1 |
| | gi\|183603937\|ref\|NM_000291.3\| (SEQ ID NO: 80) |
| ILRN1 | interleukin 1 receptor antagonist |
| | gi\|27894315\|ref\|NM_000577.3 (v 3) (SEQ ID NO: 81) |
| | gi\|27894316\|ref\|NM_173841.1 (v 2) (SEQ ID NO: 82) |
| HIF1a | ′hypoxia inducible factor 1, alpha subunit |
| | gi\|194473733\|ref\|NM_001530.3 (v 1) (SEQ ID NO: 83) |
| | gi\|194473734\|ref\|NM_181054.2 (v 2) (SEQ ID NO: 84) |
| CD80 | CD80 molecule |
| | gi\|113722122\|ref\|NM_005191.3 (SEQ ID NO: 87) |
| CASP2 | caspase 2, apoptosis-related cysteine peptidase |
| | gi\|39995058\|ref\|NM_032982.2 (SEQ ID NO: 85) |
| | gi\|39995060\|ref\|NM_032983.2 (SEQ ID NO: 86) |

TABLE A2
Target genes for treatment of allograft rejection

| Gene | Full name and Human Gene ID |
|---|---|
| CD80 | CD80 molecule |
| | gi\|113722122\|ref\|NM_005191.3 (SEQ ID NO: 87) |
| CD86 | CD86 molecule |
| | gi\|91208429\|ref\|NM_175862.3 (v 1) (SEQ ID NO: 88) |
| | gi\|91208432\|ref\|NM_006889.3 (v 2) (SEQ ID NO: 89) |

Tables A1 and A2 provide the gi (GeneInfo identifier) and accession numbers for polynucleotide sequences of human mRNA to which the oligonucleotide inhibitors of the present invention are directed. (“v” refers to transcript variant)

Inhibition of the genes in Tables A1 and A2 is useful in treating cancer and transplant rejection, respectively.

### DEFINITIONS

For convenience certain terms employed in the specification, examples and claims are described herein.

It is to be noted that, as used herein, the singular forms “a”, “an” and “the” include plural forms unless the content clearly dictates otherwise.

Where aspects or embodiments of the invention are described in terms of Markush groups or other grouping of alternatives, those skilled in the art will recognize that the invention is also thereby described in terms of any individual member or subgroup of members of the group.

An “inhibitor” is a compound which is capable of reducing the expression of a gene or the activity of the product of such gene to an extent sufficient to achieve a desired biological or physiological effect. The term “inhibitor” as used herein refers to one or more of an oligonucleotide inhibitor, including siRNA, shRNA, miRNA and ribozymes. Inhibition may also be referred to as down-regulation or, for RNAi, silencing.

The term “inhibit” as used herein refers to reducing the expression of a gene, a variant thereof or the activity of the product of such gene to an extent sufficient to achieve a desired biological or physiological effect. Inhibition may be complete or partial. For example “inhibition” of a NOX gene means inhibition of the gene expression (transcription or translation) or polypeptide activity of a gene selected from the group NOX4, NOX1, NOX2 (gp91phox, CYBB), NOX3, NOX5, DUOX2, NOXO1, NOXO2, NOXA1 and NOXA2 (p67phox), or SNP (single nucleotide polymorphism) or other variants thereof.

As used herein, the terms “polynucleotide” and “nucleic acid” may be used interchangeably and refer to nucleotide sequences comprising deoxyribonucleic acid (DNA), and ribonucleic acid (RNA). The terms should also be understood to include, as equivalents, analogs of either RNA or DNA made from nucleotide analogs. Throughout this application mRNA sequences are set forth as representing the corresponding genes. The terms “mRNA polynucleotide sequence” and mRNA are used interchangeably.

“Oligonucleotide” or “oligomer” refers to a deoxyribonucleotide or ribonucleotide sequence from about 2 to about 50 nucleotides. Each DNA or RNA nucleotide may be independently natural or synthetic, and or modified or unmodified. Modifications include changes to the sugar moiety, the base moiety and or the linkages between nucleotides in the oligonucleotide. The compounds of the present invention encompass molecules comprising deoxyribonucleotides, ribonucleotides, modified deoxyribonucleotides, modified ribonucleotides and combinations thereof.

The present invention provides methods and compositions for inhibiting expression of a target gene in vivo. In general, the method includes administering oligoribonucleotides, in particular small interfering RNAs (i.e., siRNAs) or a nucleic acid material that can produce siRNA in a cell, to target an mRNA set forth in Tables A1 and A2; in an amount sufficient to down-regulate expression of a target gene by an RNA interference mechanism. In particular, the method can be used to inhibit expression of the gene for treatment of a subject suffering from a disease related to expression of that gene. In accordance with the present invention, the siRNA molecules or inhibitors of the target gene are used as drugs to treat various pathologies.

“Nucleotide” is meant to encompass deoxyribonucleotides and ribonucleotides, which may be natural or synthetic, and or modified or unmodified. Modifications include changes and substitutions to the sugar moiety, the base moiety and/or the internucleotide linkages.

All analogs of, or modifications to, a nucleotide/oligonucleotide may be employed with the present invention, provided that said analog or modification does not substantially adversely affect the function of the nucleotide/oligonucleotide. Acceptable modifications include modifications of the sugar moiety, modifications of the base moiety, modifications in the internucleotide linkages and combinations thereof.

According to one aspect the present invention provides inhibitory oligonucleotide compounds comprising unmodified and modified nucleotides. The compound comprises at least one modified nucleotide selected from the group consisting of a sugar modification, a base modification and an internucleotide linkage modification and may contain DNA, and modified nucleotides such as LNA (locked nucleic acid) including ENA (ethylene-bridged nucleic acid; PNA (peptide nucleic acid); arabinoside; PACE (phosphonoacetate and derivatives thereof), mirror nucleotide, or nucleotides with a 6 carbon sugar.

In one embodiment the compound comprises a 2′ modification on the sugar moiety of at least one ribonucleotide (“2′ sugar modification”). In certain embodiments the compound comprises 2′O-alkyl or 2′-fluoro or 2′O-allyl or any other 2′ sugar modification, optionally on alternate positions.

Other stabilizing modifications are also possible (eg. modified nucleotides added to a 3′ or 5′ terminus of an oligomer). In some embodiments the backbone of the oligonucleotides is modified and comprises phosphate-D-ribose entities but may also contain thiophosphate-D-ribose entities, triester, thioate, 2′-5′ bridged backbone (also may be referred to as 5′-2′), PACE modified internucleotide linkage or any other type of modification.

Other modifications include additions to the 5′ and/or 3′ termini of the oligonucleotides. Such terminal modifications may be lipids, peptides, sugars or other molecules.

The present invention also relates to compounds which down-regulate expression of various genes, particularly to novel small interfering RNAs (siRNAs), and to the use of these novel siRNAs in the treatment of cancer and transplant rejection.

Lists of preferred siRNA to be used in the present invention are provided in Tables B ( ) For each gene there is a list of 19-mer sequences (for CD80 and CD86 there are also 21 and 23 mer sequences), which are prioritized based on their score in the proprietary algorithm as the best sequences for targeting the human gene expression. A 21- or 23-mer siRNA sequence can also be generated by 5′ and/or 3′ extension of the 19-mer sequences disclosed herein. Such extension is preferably complementary to the corresponding mRNA sequence. Certain 23-mer oligomers were devised by this method where the order of the prioritization is the order of the corresponding 19-mer. A full list of 21 and 23-mer sequences was provided in the U.S. Ser. No. 61/116,806, which is hereby incorporated by reference in its entirety.

### Cancer

The present invention relates to the treatment of cancer in a subject which comprises administering systemically to the subject a therapeutically effective amount of an oligonucleotide which inhibits expression of a gene expressed in a myeloid cell in the subject in an amount effective to treat the cancer. The terms “cancer” and “cancerous” refer to or describe the physiological condition in mammals that is typically characterized by unregulated cell growth. Examples of cancer include but are not limited to, carcinoma, lymphoma, blastoma, sarcoma, and leukemia or lymphoid malignancies. Other examples of such cancers include kidney or renal cancer, breast cancer, colon cancer, rectal cancer, colorectal cancer, lung cancer including small-cell lung cancer, non-small cell lung cancer, adenocarcinoma of the lung and squamous carcinoma of the lung, squamous cell cancer (e.g. epithelial squamous cell cancer), cervical cancer, ovarian cancer, prostate cancer, liver cancer, bladder cancer, cancer of the peritoneum, hepatocellular cancer, gastric or stomach cancer including gastrointestinal cancer, gastrointestinal stromal tumors (GIST), pancreatic cancer, head and neck cancer, glioblastoma, retinoblastoma, astrocytoma, thecomas, arrhenoblastomas, hepatoma, hematologic malignancies including non-Hodgkins lymphoma (NHL), multiple myeloma and acute hematologic malignancies, endometrial or uterine carcinoma, endometriosis, fibrosarcomas, choriocarcinoma, salivary gland carcinoma, vulval cancer, thyroid cancer, esophageal carcinomas, hepatic carcinoma, anal carcinoma, penile carcinoma, nasopharyngeal carcinoma, laryngeal carcinomas, Kaposi's sarcoma, melanoma, skin carcinomas, Schwannoma, oligodendroglioma, neuroblastomas, rhabdomyosarcoma, osteogenic sarcoma, leiomyosarcomas, urinary tract carcinomas, thyroid carcinomas, Wilm's tumor, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia); chronic lymphocytic leukemia (CLL); acute lymphoblastic leukemia (ALL); Hairy cell leukemia; chronic myeloblastic leukemia; and post-transplant lymphoproliferative disorder (PTLD), as well as abnormal vascular proliferation associated with phakomatoses, edema (such as that associated with brain tumors), and Meigs' syndrome. “Tumor”, as used herein, refers to all neoplastic cell growth and proliferation, whether malignant or benign, and all pre-cancerous and cancerous cells and tissues.

### Oligonucleotides

Tables B and G comprise nucleic acid sequences of sense and corresponding antisense oligomers, useful in preparing siRNA compounds. The compounds are used per se, as chemically and or structurally modified compounds.

The selection and synthesis of siRNA corresponding to known genes has been widely reported; see for example Ui-Tei et al., J Biomed Biotechnol. 2006; 65052; Chalk et al., BBRC. 2004, 319(1):264-74; Sioud & Leirdal, Met. Mol. Biol. 2004, 252:457-69; Levenkova et al., Bioinform. 2004, 20(3):430-2; Ui-Tei et al., NAR. 2004, 32(3):936-48. For examples of the use and production of modified siRNA see for example Braasch et al., Biochem. 2003, 42(26):7967-75; Chiu et al., RNA. 2003, 9(9):1034-48; PCT Publication Nos. WO 2004/015107 and WO 02/44321 and U.S. Pat. Nos. 5,898,031 and 6,107,094.

The present invention provides double-stranded oligonucleotides (e.g. siRNAs), which down-regulate the expression of a target gene. An siRNA of the invention is a duplex oligoribonucleotide in which the sense strand is derived from the mRNA sequence of the target gene, and the antisense strand is complementary to the sense strand. In general, some deviation from the target mRNA sequence is tolerated without compromising the siRNA activity (see e.g. Czauderna et al., NAR. 2003, 31(11):2705-2716). Without wishing to be bound to theory, an siRNA of the invention inhibits gene expression on a post-transcriptional level with or without destroying the mRNA and an siRNA may target the mRNA for specific cleavage and degradation and/or may inhibit translation from the targeted message.

In some embodiments an oligonucleotide pair selected from Tables B or G (set forth in SEQ ID NOS:90-24,117) comprises modified siRNA, having one or more of any of the modifications disclosed herein. In various embodiments the siRNA comprises an RNA duplex comprising a first strand and a second strand, whereby the first strand comprises a ribonucleotide sequence at least partially complementary to about 18 to about 40 consecutive nucleotides of a target nucleic acid which is mRNA transcribed from a target gene, and the second strand comprises a ribonucleotide sequence at least partially complementary to the first strand and wherein said first strand and or said second strand comprises a plurality of groups of modified ribonucleotides, optionally having a modification at the 2′-position of the sugar moiety whereby within each strand each group of modified ribonucleotides is flanked on one or both sides by a group of flanking nucleotides, optionally ribonucleotides, whereby each ribonucleotide forming the group of flanking ribonucleotides is selected from an unmodified ribonucleotide or a ribonucleotide having a modification different from the modification of the groups of modified ribonucleotides.

The group of modified ribonucleotides and/or the group of flanking nucleotides may comprise a number of ribonucleotides selected from the group consisting of an integer from 1 to 12. Accordingly, the group thus comprises one nucleotide, two nucleotides, three nucleotides, four nucleotides, five nucleotides, six nucleotides, seven nucleotides, eight nucleotides, nine nucleotides, ten nucleotides, eleven nucleotides or twelve nucleotides.

The groups of modified nucleotides and flanking nucleotides may be organized in a pattern on one or both of the strands. In some embodiments the antisense and sense strands comprise alternating unmodified and 2′ sugar modified ribonucleotides. In some preferred embodiments the middle ribonucleotide in the antisense strand is an unmodified nucleotide. For example, in a 19-oligomer antisense strand, ribonucleotide at position 10 is unmodified; in a 21-oligomer antisense strand, the ribonucleotide at position 11 is unmodified; and in a 23-oligomer antisense strand, ribonucleotide at position 12 is unmodified. The modifications or pattern of modification, if any, of the siRNA must be planned to allow for this. In an even-numbered oligomer, e.g. a 22 mer, the middle nucleotide may be at position 11 or 12.

Possible modifications on the 2′ moiety of the sugar residue include amino, fluoro, methoxy alkoxy, alkyl, amino, fluoro, chloro, bromo, CN, CF, imidazole, carboxylate, thioate, C₁ to C₁₀ lower alkyl, substituted lower alkyl, alkaryl or aralkyl, OCF₃, OCN, O-, S-, or N-alkyl; O-, S, or N-alkenyl; SOCH₃; SO₂CH₃; ONO₂; NO₂, N₃; heterozycloalkyl; heterozycloalkaryl; aminoalkylamino; polyalkylamino or substituted silyl, as, among others, described in European patents EP 0 586 520 B1 or EP 0 618 925 B1. One or more deoxynucleotides are also tolerated in the compounds of the present invention. As used herein, in the description of any strategy for the design of molecules, RNAi or any embodiment of RNAi disclosed herein, the term “end modification” means a chemical entity added to the terminal 5′ or 3′ nucleotide of the sense and/or antisense strand. Examples for such end modifications include, but are not limited to, 3′ or 5′ phosphate, inverted abasic, abasic, amino, fluoro, chloro, bromo, CN, CF₃, methoxy, imidazolyl, carboxylate, phosphothioate, C₁ to C₂₂ and lower alkyl, lipids, sugars and polyaminoacids (i.e. peptides), substituted lower alkyl, alkaryl or aralkyl, OCF₃, OCN, O-, S-, or N-alkyl; O-, S-, or N-alkenyl; SOCH₃; SO₂CH₃; ONO₂; NO₂, N₃; heterocycloalkyl; heterocycloalkaryl; aminoalkylamino; polyalkylamino or substituted silyl, as, among others, described in European patents EP 0 586 520 B1 or EP 0 618 925 B1.

In some embodiments the siRNA is blunt ended, i.e. Z and Z′ are absent, on one or both ends. More specifically, the siRNA may be blunt ended on the end defined by the 5′-terminus of the first strand and the 3′-terminus of the second strand, and/or the end defined by the 3′-terminus of the first strand and the 5′-terminus of the second strand.

In other embodiments at least one of the two strands may have an overhang of at least one nucleotide at the 5′-terminus; the overhang may consist of at least one deoxyribonucleotide. At least one of the strands may also optionally have an overhang of at least one nucleotide at the 3′-terminus. The overhang may consist of from about 1 to about 5 nucleotides.

The length of siRNA duplex is from about 18 to about 40 ribonucleotides, preferably 19, 21 or 23 ribonucleotides. Further, the length of each strand may independently have a length selected from the group consisting of about 15 to about 40 bases, preferably 18 to 23 bases and more preferably 19 bases (modified or unmodified or a combination).

In certain embodiments the complementarity between said first strand and the target nucleic acid is perfect. In some embodiments, the strands are substantially complementary, i.e. having one, two or up to three mismatches between said first strand and the target nucleic acid. Substantially complementary refers to complementarity of greater than about 84%, to another sequence. For example in a duplex region consisting of 19 base pairs one mismatch results in 94.7% complementarity, two mismatches results in about 89.5% complementarity and 3 mismatches results in about 84.2% complementarity, rendering the duplex region substantially complementary. Accordingly substantially identical refers to identity of greater than about 84%, to another sequence. Thus, the invention provides siRNA comprising a nucleic acid sequence set forth in Tables B and G; wherein 1, 2, or 3 of the nucleotides in one strand or both strands are substituted thereby providing at least one base pair mismatch. The substituted nucleotides in each strand are preferably in the terminal region of one strand or both strands.

The first strand and the second strand may be linked by a loop structure, which may be comprised of a non-nucleic acid polymer such as, inter alia, polyethylene glycol. Alternatively, the loop structure may be comprised of a nucleic acid, including modified and non-modified ribonucleotides and modified and non-modified deoxyribonucleotides.

Further, the 5′-terminus of the first strand of the siRNA may be linked to the 3′-terminus of the second strand, or the 3′-terminus of the first strand may be linked to the 5′-terminus of the second strand, said linkage being via a nucleic acid linker typically having a length between 2-100 nucleobases, preferably about 2 to about 30 nucleobases.

In preferred embodiments of the compounds of the invention having alternating ribonucleotides modified in at least one of the antisense and the sense strands of the compound, for 19 mer and 23 mer oligomers the ribonucleotides at the 5′ and 3′ termini of the antisense strand are modified in their sugar residues, and the ribonucleotides at the 5′ and 3′ termini of the sense strand are unmodified in their sugar residues. For 21 mer oligomers the ribonucleotides at the 5′ and 3′ termini of the sense strand are modified in their sugar residues, and the ribonucleotides at the 5′ and 3′ termini of the antisense strand are unmodified in their sugar residues, or may have an optional additional modification at the 3′ terminus. As mentioned above, it is preferred that the middle nucleotide of the antisense strand is unmodified.

According to one preferred embodiment of the invention, the antisense and the sense strands of the oligonucleotide/siRNA are phosphorylated only at the 3′-terminus and not at the 5′-terminus. According to another preferred embodiment of the invention, the antisense and the sense strands are non-phosphorylated. According to yet another preferred embodiment of the invention, the 5′ most ribonucleotide in the sense strand is modified to abolish any possibility of in vivo 5′-phosphorylation.

Any siRNA sequence disclosed herein can be prepared having any of the modifications/structures disclosed herein. The combination of sequence plus structure is novel and can be used in the treatment of the conditions disclosed herein.

siRNA Structures

The selection and synthesis of siRNA corresponding to known genes has been widely reported; (see for example Ui-Tei et al., J Biomed Biotech. 2006; 2006: 65052; Chalk et al., BBRC. 2004, 319(1): 264-74; Sioud & Leirdal, Met. Mol. Biol.; 2004, 252:457-69; Levenkova et al., Bioinform. 2004, 20(3):430-2; Ui-Tei et al., NAR. 2004, 32(3):936-48).

For examples of the use of, and production of, modified siRNA see for example Braasch et al., Biochem. 2003, 42(26):7967-75; Chiu et al., RNA, 2003, 9(9):1034-48; PCT publications WO 2004/015107 (atugen AG) and WO 02/44321 (Tuschl et al). U.S. Pat. Nos. 5,898,031 and 6,107,094, teach chemically modified oligomers. US Patent Publication Nos. 2005/0080246 and 2005/0042647 relate to oligomeric compounds having an alternating motif and dsRNA compounds having chemically modified internucleoside linkages, respectively.

Other modifications have been disclosed. The inclusion of a 5′-phosphate moiety was shown to enhance activity of siRNAs in Drosophila embryos (Boutla, et al., Curr. Biol. 2001, 11:1776-1780) and is required for siRNA function in human HeLa cells (Schwarz et al., Mol. Cell, 2002, 10:537-48). Amarzguioui et al., (NAR, 2003, 31(2):589-95) showed that siRNA activity depended on the positioning of the 2′-O-methyl modifications. Holen et al (NAR. 2003, 31(9):2401-07) report that an siRNA having small numbers of 2′-O-methyl modified nucleosides gave good activity compared to wild type but that the activity decreased as the numbers of 2′-O-methyl modified nucleosides was increased. Chiu and Rana (RNA. 2003, 9:1034-48) teach that incorporation of 2′-O-methyl modified nucleosides in the sense or antisense strand (fully modified strands) severely reduced siRNA activity relative to unmodified siRNA. The placement of a 2′-O-methyl group at the 5′-terminus on the antisense strand was reported to severely limit activity whereas placement at the 3′-terminus of the antisense and at both termini of the sense strand was tolerated (Czaudema et al., NAR. 2003, 31(11):2705-16). The molecules of the present invention offer an advantage in that they are active and or stable, are non-toxic and may be formulated as pharmaceutical compositions for treatment of various diseases.

The nucleotides can be selected from naturally occurring or synthetic modified bases. Naturally occurring bases include adenine, guanine, cytosine, thymine and uracil. Modified bases of nucleotides include inosine, xanthine, hypoxanthine, 2-aminoadenine, 6-methyl, 2-propyl and other alkyl adenines, 5-halo uracil, 5-halo cytosine, 6-aza cytosine and 6-aza thymine, pseudo uracil, 4-thiouracil, 8-halo adenine, 8-aminoadenine, 8-thiol adenine, 8-thiolalkyl adenines, 8-hydroxyl adenine and other 8-substituted adenines, 8-halo guanines, 8-amino guanine, 8-thiol guanine, 8-thioalkyl guanines, 8-hydroxyl guanine and other substituted guanines, other aza and deaza adenines, other aza and deaza guanines, 5-trifluoromethyl uracil and 5-trifluoro cytosine.

In addition, analogues of polynucleotides can be prepared wherein the structure of one or more nucleotide is fundamentally altered and better suited as therapeutic or experimental reagents. An example of a nucleotide analog is a peptide nucleic acid (PNA) wherein the deoxyribose (or ribose) phosphate backbone in DNA (or RNA is replaced with a polyamide backbone which is similar to that found in peptides. PNA analogs have been shown to be resistant to enzymatic degradation and to have extended lives in vivo and in vitro.

Possible modifications to the sugar residue are manifold and include 2′-O alkyl, locked nucleic acid (LNA), glycol nucleic acid (GNA), threose nucleic acid (TNA), arabinoside, altritol (ANA) and other, 6-membered sugars including morpholinos, and cyclohexinyls.

LNA compounds are disclosed in International Patent Publication Nos. WO 00/47599, WO 99/14226, and WO 98/39352. Examples of siRNA compounds comprising LNA nucleotides are disclosed in Elmen et al., (NAR 2005. 33(1):439-447) and in PCT Patent Publication No. WO 2004/083430.

The compounds of the present invention can be synthesized using one or more inverted nucleotides, for example inverted thymidine or inverted adenine (for example see Takei, et al., 2002. JBC 277(26):23800-06.

In the context of the present invention, a “mirror” nucleotide also referred to as a spiegelmer, is a nucleotide with reverse chirality to the naturally occurring or commonly employed nucleotide, i.e., a mirror image of the naturally occurring or commonly employed nucleotide. The mirror nucleotide can be a ribonucleotide (L-RNA) or a deoxyribonucleotide (L-DNA) and may further comprise at least one sugar, base and or backbone modification. U.S. Pat. No. 6,602,858 discloses nucleic acid catalysts comprising at least one L-nucleotide substitution.

Backbone modifications, such as ethyl (resulting in a phospho-ethyl triester); propyl (resulting in a phospho-propyl triester); and butyl (resulting in a phospho-butyl triester) are also possible. Other backbone modifications include polymer backbones, cyclic backbones, acyclic backbones, thiophosphate-D-ribose backbones, amidates, phosphonoacetate derivatives. Certain structures include siRNA compounds having one or a plurality of 2′-5′ internucleotide linkages (bridges or backbone).

Additional modifications which may be present in the molecules of the present invention include nucleoside modifications such as artificial nucleic acids, peptide nucleic acid (PNA), morpholino and locked nucleic acid (LNA), glycol nucleic acid (GNA), threose nucleic acid (TNA), arabinoside, and mirror nucleoside (for example, beta-L-deoxynucleoside instead of beta-D-deoxynucleoside Further, said molecules may additionally contain modifications on the sugar, such as 2′ alkyl, 2′ fluoro, 2′O allyl, 2′ amine and 2′ alkoxy. Additional sugar modifications are discussed herein.

Further, the inhibitory nucleic acid molecules of the present invention may comprise one or more gaps and/or one or more nicks and/or one or more mismatches. Without wishing to be bound by theory, gaps, nicks and mismatches have the advantage of partially destabilizing the nucleic acid/siRNA, so that it may be more easily processed by endogenous cellular machinery such as DICER, DROSHA or RISC into its inhibitory components.

The molecules of the present invention may comprise siRNAs, synthetic siRNAs, shRNAs and synthetic shRNAs, in addition to other nucleic acid sequences or molecules which encode such molecules or other inhibitory nucleotide molecules.

The compounds of the present invention may further comprise an end modification. A biotin group may be attached to either the most 5′ or the most 3′ nucleotide of the first and/or second strand or to both ends. In a more preferred embodiment the biotin group is coupled to a polypeptide or a protein. It is also within the scope of the present invention that the polypeptide or protein is attached through any of the other aforementioned modifications.

The various end modifications as disclosed herein are preferably located at the ribose moiety of a nucleotide of the nucleic acid according to the present invention. More particularly, the end modification may be attached to or replace any of the OH-groups of the ribose moiety, including but not limited to the 2′OH, 3′OH and 5′OH position, provided that the nucleotide thus modified is a terminal nucleotide. Inverted abasic or abasic are nucleotides, either deoxyribonucleotides or ribonucleotides which do not have a nucleobase moiety. This kind of compound is, inter alia, described in Sternberger, et al., (Antisense Nucleic Acid Drug Dev, 2002.12, 131-43).

In the context of the present invention, a gap in a nucleic acid refers to the absence of one or more internal nucleotides in one strand, while a nick in a nucleic acid refers to the absence of an internucleotide linkage between two adjacent nucleotides in one strand. Any of the molecules of the present invention may contain one or more gaps and/or one or more nicks. Further provided by the present invention is an siRNA encoded by any of the molecules disclosed herein, a vector encoding any of the molecules disclosed herein, and a pharmaceutical composition comprising any of the molecules disclosed herein or the vectors encoding them; and a pharmaceutically acceptable carrier.

Particular molecules to be administered according to the methods of the present invention are disclosed below under the heading “structural motifs”. For the sake of clarity, any of these molecules can be administered according to any of the methods of the present invention.

### Structural Motifs

According to the present invention the siRNA compounds that are chemically and or structurally modified according to one of the following modifications set forth in Structures (B)-(P) or as tandem siRNA or RNAstar (see below) are useful in the methods of the present invention.

In one aspect the present invention provides a compound set forth as Structure (A):

| | | | | |
|---|---|---|---|---|
| (A) | 5′ | (N)ₓ-Z | 3′ | (antisense strand) |
| | 3′ | Z′-(N′)_{y} | 5′ | (sense strand) |

wherein each of N and N′ is a nucleotide selected from an unmodified ribonucleotide, a modified ribonucleotide, an unmodified deoxyribonucleotide and a modified deoxyribonucleotide;
wherein each of (N)ₓ and (N′)_{y} is an oligonucleotide in which each consecutive N or N′ is joined to the next N or N′ by a covalent bond;
wherein each of x and y is an integer between 18 and 40;
wherein each of Z and Z′ may be present or absent, but if present is 1-5 consecutive nucleotides covalently attached at the 3′ terminus of the strand in which it is present; and
wherein the sequence of (N)ₓ comprises an antisense sequence substantially complementary to about 18 to about 40 consecutive ribonucleotides in the mRNA transcribed from a gene.

In certain embodiments the present invention provides a compound having structure B:

| | | | | |
|---|---|---|---|---|
| (B) | 5′ | (N)x-Z | 3′ | antisense strand |
| | 3′ | Z′-(N′)y | 5′ | sense strand |

wherein each of (N)ₓ and (N′)_{y} is an oligomer in which each consecutive N or N′ is an unmodified ribonucleotide or a modified ribonucleotide joined to the next N or N′ by a covalent bond;
wherein each of x and y is an integer between 18 and 40;
wherein (N)ₓ and (N′)_{y} are fully complementary
wherein each of Z and Z′ may be present or absent, but if present is 1-5 consecutive nucleotides covalently attached at the 3′ terminus of the strand in which it is present;
wherein alternating ribonucleotides in each of (N)ₓ and (N′)_{y} are modified to result in a 2′-O-methyl modification in the sugar residue of the modified ribonucleotides;
wherein the sequence of (N′)_{y} is a sequence complementary to (N)x; and wherein the sequence of (N)ₓ comprises an antisense sequence substantially complementary to about 18 to about 40 consecutive ribonucleotides in the mRNA transcribed from a gene. In some embodiments each of (N)ₓ and (N′)_{y} is independently phosphorylated or non-phosphorylated at the 3′ and 5′ termini.

In certain embodiments of the invention, alternating ribonucleotides are modified in both the antisense and the sense strands of the compound.

In certain embodiments wherein each of x and y=19 or 23, each N at the 5′ and 3′ termini of (N)ₓ is modified; and

each N′ at the 5′ and 3′ termini of (N′)_{y} is unmodified.

In particular embodiments, when x and y=19, the siRNA is modified such that a 2′-O-methyl (2′-OMe) group is present on the first, third, fifth, seventh, ninth, eleventh, thirteenth, fifteenth, seventeenth and nineteenth nucleotide of the antisense strand (N)ₓ, and whereby the very same modification, i.e. a 2′-OMe group, is present at the second, fourth, sixth, eighth, tenth, twelfth, fourteenth, sixteenth and eighteenth nucleotide of the sense strand (N′)_{y}. In various embodiments these particular siRNA compounds are blunt ended at both termini.

In some embodiments, the present invention provides a compound having Structure (C):

| | | | | |
|---|---|---|---|---|
| (C) | 5′ | (N)x-Z | 3′ | antisense strand |
| | 3′ | Z′-(N′)y | 5′ | sense strand |

wherein each of N and N′ is a nucleotide independently selected from an unmodified ribonucleotide, a modified ribonucleotide, an unmodified deoxyribonucleotide and a modified deoxyribonucleotide;
wherein each of (N)x and (N′)y is an oligomer in which each consecutive nucleotide is joined to the next nucleotide by a covalent bond and each of x and y is an integer between 18 and 40;
wherein in (N)x the nucleotides are unmodified or (N)x comprises alternating modified ribonucleotides and unmodified ribonucleotides; each modified ribonucleotide being modified so as to have a 2′-O-methyl on its sugar and the ribonucleotide located at the middle position of (N)x being modified or unmodified preferably unmodified;
wherein (N′)y comprises unmodified ribonucleotides further comprising one modified nucleotide at a terminal or penultimate position, wherein the modified nucleotide is selected from the group consisting of a mirror nucleotide, a bicyclic nucleotide, a 2′-sugar modified nucleotide, an altritol nucleotide, or a nucleotide joined to an adjacent nucleotide by an internucleotide linkage selected from a 2′-5′ phosphodiester bond, a P-alkoxy linkage or a PACE linkage;
wherein if more than one nucleotide is modified in (N′)y, the modified nucleotides may be consecutive;
wherein each of Z and Z′ may be present or absent, but if present is 1-5 deoxyribonucleotides covalently attached at the 3′ terminus of any oligomer to which it is attached;
wherein the sequence of (N′)_{y} comprises a sequence substantially complementary to (N)x; and wherein the sequence of (N)ₓ comprises an antisense sequence substantially complementary to about 18 to about 40 consecutive ribonucleotides in the mRNA transcribed from a gene.

In particular embodiments, x=y=19 and in (N)x each modified ribonucleotide is modified so as to have a 2′-O-methyl on its sugar and the ribonucleotide located at the middle of (N)x is unmodified. Accordingly, in a compound wherein x=19, (N)x comprises 2′-O-methyl sugar modified ribonucleotides at positions 1, 3, 5, 7, 9, 11, 13, 15, 17 and 19. In other embodiments, (N)x comprises 2′O Me modified ribonucleotides at positions 2, 4, 6, 8, 11, 13, 15, 17 and 19 and may further comprise at least one abasic or inverted abasic pseudo-nucleotide for example in position 5. In other embodiments, (N)x comprises 2′O Me modified ribonucleotides at positions 2, 4, 8, 11, 13, 15, 17 and 19 and may further comprise at least one abasic or inverted abasic pseudo-nucleotide for example in position 6. In other embodiments, (N)x comprises 2′O Me modified ribonucleotides at positions 2, 4, 6, 8, 11, 13, 17 and 19 and may further comprise at least one abasic or inverted abasic pseudo-nucleotide for example in position 15. In other embodiments, (N)x comprises 2′O Me modified ribonucleotides at positions 2, 4, 6, 8, 11, 13, 15, 17 and 19 and may further comprise at least one abasic or inverted abasic pseudo-nucleotide for example in position 14. In other embodiments, (N)x comprises 2′O Me modified ribonucleotides at positions 1, 2, 3, 7, 9, 11, 13, 15, 17 and 19 and may further comprise at least one abasic or inverted abasic pseudo-nucleotide for example in position 5. In other embodiments, (N)x comprises 2′O Me modified ribonucleotides at positions 1, 2, 3, 5, 7, 9, 11, 13, 15, 17 and 19 and may further comprise at least one abasic or inverted abasic pseudo-nucleotide for example in position 6. In other embodiments, (N)x comprises 2′O Me modified ribonucleotides at positions 1, 2, 3, 5, 7, 9, 11, 13, 17 and 19 and may further comprise at least one abasic or inverted abasic pseudo-nucleotide for example in position 15. In other embodiments, (N)x comprises 2′O Me modified ribonucleotides at positions 1, 2, 3, 5, 7, 9, 11, 13, 15, 17 and 19 and may further comprise at least one abasic or inverted abasic pseudo-nucleotide for example in position 14. In other embodiments, (N)x comprises 2′O Me modified ribonucleotides at positions 2, 4, 6, 7, 9, 11, 13, 15, 17 and 19 and may further comprise at least one abasic or inverted abasic pseudo-nucleotide for example in position 5. In other embodiments, (N)x comprises 2′O Me modified ribonucleotides at positions 1, 2, 4, 6, 7, 9, 11, 13, 15, 17 and 19 and may further comprise at least one abasic or inverted abasic pseudo-nucleotide for example in position 5. In other embodiments, (N)x comprises 2′O Me modified ribonucleotides at positions 2, 4, 6, 8, 11, 13, 14, 16, 17 and 19 and may further comprise at least one abasic or inverted abasic pseudo-nucleotide for example in position 15. In other embodiments, (N)x comprises 2′O Me modified ribonucleotides at positions 1, 2, 3, 5, 7, 9, 11, 13, 14, 16, 17 and 19 and may further comprise at least one abasic or inverted abasic pseudo-nucleotide for example in position 15. In other embodiments, (N)x comprises 2′O Me modified ribonucleotides at positions 2, 4, 6, 8, 11, 13, 15, 17 and 19 and may further comprise at least one abasic or inverted abasic pseudo-nucleotide for example in position 7. In other embodiments, (N)x comprises 2′O Me modified ribonucleotides at positions 2, 4, 6, 11, 13, 15, 17 and 19 and may further comprise at least one abasic or inverted abasic pseudo-nucleotide for example in position 8. In other embodiments, (N)x comprises 2′O Me modified ribonucleotides at positions 2, 4, 6, 8, 11, 13, 15, 17 and 19 and may further comprise at least one abasic or inverted abasic pseudo-nucleotide for example in position 9. In other embodiments, (N)x comprises 2′O Me modified ribonucleotides at positions 2, 4, 6, 8, 11, 13, 15, 17 and 19 and may further comprise at least one abasic or inverted abasic pseudo-nucleotide for example in position 10. In other embodiments, (N)x comprises 2′O Me modified ribonucleotides at positions 2, 4, 6, 8, 13, 15, 17 and 19 and may further comprise at least one abasic or inverted abasic pseudo-nucleotide for example in position 11. In other embodiments, (N)x comprises 2′O Me modified ribonucleotides at positions 2, 4, 6, 8, 11, 13, 15, 17 and 19 and may further comprise at least one abasic or inverted abasic pseudo-nucleotide for example in position 12. In other embodiments, (N)x comprises 2′O Me modified ribonucleotides at positions 2, 4, 6, 8, 11, 15, 17 and 19 and may further comprise at least one abasic or inverted abasic pseudo-nucleotide for example in position 13.

In yet other embodiments (N)x comprises at least one nucleotide mismatch relative to the target gene. In certain preferred embodiments, (N)x comprises a single nucleotide mismatch on position 5, 6, or 14. In one embodiment of Structure (C), at least two nucleotides at either or both the 5′ and 3′ termini of (N′)y are joined by a 2′-5′ phosphodiester bond. In certain preferred embodiments x=y=19 or x=y=23; in (N)x the nucleotides alternate between modified ribonucleotides and unmodified ribonucleotides, each modified ribonucleotide being modified so as to have a 2′-O-methyl on its sugar and the ribonucleotide located at the middle of (N)x being unmodified; and three nucleotides at the 3′ terminus of (N′)y are joined by two 2′-5′ phosphodiester bonds (set forth herein as Structure I). In other preferred embodiments, x=y=19 or x=y=23; in (N)x the nucleotides alternate between modified ribonucleotides and unmodified ribonucleotides, each modified ribonucleotide being modified so as to have a 2′-O-methyl on its sugar and the ribonucleotide located at the middle of (N)x being unmodified; and four consecutive nucleotides at the 5′ terminus of (N′)y are joined by three 2′-5′ phosphodiester bonds. In a further embodiment, an additional nucleotide located in the middle position of (N)y may be modified with 2′-O-methyl on its sugar. In another preferred embodiment, in (N)x the nucleotides alternate between 2′-O-methyl modified ribonucleotides and unmodified ribonucleotides, and in (N′)y four consecutive nucleotides at the 5′ terminus are joined by three 2′-5′ phosphodiester bonds and the 5′ terminal nucleotide or two or three consecutive nucleotides at the 5′ terminus comprise 3′-O-methyl modifications.

In certain preferred embodiments of Structure C, x=y=19 and in (N′)y, at least one position comprises an abasic or inverted abasic pseudo-nucleotide, preferably five positions comprises an abasic or inverted abasic pseudo-nucleotides. In various embodiments, the following positions comprise an abasic or inverted abasic: positions 1 and 16-19, positions 15-19, positions 1-2 and 17-19, positions 1-3 and 18-19, positions 1-4 and 19 and positions 1-5. (N′)y may further comprise at least one LNA nucleotide.

In certain preferred embodiments of Structure C, x=y=19 and in (N′)y the nucleotide in at least one position comprises a mirror nucleotide, a deoxyribonucleotide and a nucleotide joined to an adjacent nucleotide by a 2′-5′ internucleotide bond.

In certain preferred embodiments of Structure C, x=y=19 and (N′)y comprises a mirror nucleotide. In various embodiments the mirror nucleotide is an L-DNA nucleotide. In certain embodiments the L-DNA is L-deoxyribocytidine. In some embodiments (N′)y comprises L-DNA at position 18. In other embodiments (N′)y comprises L-DNA at positions 17 and 18. In certain embodiments (N′)y comprises L-DNA substitutions at positions 2 and at one or both of positions 17 and 18. In certain embodiments (N′)y further comprises a 5′ terminal cap nucleotide such as 5′-O-methyl DNA or an abasic or inverted abasic pseudo-nucleotide as an overhang.

In yet other embodiments (N′)y comprises at least one nucleotide mismatch relative to the target gene. In certain preferred embodiments, (N′)y comprises a single nucleotide mismatch on position 6, 14, or 15.

In yet other embodiments (N′)y comprises a DNA at position 15 and L-DNA at one or both of positions 17 and 18. In that structure, position 2 may further comprise an L-DNA or an abasic pseudo-nucleotide.

Other embodiments of Structure C are envisaged wherein x=y=21 or wherein x=y=23; in these embodiments the modifications for (N′)y discussed above instead of being on positions 15, 16, 17, 18 are on positions 17, 18, 19, 20 for 21 mer and on positions 19, 20, 21, 22 for 23 mer; similarly the modifications at one or both of positions 17 and 18 are on one or both of positions 19 or 20 for the 21 mer and one or both of positions 21 and 22 for the 23 mer. All modifications in the 19 mer are similarly adjusted for the 21 and 23 mers.

According to various embodiments of Structure (C), in (N′)y 2, 3, 4, 5, 6, 7, 8, 9, 10, 11, 12, 13 or 14 consecutive ribonucleotides at the 3′ terminus are linked by 2′-5′ internucleotide linkages In one preferred embodiment, four consecutive nucleotides at the 3′ terminus of (N′)y are joined by three 2′-5′ phosphodiester bonds, wherein one or more of the 2′-5′ nucleotides which form the 2′-5′ phosphodiester bonds further comprises a 3′-O-methyl sugar modification. Preferably the 3′ terminal nucleotide of (N′)y comprises a 2′-O-methyl sugar modification. In certain preferred embodiments of Structure C, x=y=19 and in (N′)y two or more consecutive nucleotides at positions 15, 16, 17, 18 and 19 comprise a nucleotide joined to an adjacent nucleotide by a 2′-5′ internucleotide bond. In various embodiments the nucleotide forming the 2′-5′ internucleotide bond comprises a 3′ deoxyribose nucleotide or a 3′ methoxy nucleotide. In some embodiments the nucleotides at positions 17 and 18 in (N′)y are joined by a 2′-5′ internucleotide bond. In other embodiments the nucleotides at positions 16, 17, 18, 16-17, 17-18, or 16-18 in (N′)y are joined by a 2′-5′ internucleotide bond.

In certain embodiments (N′)y comprises an L-DNA at position 2 and 2′-5′ internucleotide bonds at positions 16, 17, 18, 16-17, 17-18, or 16-18. In certain embodiments (N′)y comprises 2′-5′ internucleotide bonds at positions 16, 17, 18, 16-17, 17-18, or 16-18 and a 5′ terminal cap nucleotide.

According to various embodiments of Structure (C), in (N′)y 2, 3, 4, 5, 6, 7, 8, 9, 10, 11, 12, 13 or 14 consecutive nucleotides at either terminus or 2-8 modified nucleotides at each of the 5′ and 3′ termini are independently mirror nucleotides. In some embodiments the mirror nucleotide is an L-ribonucleotide. In other embodiments the mirror nucleotide is an L-deoxyribonucleotide. The mirror nucleotide may further be modified at the sugar or base moiety or in an internucleotide linkage.

In one preferred embodiment of Structure (C), the 3′ terminal nucleotide or two or three consecutive nucleotides at the 3′ terminus of (N′)y are L-deoxyribonucleotides.

In other embodiments of Structure (C), in (N′)y 2, 3, 4, 5, 6, 7, 8, 9, 10, 11, 12, 13 or 14 consecutive ribonucleotides at either terminus or 2-8 modified nucleotides at each of the 5′ and 3′ termini are independently 2′ sugar modified nucleotides. In some embodiments the 2′ sugar modification comprises the presence of an amino, a fluoro, an alkoxy or an alkyl moiety. In certain embodiments the 2′ sugar modification comprises a methoxy moiety (2′-OMe).

In one series of preferred embodiments, three, four or five consecutive nucleotides at the 5′ terminus of (N′)y comprise the 2′-OMe modification. In another preferred embodiment, three consecutive nucleotides at the 3′ terminus of (N′)y comprise the 2′-O-methyl modification.

In some embodiments of Structure (C), in (N′)y 2, 3, 4, 5, 6, 7, 8, 9, 10, 11, 12, 13 or 14 consecutive ribonucleotides at either or 2-8 modified nucleotides at each of the 5′ and 3′ termini are independently bicyclic nucleotide. In various embodiments the bicyclic nucleotide is a locked nucleic acid (LNA). A 2′-O, 4′-C-ethylene-bridged nucleic acid (ENA) is a species of LNA (see below).

In various embodiments (N′)y comprises modified nucleotides at the 5′ terminus or at both the 3′ and 5′ termini.

In some embodiments of Structure (C), at least two nucleotides at either or both the 5′ and 3′ termini of (N′)y are joined by P-ethoxy backbone modifications. In certain preferred embodiments x=y=19 or x=y=23; in (N)x the nucleotides alternate between modified ribonucleotides and unmodified ribonucleotides, each modified ribonucleotide being modified so as to have a 2′-O-methyl on its sugar and the ribonucleotide located at the middle position of (N)x being unmodified; and four consecutive nucleotides at the 3′ terminus or at the 5′ terminus of (N′)y are joined by three P-ethoxy backbone modifications. In another preferred embodiment, three consecutive nucleotides at the 3′ terminus or at the 5′ terminus of (N′)y are joined by two P-ethoxy backbone modifications.

In some embodiments of Structure (C), in (N′)y 2, 3, 4, 5, 6, 7 or 8, consecutive ribonucleotides at each of the 5′ and 3′ termini are independently mirror nucleotides, nucleotides joined by 2′-5′ phosphodiester bond, 2′ sugar modified nucleotides or bicyclic nucleotide. In one embodiment, the modification at the 5′ and 3′ termini of (N′)y is identical. In one preferred embodiment, four consecutive nucleotides at the 5′ terminus of (N′)y are joined by three 2′-5′ phosphodiester bonds and three consecutive nucleotides at the 3′ terminus of (N′)y are joined by two 2′-5′ phosphodiester bonds. In another embodiment, the modification at the 5′ terminus of (N′)y is different from the modification at the 3′ terminus of (N′)y. In one specific embodiment, the modified nucleotides at the 5′ terminus of (N′)y are mirror nucleotides and the modified nucleotides at the 3′ terminus of (N′)y are joined by 2′-5′ phosphodiester bond. In another specific embodiment, three consecutive nucleotides at the 5′ terminus of (N′)y are LNA nucleotides and three consecutive nucleotides at the 3′ terminus of (N′)y are joined by two 2′-5′ phosphodiester bonds. In (N)x the nucleotides alternate between modified ribonucleotides and unmodified ribonucleotides, each modified ribonucleotide being modified so as to have a 2′-O-methyl on its sugar and the ribonucleotide located at the middle of (N)x being unmodified, or the ribonucleotides in (N)x being unmodified

In another embodiment of Structure (C), the present invention provides a compound wherein x=y=19 or x=y=23; in (N)x the nucleotides alternate between modified ribonucleotides and unmodified ribonucleotides, each modified ribonucleotide being modified so as to have a 2′-O-methyl on its sugar and the ribonucleotide located at the middle of (N)x being unmodified; three nucleotides at the 3′ terminus of (N′)y are joined by two 2′-5′ phosphodiester bonds and three nucleotides at the 5′ terminus of (N′)y are LNA such as ENA.

In another embodiment of Structure (C), five consecutive nucleotides at the 5′ terminus of (N′)y comprise the 2′-O-methyl sugar modification and two consecutive nucleotides at the 3′ terminus of (N′)y are L-DNA.

In yet another embodiment, the present invention provides a compound wherein x=y=19 or x=y=23; (N)x consists of unmodified ribonucleotides; three consecutive nucleotides at the 3′ terminus of (N′)y are joined by two 2′-5′ phosphodiester bonds and three consecutive nucleotides at the 5′ terminus of (N′)y are LNA such as ENA.

According to other embodiments of Structure (C), in (N′)y the 5′ or 3′ terminal nucleotide, or 2, 3, 4, 5 or 6 consecutive nucleotides at either termini or 1-4 modified nucleotides at each of the 5′ and 3′ termini are independently phosphonocarboxylate or phosphinocarboxylate nucleotides (PACE nucleotides). In some embodiments the PACE nucleotides are deoxyribonucleotides. In some preferred embodiments in (N′)y, 1 or 2 consecutive nucleotides at each of the 5′ and 3′ termini are PACE nucleotides. Examples of PACE nucleotides and analogs are disclosed in U.S. Pat. Nos. 6,693,187 and 7,067,641 both incorporated by reference.

In additional embodiments, the present invention provides a compound having Structure (D):

| | | | | |
|---|---|---|---|---|
| (D) | 5′ | (N)x-Z | 3′ | antisense strand |
| | 3′ | Z′-(N′)y | 5′ | sense strand |

wherein each of N and N′ is a nucleotide selected from an unmodified ribonucleotide, a modified ribonucleotide, an unmodified deoxyribonucleotide or a modified deoxyribonucleotide;
wherein each of (N)x and (N′)y is an oligomer in which each consecutive nucleotide is joined to the next nucleotide by a covalent bond and each of x and y is an integer between 18 and 40;
wherein (N)x comprises unmodified ribonucleotides further comprising one modified nucleotide at the 3′ terminal or penultimate position, wherein the modified nucleotide is selected from the group consisting of a bicyclic nucleotide, a 2′ sugar modified nucleotide, a mirror nucleotide, an altritol nucleotide, or a nucleotide joined to an adjacent nucleotide by an internucleotide linkage selected from a 2′-5′ phosphodiester bond, a P-alkoxy linkage or a PACE linkage;
wherein (N′)y comprises unmodified ribonucleotides further comprising one modified nucleotide at the 5′ terminal or penultimate position, wherein the modified nucleotide is selected from the group consisting of a bicyclic nucleotide, a 2′ sugar modified nucleotide, a mirror nucleotide, an altritol nucleotide, or a nucleotide joined to an adjacent nucleotide by an internucleotide linkage selected from a 2′-5′ phosphodiester bond, a P-alkoxy linkage or a PACE linkage;
wherein in each of (N)x and (N′)y modified and unmodified nucleotides are not alternating;
wherein each of Z and Z′ may be present or absent, but if present is 1-5 deoxyribonucleotides covalently attached at the 3′ terminus of any oligomer to which it is attached;
wherein the sequence of (N′)_{y} is a sequence substantially complementary to (N)x; and wherein the sequence of (N)ₓ comprises an antisense sequence having substantial identity to about 18 to about 40 consecutive ribonucleotides in the mRNA transcribed from a gene.

In one embodiment of Structure (D), x=y=19 or x=y=23; (N)x comprises unmodified ribonucleotides in which two consecutive nucleotides linked by one 2′-5′ internucleotide linkage at the 3′ terminus; and (N′)y comprises unmodified ribonucleotides in which two consecutive nucleotides linked by one 2′-5′ internucleotide linkage at the 5′ terminus.

In some embodiments, x=y=19 or x=y=23; (N)x comprises unmodified ribonucleotides in which three consecutive nucleotides at the 3′ terminus are joined together by two 2′-5′ phosphodiester bonds; and (N′)y comprises unmodified ribonucleotides in which four consecutive nucleotides at the 5′ terminus are joined together by three 2′-5′ phosphodiester bonds (set forth herein as Structure II).

According to various embodiments of Structure (D) 2, 3, 4, 5, 6, 7, 8, 9, 10, 11, 12, 13 or 14 consecutive ribonucleotides starting at the ultimate or penultimate position of the 3′ terminus of (N)x and 2, 3, 4, 5, 6, 7, 8, 9, 10, 11, 12, 13 or 14 consecutive ribonucleotides starting at the ultimate or penultimate position of the 5′ terminus of (N′)y are linked by 2′-5′ internucleotide linkages.

According to one preferred embodiment of Structure (D), four consecutive nucleotides at the 5′ terminus of (N′)y are joined by three 2′-5′ phosphodiester bonds and three consecutive nucleotides at the 3′ terminus of (N′)x are joined by two 2′-5′ phosphodiester bonds. Three nucleotides at the 5′ terminus of (N′)y and two nucleotides at the 3′ terminus of (N′)x may also comprise 3′-O-methyl modifications.

According to various embodiments of Structure (D), 2, 3, 4, 5, 6, 7, 8, 9, 10, 11, 12, 13 or 14 consecutive nucleotides starting at the ultimate or penultimate position of the 3′ terminus of (N)x and 2, 3, 4, 5, 6, 7, 8, 9, 10, 11, 12, 13 or 14 consecutive ribonucleotides starting at the ultimate or penultimate position of the 5′ terminus of (N′)y are independently mirror nucleotides. In some embodiments the mirror is an L-ribonucleotide. In other embodiments the mirror nucleotide is L-deoxyribonucleotide.

In other embodiments of Structure (D), 2, 3, 4, 5, 6, 7, 8, 9, 10, 11, 12, 13 or 14 consecutive ribonucleotides starting at the ultimate or penultimate position of the 3′ terminus of (N)x and 2, 3, 4, 5, 6, 7, 8, 9, 10, 11, 12, 13 or 14 consecutive ribonucleotides starting at the ultimate or penultimate position of the 5′ terminus of (N′)y are independently 2′ sugar modified nucleotides. In some embodiments the 2′ sugar modification comprises the presence of an amino, a fluoro, an alkoxy or an alkyl moiety. In certain embodiments the 2′ sugar modification comprises a methoxy moiety (2′-OMe).

In one preferred embodiment of Structure (D), five consecutive nucleotides at the 5′ terminus of (N′)y comprise the 2′-O-methyl modification and five consecutive nucleotides at the 3′ terminus of (N′)x comprise the 2′-O-methyl modification. In another preferred embodiment of Structure (D), ten consecutive nucleotides at the 5′ terminus of (N′)y comprise the 2′-O-methyl modification and five consecutive nucleotides at the 3′ terminus of (N′)x comprise the 2′-O-methyl modification. In another preferred embodiment of Structure (D), thirteen consecutive nucleotides at the 5′ terminus of (N′)y comprise the 2′-O-methyl modification and five consecutive nucleotides at the 3′ terminus of (N′)x comprise the 2′-O-methyl modification.

In some embodiments of Structure (D), in (N′)y 2, 3, 4, 5, 6, 7, 8, 9, 10, 11, 12, 13 or 14 consecutive ribonucleotides starting at the ultimate or penultimate position of the 3′ terminus of (N)x and 2, 3, 4, 5, 6, 7, 8, 9, 10, 11, 12, 13 or 14 consecutive ribonucleotides starting at the ultimate or penultimate position of the 5′ terminus of (N′)y are independently a bicyclic nucleotide. In various embodiments the bicyclic nucleotide is a locked nucleic acid (LNA) such as a 2′-O, 4′-C-ethylene-bridged nucleic acid (ENA).

In various embodiments of Structure (D), (N′)y comprises a modified nucleotide selected from a bicyclic nucleotide, a 2′ sugar modified nucleotide, a mirror nucleotide, an altritol nucleotide or a nucleotide joined to an adjacent nucleotide by an internucleotide linkage selected from a 2′-5′ phosphodiester bond, a P-alkoxy linkage or a PACE linkage;

In various embodiments of Structure (D), (N)x comprises a modified nucleotide selected from a bicyclic nucleotide, a 2′ sugar modified nucleotide, a mirror nucleotide, an altritol nucleotide or a nucleotide joined to an adjacent nucleotide by an internucleotide linkage selected from a 2′-5′ phosphodiester bond, a P-alkoxy linkage or a PACE linkage;

In embodiments wherein each of the 3′ and 5′ termini of the same strand comprises a modified nucleotide, the modification at the 5′ and 3′ termini is identical. In another embodiment, the modification at the 5′ terminus is different from the modification at the 3′ terminus of the same strand. In one specific embodiment, the modified nucleotides at the 5′ terminus are mirror nucleotides and the modified nucleotides at the 3′ terminus of the same strand are joined by 2′-5′ phosphodiester bond.

In one specific embodiment of Structure (D), five consecutive nucleotides at the 5′ terminus of (N′)y comprise the 2′-O-methyl modification and two consecutive nucleotides at the 3′ terminus of (N′)y are L-DNA. In addition, the compound may further comprise five consecutive 2′-O-methyl modified nucleotides at the 3′ terminus of (N′)x.

In various embodiments of Structure (D), the modified nucleotides in (N)x are different from the modified nucleotides in (N′)y. For example, the modified nucleotides in (N)x are 2′ sugar modified nucleotides and the modified nucleotides in (N′)y are nucleotides linked by 2′-5′ internucleotide linkages. In another example, the modified nucleotides in (N)x are mirror nucleotides and the modified nucleotides in (N′)y are nucleotides linked by 2′-5′ internucleotide linkages. In another example, the modified nucleotides in (N)x are nucleotides linked by 2′-5′ internucleotide linkages and the modified nucleotides in (N′)y are mirror nucleotides.

In additional embodiments, the present invention provides a compound having Structure (E):

| | | | | |
|---|---|---|---|---|
| (E) | 5′ | (N)x-Z | 3′ | antisense strand |
| | 3′ | Z′-(N′)y | 5′ | sense strand |

wherein each of N and N′ is a nucleotide selected from an unmodified ribonucleotide, a modified ribonucleotide, an unmodified deoxyribonucleotide or a modified deoxyribonucleotide;
wherein each of (N)x and (N′)y is an oligomer in which each consecutive nucleotide is joined to the next nucleotide by a covalent bond and each of x and y is an integer between 18 and 40;
wherein (N)x comprises unmodified ribonucleotides further comprising one modified nucleotide at the 5′ terminal or penultimate position, wherein the modified nucleotide is selected from the group consisting of a bicyclic nucleotide, a 2′ sugar modified nucleotide, a mirror nucleotide, an altritol nucleotide, or a nucleotide joined to an adjacent nucleotide by an internucleotide linkage selected from a 2′-5′ phosphodiester bond, a P-alkoxy linkage or a PACE linkage;
wherein (N′)y comprises unmodified ribonucleotides further comprising one modified nucleotide at the 3′ terminal or penultimate position, wherein the modified nucleotide is selected from the group consisting of a bicyclic nucleotide, a 2′ sugar modified nucleotide, a mirror nucleotide, an altritol nucleotide, or a nucleotide joined to an adjacent nucleotide by an internucleotide linkage selected from a 2′-5′ phosphodiester bond, a P-alkoxy linkage or a PACE linkage;
wherein in each of (N)x and (N′)y modified and unmodified nucleotides are not alternating;
wherein each of Z and Z′ may be present or absent, but if present is 1-5 deoxyribonucleotides covalently attached at the 3′ terminus of any oligomer to which it is attached;
wherein the sequence of (N′)y is a sequence substantially complementary to (N)x; and wherein the sequence of (N)ₓ comprises an antisense sequence having substantial identity to about 18 to about 40 consecutive ribonucleotides in the mRNA transcribed from a gene.

In certain preferred embodiments the ultimate nucleotide at the 5′ terminus of (N)x is unmodified.

According to various embodiments of Structure (E) 2, 3, 4, 5, 6, 7, 8, 9, 10, 11, 12, 13 or 14 consecutive ribonucleotides starting at the ultimate or penultimate position of the 5′ terminus of (N)x, preferably starting at the 5′ penultimate position, and 2, 3, 4, 5, 6, 7, 8, 9, 10, 11, 12, 13 or 14 consecutive ribonucleotides starting at the ultimate or penultimate position of the 3′ terminus of (N′)y are linked by 2′-5′ internucleotide linkages.

According to various embodiments of Structure (E), 2, 3, 4, 5, 6, 7, 8, 9, 10, 11, 12, 13 or 14 consecutive nucleotides starting at the ultimate or penultimate position of the 5′ terminus of (N)x, preferably starting at the 5′ penultimate position, and 2, 3, 4, 5, 6, 7, 8, 9, 10, 11, 12, 13 or 14 consecutive nucleotides starting at the ultimate or penultimate position of the 3′ terminus of (N′)y are independently mirror nucleotides. In some embodiments the mirror is an L-ribonucleotide. In other embodiments the mirror nucleotide is L-deoxyribonucleotide.

In other embodiments of Structure (E), 2, 3, 4, 5, 6, 7, 8, 9, 10, 11, 12, 13 or 14 consecutive ribonucleotides starting at the ultimate or penultimate position of the 5′ terminus of (N)x, preferably starting at the 5′ penultimate position, and 2, 3, 4, 5, 6, 7, 8, 9, 10, 11, 12, 13 or 14 consecutive ribonucleotides starting at the ultimate or penultimate position of the 3′ terminus of (N′)y are independently 2′ sugar modified nucleotides. In some embodiments the 2′ sugar modification comprises the presence of an amino, a fluoro, an alkoxy or an alkyl moiety. In certain embodiments the 2′ sugar modification comprises a methoxy moiety (2′-OMe).

In some embodiments of Structure (E), in (N′)y 2, 3, 4, 5, 6, 7, 8, 9, 10, 11, 12, 13 or 14 consecutive ribonucleotides starting at the ultimate or penultimate position of the 5′ terminus of (N)x, preferably starting at the 5′ penultimate position, and 2, 3, 4, 5, 6, 7, 8, 9, 10, 11, 12, 13 or 14 consecutive ribonucleotides starting at the ultimate or penultimate position of the 3′ terminus of (N′)y are independently a bicyclic nucleotide. In various embodiments the bicyclic nucleotide is a locked nucleic acid (LNA) such as a 2′-O, 4′-C-ethylene-bridged nucleic acid (ENA).

In various embodiments of Structure (E), (N′)y comprises modified nucleotides selected from a bicyclic nucleotide, a 2′ sugar modified nucleotide, a mirror nucleotide, an altritol nucleotide, a nucleotide joined to an adjacent nucleotide by a P-alkoxy backbone modification or a nucleotide joined to an adjacent nucleotide by an internucleotide linkage selected from a 2′-5′ phosphodiester bond, a P-alkoxy linkage or a PACE linkage

at the 3′ terminus or at each of the 3′ and 5′ termini.

In various embodiments of Structure (E), (N)x comprises a modified nucleotide selected from a bicyclic nucleotide, a 2′ sugar modified nucleotide, a mirror nucleotide, an altritol nucleotide, or a nucleotide joined to an adjacent nucleotide by an internucleotide linkage selected from a 2′-5′ phosphodiester bond, a P-alkoxy linkage or a PACE linkage at the 5′ terminus or at each of the 3′ and 5′ termini.

In one embodiment where both 3′ and 5′ termini of the same strand comprise a modified nucleotide, the modification at the 5′ and 3′ termini is identical. In another embodiment, the modification at the 5′ terminus is different from the modification at the 3′ terminus of the same strand. In one specific embodiment, the modified nucleotides at the 5′ terminus are mirror nucleotides and the modified nucleotides at the 3′ terminus of the same strand are joined by 2′-5′ phosphodiester bond.

In various embodiments of Structure (E), the modified nucleotides in (N)x are different from the modified nucleotides in (N′)y. For example, the modified nucleotides in (N)x are 2′ sugar modified nucleotides and the modified nucleotides in (N′)y are nucleotides linked by 2′-5′ internucleotide linkages. In another example, the modified nucleotides in (N)x are mirror nucleotides and the modified nucleotides in (N′)y are nucleotides linked by 2′-5′ internucleotide linkages. In another example, the modified nucleotides in (N)x are nucleotides linked by 2′-5′ internucleotide linkages and the modified nucleotides in (N′)y are mirror nucleotides.

In additional embodiments, the present invention provides a compound having Structure (F):

| | |
|---|---|
| (F) | 5′ (N)x-Z 3′ antisense strand |
| | 3′ Z′-(N′)y 5′ sense strand |

wherein each of N and N′ is a nucleotide selected from an unmodified ribonucleotide, a modified ribonucleotide, an unmodified deoxyribonucleotide or a modified deoxyribonucleotide;
wherein each of (N)x and (N′)y is an oligomer in which each consecutive nucleotide is joined to the next nucleotide by a covalent bond and each of x and y is an integer between 18 and 40;
wherein each of (N)x and (N′)y comprise unmodified ribonucleotides in which each of (N)x and (N′)y independently comprise one modified nucleotide at the 3′ terminal or penultimate position wherein the modified nucleotide is selected from the group consisting of a bicyclic nucleotide, a 2′ sugar modified nucleotide, a mirror nucleotide, a nucleotide joined to an adjacent nucleotide by a P-alkoxy backbone modification or a nucleotide joined to an adjacent nucleotide by a 2′-5′ phosphodiester bond;
wherein in each of (N)x and (N′)y modified and unmodified nucleotides are not alternating;
wherein each of Z and Z′ may be present or absent, but if present is 1-5 deoxyribonucleotides covalently attached at the 3′ terminus of any oligomer to which it is attached;
wherein the sequence of (N′)_{y} is a sequence substantially complementary to (N)x; and wherein the sequence of (N)ₓ comprises an antisense sequence having substantial identity to about 18 to about 40 consecutive ribonucleotides in the mRNA transcribed from a gene.

In some embodiments of Structure (F), x=y=19 or x=y=23; (N′)y comprises unmodified ribonucleotides in which two consecutive nucleotides at the 3′ terminus comprises two consecutive mirror deoxyribonucleotides; and (N)x comprises unmodified ribonucleotides in which one nucleotide at the 3′ terminus comprises a mirror deoxyribonucleotide (set forth as Structure III).

According to various embodiments of Structure (F) 2, 3, 4, 5, 6, 7, 8, 9, 10, 11, 12, 13 or 14 consecutive ribonucleotides independently beginning at the ultimate or penultimate position of the 3′ termini of (N)x and (N′)y are linked by 2′-5′ internucleotide linkages.

According to one preferred embodiment of Structure (F), three consecutive nucleotides at the 3′ terminus of (N′)y are joined by two 2′-5′ phosphodiester bonds and three consecutive nucleotides at the 3′ terminus of (N′)x are joined by two 2′-5′ phosphodiester bonds.

According to various embodiments of Structure (F), 2, 3, 4, 5, 6, 7, 8, 9, 10, 11, 12, 13 or 14 consecutive nucleotides independently beginning at the ultimate or penultimate position of the 3′ termini of (N)x and (N′)y are independently mirror nucleotides. In some embodiments the mirror nucleotide is an L-ribonucleotide. In other embodiments the mirror nucleotide is an L-deoxyribonucleotide.

In other embodiments of Structure (F), 2, 3, 4, 5, 6, 7, 8, 9, 10, 11, 12, 13 or 14 consecutive ribonucleotides independently beginning at the ultimate or penultimate position of the 3′ termini of (N)x and (N′)y are independently 2′ sugar modified nucleotides. In some embodiments the 2′ sugar modification comprises the presence of an amino, a fluoro, an alkoxy or an alkyl moiety. In certain embodiments the 2′ sugar modification comprises a methoxy moiety (2′-OMe).

In some embodiments of Structure (F), 2, 3, 4, 5, 6, 7, 8, 9, 10, 11, 12, 13 or 14 consecutive ribonucleotides independently beginning at the ultimate or penultimate position of the 3′ termini of (N)x and (N′)y are independently a bicyclic nucleotide. In various embodiments the bicyclic nucleotide is a locked nucleic acid (LNA) such as a 2′-O, 4′-C-ethylene-bridged nucleic acid (ENA).

In various embodiments of Structure (F), (N′)y comprises a modified nucleotide selected from a bicyclic nucleotide, a 2′ sugar modified nucleotide, a mirror nucleotide, an altritol nucleotide, or a nucleotide joined to an adjacent nucleotide by an internucleotide linkage selected from a 2′-5′ phosphodiester bond, a P-alkoxy linkage or a PACE linkage at the 3′ terminus or at both the 3′ and 5′ termini.

In various embodiments of Structure (F), (N)x comprises a modified nucleotide selected from a bicyclic nucleotide, a 2′ sugar modified nucleotide, a mirror nucleotide, an altritol nucleotide, or a nucleotide joined to an adjacent nucleotide by an internucleotide linkage selected from a 2′-5′ phosphodiester bond, a P-alkoxy linkage or a PACE linkage at the 3′ terminus or at each of the 3′ and 5′ termini.

In one embodiment where each of 3′ and 5′ termini of the same strand comprise a modified nucleotide, the modification at the 5′ and 3′ termini is identical. In another embodiment, the modification at the 5′ terminus is different from the modification at the 3′ terminus of the same strand. In one specific embodiment, the modified nucleotides at the 5′ terminus are mirror nucleotides and the modified nucleotides at the 3′ terminus of the same strand are joined by 2′-5′ phosphodiester bond.

In various embodiments of Structure (F), the modified nucleotides in (N)x are different from the modified nucleotides in (N′)y. For example, the modified nucleotides in (N)x are 2′ sugar modified nucleotides and the modified nucleotides in (N′)y are nucleotides linked by 2′-5′ internucleotide linkages. In another example, the modified nucleotides in (N)x are mirror nucleotides and the modified nucleotides in (N′)y are nucleotides linked by 2′-5′ internucleotide linkages. In another example, the modified nucleotides in (N)x are nucleotides linked by 2′-5′ internucleotide linkages and the modified nucleotides in (N′)y are mirror nucleotides.

In additional embodiments, the present invention provides a compound having Structure (G):

| | |
|---|---|
| (G) | 5′ (N)x-Z 3′ antisense strand |
| | 3′ Z′-(N′)y 5' sense strand |

wherein each of N and N′ is a nucleotide selected from an unmodified ribonucleotide, a modified ribonucleotide, an unmodified deoxyribonucleotide or a modified deoxyribonucleotide;
wherein each of (N)x and (N′)y is an oligomer in which each consecutive nucleotide is joined to the next nucleotide by a covalent bond and each of x and y is an integer between 18 and 40;
wherein each of (N)x and (N′)y comprise unmodified ribonucleotides in which each of (N)x and (N′)y independently comprise one modified nucleotide at the 5′ terminal or penultimate position wherein the modified nucleotide is selected from the group consisting of a bicyclic nucleotide, a 2′ sugar modified nucleotide, a mirror nucleotide, a nucleotide joined to an adjacent nucleotide by a P-alkoxy backbone modification or a nucleotide joined to an adjacent nucleotide by a 2′-5′ phosphodiester bond;
wherein for (N)x the modified nucleotide is preferably at penultimate position of the 5′ terminal;
wherein in each of (N)x and (N′)y modified and unmodified nucleotides are not alternating;
wherein each of Z and Z′ may be present or absent, but if present is 1-5 deoxyribonucleotides covalently attached at the 3′ terminus of any oligomer to which it is attached;
wherein the sequence of (N′)_{y} is a sequence substantially complementary to (N)x; and wherein the sequence of (N)ₓ comprises an antisense sequence having substantial identity to about 18 to about 40 consecutive ribonucleotides in the mRNA transcribed from a gene.

In some embodiments of Structure (G), x=y=19 or x=y=23.

According to various embodiments of Structure (G) 2, 3, 4, 5, 6, 7, 8, 9, 10, 11, 12, 13 or 14 consecutive ribonucleotides independently beginning at the ultimate or penultimate position of the 5′ termini of (N)x and (N′)y are linked by 2′-5′ internucleotide linkages. For (N)x the modified nucleotides preferably starting at the penultimate position of the 5′ terminal.

According to various embodiments of Structure (G), 2, 3, 4, 5, 6, 7, 8, 9, 10, 11, 12, 13 or 14 consecutive nucleotides independently beginning at the ultimate or penultimate position of the 5′ termini of (N)x and (N′)y are independently mirror nucleotides. In some embodiments the mirror nucleotide is an L-ribonucleotide. In other embodiments the mirror nucleotide is an L-deoxyribonucleotide. For (N)x the modified nucleotides preferably starting at the penultimate position of the 5′ terminal.

In other embodiments of Structure (G), 2, 3, 4, 5, 6, 7, 8, 9, 10, 11, 12, 13 or 14 consecutive ribonucleotides independently beginning at the ultimate or penultimate position of the 5′ termini of (N)x and (N′)y are independently 2′ sugar modified nucleotides. In some embodiments the 2′ sugar modification comprises the presence of an amino, a fluoro, an alkoxy or an alkyl moiety. In certain embodiments the 2′ sugar modification comprises a methoxy moiety (2′-OMe). In some preferred embodiments the consecutive modified nucleotides preferably begin at the penultimate position of the 5′ terminus of (N)x.

In one preferred embodiment of Structure (G), five consecutive ribonucleotides at the 5′ terminus of (N′)y comprise a 2′-O-methyl modification and one ribonucleotide at the 5′ penultimate position of (N′)x comprises a 2′-O-methyl modification. In another preferred embodiment of Structure (G), five consecutive ribonucleotides at the 5′ terminus of (N′)y comprise a 2′-O-methyl modification and two consecutive ribonucleotides at the 5′ terminal position of (N′)x comprise a 2′-O-methyl modification.

In some embodiments of Structure (G), 2, 3, 4, 5, 6, 7, 8, 9, 10, 11, 12, 13 or 14 consecutive ribonucleotides independently beginning at the ultimate or penultimate position of the 5′ termini of (N)x and (N′)y are bicyclic nucleotides. In various embodiments the bicyclic nucleotide is a locked nucleic acid (LNA) such as a 2′-O, 4′-C-ethylene-bridged nucleic acid (ENA). In some preferred embodiments the consecutive modified nucleotides preferably begin at the penultimate position of the 5′ terminus of (N)x.

In various embodiments of Structure (G), (N′)y comprises a modified nucleotide selected from a bicyclic nucleotide, a 2′ sugar modified nucleotide, a mirror nucleotide, an altritol nucleotide, or a nucleotide joined to an adjacent nucleotide by an internucleotide linkage selected from a 2′-5′ phosphodiester bond, a P-alkoxy linkage or a PACE linkage at the 5′ terminus or at each of the 3′ and 5′ termini.

In various embodiments of Structure (G), (N)x comprises a modified nucleotide selected from a bicyclic nucleotide, a 2′ sugar modified nucleotide, a mirror nucleotide, an altritol nucleotide, or a nucleotide joined to an adjacent nucleotide by an internucleotide linkage selected from a 2′-5′ phosphodiester bond, a P-alkoxy linkage or a PACE linkage at the 5′ terminus or at each of the 3′ and 5′ termini.

In one embodiment where each of 3′ and 5′ termini of the same strand comprise a modified nucleotide, the modification at the 5′ and 3′ termini is identical. In another embodiment, the modification at the 5′ terminus is different from the modification at the 3′ terminus of the same strand. In one specific embodiment, the modified nucleotides at the 5′ terminus are mirror nucleotides and the modified nucleotides at the 3′ terminus of the same strand are joined by 2′-5′ phosphodiester bond. In various embodiments of Structure (G), the modified nucleotides in (N)x are different from the modified nucleotides in (N′)y. For example, the modified nucleotides in (N)x are 2′ sugar modified nucleotides and the modified nucleotides in (N′)y are nucleotides linked by 2′-5′ internucleotide linkages. In another example, the modified nucleotides in (N)x are mirror nucleotides and the modified nucleotides in (N′)y are nucleotides linked by 2′-5′ internucleotide linkages. In another example, the modified nucleotides in (N)x are nucleotides linked by 2′-5′ internucleotide linkages and the modified nucleotides in (N′)y are mirror nucleotides.

In additional embodiments, the present invention provides a compound having Structure (H):

| | |
|---|---|
| (H) | 5′ (N)x-Z 3′ antisense strand |
| | 3′ Z′-(N′)y 5′ sense strand |

wherein each of N and N′ is a nucleotide selected from an unmodified ribonucleotide, a modified ribonucleotide, an unmodified deoxyribonucleotide or a modified deoxyribonucleotide;
wherein each of (N)x and (N′)y is an oligomer in which each consecutive nucleotide is joined to the next nucleotide by a covalent bond and each of x and y is an integer between 18 and 40;
wherein (N)x comprises unmodified ribonucleotides further comprising one modified nucleotide at the 3′ terminal or penultimate position or the 5′ terminal or penultimate position, wherein the modified nucleotide is selected from the group consisting of a bicyclic nucleotide, a 2′ sugar modified nucleotide, a mirror nucleotide, an altritol nucleotide, or a nucleotide joined to an adjacent nucleotide by an internucleotide linkage selected from a 2′-5′ phosphodiester bond, a P-alkoxy linkage or a PACE linkage;
wherein (N′)y comprises unmodified ribonucleotides further comprising one modified nucleotide at an internal position, wherein the modified nucleotide is selected from the group consisting of a bicyclic nucleotide, a 2′ sugar modified nucleotide, a mirror nucleotide, an altritol nucleotide, or a nucleotide joined to an adjacent nucleotide by an internucleotide linkage selected from a 2′-5′ phosphodiester bond, a P-alkoxy linkage or a PACE linkage;
wherein in each of (N)x and (N′)y modified and unmodified nucleotides are not alternating;
wherein each of Z and Z′ may be present or absent, but if present is 1-5 deoxyribonucleotides covalently attached at the 3′ terminus of any oligomer to which it is attached;
wherein the sequence of (N′)_{y} is a sequence substantially complementary to (N)x; and wherein the sequence of (N)ₓ comprises an antisense sequence having substantial identity to about 18 to about 40 consecutive ribonucleotides in the mRNA transcribed from a gene.

In one embodiment of Structure (H), 2, 3, 4, 5, 6, 7, 8, 9, 10, 11, 12, 13 or 14 consecutive ribonucleotides independently beginning at the ultimate or penultimate position of the 3′ terminus or the 5′ terminus or both termini of (N)x are independently 2′ sugar modified nucleotides, bicyclic nucleotides, mirror nucleotides, altritol nucleotides or nucleotides joined to an adjacent nucleotide by a 2′-5′ phosphodiester bond and 2, 3, 4, 5, 6, 7, 8, 9, 10, 11, 12, 13 or 14 consecutive internal ribonucleotides in (N′)y are independently 2′ sugar modified nucleotides, bicyclic nucleotides, mirror nucleotides, altritol nucleotides or nucleotides joined to an adjacent nucleotide by a 2′-5′ phosphodiester bond. In some embodiments the 2′ sugar modification comprises the presence of an amino, a fluoro, an alkoxy or an alkyl moiety. In certain embodiments the 2′ sugar modification comprises a methoxy moiety (2′-OMe).

In another embodiment of Structure (H), 2, 3, 4, 5, 6, 7, 8, 9, 10, 11, 12, 13 or 14 consecutive ribonucleotides independently beginning at the ultimate or penultimate position of the 3′ terminus or the 5′ terminus or 2-8 consecutive nucleotides at each of 5′ and 3′ termini of (N′)y are independently 2′ sugar modified nucleotides, bicyclic nucleotides, mirror nucleotides, altritol nucleotides or nucleotides joined to an adjacent nucleotide by a 2′-5′ phosphodiester bond, and 2, 3, 4, 5, 6, 7, 8, 9, 10, 11, 12, 13 or 14 consecutive internal ribonucleotides in (N)x are independently 2′ sugar modified nucleotides, bicyclic nucleotides, mirror nucleotides, altritol nucleotides or nucleotides joined to an adjacent nucleotide by a 2′-5′ phosphodiester bond.

In one embodiment wherein each of 3′ and 5′ termini of the same strand comprises a modified nucleotide, the modification at the 5′ and 3′ termini is identical. In another embodiment, the modification at the 5′ terminus is different from the modification at the 3′ terminus of the same strand. In one specific embodiment, the modified nucleotides at the 5′ terminus are mirror nucleotides and the modified nucleotides at the 3′ terminus of the same strand are joined by 2′-5′ phosphodiester bond.

In various embodiments of Structure (H), the modified nucleotides in (N)x are different from the modified nucleotides in (N′)y. For example, the modified nucleotides in (N)x are 2′ sugar modified nucleotides and the modified nucleotides in (N′)y are nucleotides linked by 2′-5′ internucleotide linkages. In another example, the modified nucleotides in (N)x are mirror nucleotides and the modified nucleotides in (N′)y are nucleotides linked by 2′-5′ internucleotide linkages. In another example, the modified nucleotides in (N)x are nucleotides linked by 2′-5′ internucleotide linkages and the modified nucleotides in (N′)y are mirror nucleotides.

In one preferred embodiment of Structure (H), x=y=19; three consecutive ribonucleotides at the 9-11 nucleotide positions 9-11 of (N′)y comprise 2′-O-methyl modification and five consecutive ribonucleotides at the 3′ terminal position of (N′)x comprise 2′-O-methyl modification.

For all the above Structures (A)-(H), in various embodiments x=y and each of x and y is 19, 20, 21, 22 or 23. In certain embodiments, x=y=19. In yet other embodiments x=y=23. In additional embodiments the compound comprises 2′ modified ribonucleotides in alternating positions wherein each N at the 5′ and 3′ termini of (N)x are modified in their sugar residues and the middle ribonucleotide is not modified, e.g. ribonucleotide in position 10 in a 19-mer strand, position 11 in a 21 mer and position 12 in a 23-mer strand.

In some embodiments where x=y=21 or x=y=23 the position of modifications in the 19 mer are adjusted for the 21 and 23 mers with the proviso that the middle nucleotide of the antisense strand is preferably not modified.

In some embodiments, neither (N)x nor (N′)y are phosphorylated at the 3′ and 5′ termini. In other embodiments either or both (N)x and (N′)y are phosphorylated at the 3′ termini. In yet another embodiment, either or both (N)x and (N′)y are phosphorylated at the 3′ termini using non-cleavable phosphate groups. In yet another embodiment, either or both (N)x and (N′)y are phosphorylated at the terminal 2′ termini position using cleavable or non-cleavable phosphate groups. These particular siRNA compounds are also blunt ended and are non-phosphorylated at the termini; however, comparative experiments have shown that siRNA compounds phosphorylated at one or both of the 3′-termini have similar activity in vivo compared to the non-phosphorylated compounds.

In certain embodiments for all the above-mentioned Structures, the compound is blunt ended, for example wherein both Z and Z′ are absent. In an alternative embodiment, the compound comprises at least one 3′ overhang, wherein at least one of Z or Z′ is present. Z and Z′ independently comprises one or more covalently linked modified or non-modified nucleotides, for example inverted dT or dA; dT, LNA, mirror nucleotide and the like. In some embodiments each of Z and Z′ are independently selected from dT and dTdT. siRNA in which Z and/or Z′ is present have similar in vitro and or in vivo activity and stability as siRNA in which Z and Z′ are absent.

In certain embodiments for all the above-mentioned Structures, the compound comprises one or more phosphonocarboxylate and/or phosphinocarboxylate nucleotides (PACE nucleotides). In some embodiments the PACE nucleotides are deoxyribonucleotides and the phosphinocarboxylate nucleotides are phosphinoacetate nucleotides. Examples of PACE nucleotides and analogs are disclosed in U.S. Pat. Nos. 6,693,187 and 7,067,641, both incorporated herein by reference.

In certain embodiments for all the above-mentioned Structures, the compound comprises one or more locked nucleic acids (LNA) also defined as bridged nucleic acids or bicyclic nucleotides. Preferred locked nucleic acids are 2′-O, 4′-C-ethylene nucleosides (ENA) or 2′-O, 4′-C-methylene nucleosides. Other examples of LNA and ENA nucleotides are disclosed in WO 98/39352, WO 00/47599 and WO 99/14226, all incorporated herein by reference.

In certain embodiments for all the above-mentioned Structures, the compound comprises one or more altritol monomers (nucleotides), also defined as 1,5 anhydro-2-deoxy-D-altrito-hexitol (see for example, Allart, et al., 1998. Nucleosides & Nucleotides 17:1523-1526; Herdewijn et al., 1999. Nucleosides & Nucleotides 18:1371-1376; Fisher et al., 2007, NAR 35(4):1064-1074; all incorporated herein by reference).

The present invention explicitly excludes compounds in which each of N and/or N′ is a deoxyribonucleotide (D-A, D-C, D-G, D-T). In certain embodiments (N)x and (N′)y may comprise independently 1, 2, 3, 4, 5, 6, 7, 8, 9 or more deoxyribonucleotides. In certain embodiments the present invention provides a compound wherein each of N is an unmodified ribonucleotide and the 3′ terminal nucleotide or 2, 3, 4, 5, 6, 7, 8, 9, 10, 11, 12, 13 or 14 consecutive nucleotides at the 3′ terminus of (N′)y are deoxyribonucleotides. In yet other embodiments each of N is an unmodified ribonucleotide and the 5′ terminal nucleotide or 2, 3, 4, 5, 6, 7, 8, 9, 10, 11, 12, 13 or 14 consecutive nucleotides at the 5′ terminus of (N′)y are deoxyribonucleotides. In further embodiments the 5′ terminal nucleotide or 2, 3, 4, 5, 6, 7, 8, or 9 consecutive nucleotides at the 5′ terminus and 1, 2, 3, 4, 5, or 6 consecutive nucleotides at the 3′ termini of (N)x are deoxyribonucleotides and each of N′ is an unmodified ribonucleotide. In yet further embodiments (N)x comprises unmodified ribonucleotides and 1 or 2, 3 or 4 consecutive deoxyribonucleotides independently at each of the 5′ and 3′ termini and 1 or 2, 3, 4, 5 or 6 consecutive deoxyribonucleotides in internal positions; and each of N′ is an unmodified ribonucleotide. In certain embodiments the 3′ terminal nucleotide or 2, 3, 4, 5, 6, 7, 8, 9, 10, 11, 12 13 or 14 consecutive nucleotides at the 3′ terminus of (N′)y and the terminal 5′ nucleotide or 2, 3, 4, 5, 6, 7, 8, 9, 10, 11, 12 13 or 14 consecutive nucleotides at the 5′ terminus of (N)x are deoxyribonucleotides. The present invention excludes compounds in which each of N and/or N′ is a deoxyribonucleotide. In some embodiments the 5′ terminal nucleotide of N or 2 or 3 consecutive of N and 1,2, or 3 of N′ is a deoxyribonucleotide. Certain examples of active DNA/RNA siRNA chimeras are disclosed in US patent publication 2005/0004064, and Ui-Tei, 2008 (NAR 36(7):2136-2151) incorporated herein by reference in their entirety.

Unless otherwise indicated, in preferred embodiments of the structures discussed herein the covalent bond between each consecutive N or N′ is a phosphodiester bond.

An additional novel molecule provided by the present invention is an oligonucleotide comprising consecutive nucleotides wherein a first segment of such nucleotides encode a first inhibitory RNA molecule, a second segment of such nucleotides encode a second inhibitory RNA molecule, and a third segment of such nucleotides encode a third inhibitory RNA molecule. Each of the first, the second and the third segment may comprise one strand of a double stranded RNA and the first, second and third segments may be joined together by a linker. Further, the oligonucleotide may comprise three double stranded segments joined together by one or more linker.

Thus, one molecule provided by the present invention is an oligonucleotide comprising consecutive nucleotides which encode three inhibitory RNA molecules; said oligonucleotide may possess a triple stranded structure, such that three double stranded arms are linked together by one or more linker, such as any of the linkers presented hereinabove. This molecule forms a “star”-like structure, and may also be referred to herein as RNAstar. Such structures are disclosed in PCT patent publication WO 2007/091269, assigned to the assignee of the present invention and hereby incorporated by reference in its entirety.

A covalent bond refers to an internucleotide linkage linking one nucleotide monomer to an adjacent nucleotide monomer. A covalent bond includes for example, a phosphodiester bond, a phosphorothioate bond, a P-alkoxy bond, a P-carboxy bond and the like. The normal internucleoside linkage of RNA and DNA is a 3′ to 5′ phosphodiester linkage. In certain preferred embodiments a covalent bond is a phosphodiester bond. Covalent bond encompasses non-phosphorous-containing internucleoside linkages, such as those disclosed in WO 2004/041924 inter alia. Unless otherwise indicated, in preferred embodiments of the structures discussed herein the covalent bond between each consecutive N or N′ is a phosphodiester bond.

For all of the structures above, in some embodiments the oligonucleotide sequence of (N)x is fully complementary to the oligonucleotide sequence of (N′)y. In other embodiments (N)x and (N′)y are substantially complementary. In certain embodiments (N)x is fully complementary to a target sequence. In other embodiments (N)x is substantially complementary to a target sequence.

In some embodiments, neither (N)x nor (N′)y are phosphorylated at the 3′ and 5′ termini. In other embodiments either or both (N)x and (N′)y are phosphorylated at the 3′ termini (3′ Pi). In yet another embodiment, either or both (N)x and (N′)y are phosphorylated at the 3′ termini with non-cleavable phosphate groups. In yet another embodiment, either or both (N)x and (N′)y are phosphorylated at the terminal 2′ termini position using cleavable or non-cleavable phosphate groups. Further, the inhibitory nucleic acid molecules of the present invention may comprise one or more gaps and/or one or more nicks and/or one or more mismatches. Without wishing to be bound by theory, gaps, nicks and mismatches have the advantage of partially destabilizing the nucleic acid/siRNA, so that it may be more easily processed by endogenous cellular machinery such as DICER, DROSHA or RISC into its inhibitory components.

In the context of the present invention, a gap in a nucleic acid refers to the absence of one or more internal nucleotides in one strand, while a nick in a nucleic acid refers to the absence of an internucleotide linkage between two adjacent nucleotides in one strand. Any of the molecules of the present invention may contain one or more gaps and/or one or more nicks.

In additional embodiments the present invention provides a compound having Structure (I) set forth below:

| | |
|---|---|
| (I) | 5′ (N)x-Z 3′ (antisense strand) |
| | 3′ Z′-(N′)y-z″ 5′ (sense strand) |

wherein each of N and N′ is a ribonucleotide which may be unmodified or modified, or an unconventional moiety;
wherein each of (N)x and (N′)y is an oligonucleotide in which each consecutive N or N′ is joined to the next N or N′ by a covalent bond;
wherein Z and Z′ may be present or absent, but if present is independently 1-5 consecutive nucleotides covalently attached at the 3′ terminus of the strand in which it is present;
wherein z″ may be present or absent, but if present is a capping moiety covalently attached at the 5′ terminus of (N′)y;
wherein each of x and y are independently 18 to 27;
wherein (N)x comprises modified and unmodified ribonucleotides, each modified ribonucleotide having a 2′-O-methyl on its sugar, wherein N at the 3′ terminus of (N)x is a modified ribonucleotide, (N)x comprises at least five alternating modified ribonucleotides beginning at the 3′ end and at least nine modified ribonucleotides in total and each remaining N is an unmodified ribonucleotide;
wherein in (N′)y at least one unconventional moiety is present, which unconventional moiety may be an abasic ribose moiety, an abasic deoxyribose moiety, a modified or unmodified deoxyribonucleotide, a mirror nucleotide, and a nucleotide joined to an adjacent nucleotide by a 2′-5′ internucleotide phosphate bond; and
wherein the sequence of (N)x is substantially complementary to the sequence of (N′)y; and the sequence of (N′)y is substantially identical to the sequence of an mRNA encoded by a target gene.

In some embodiments x=y=19. In other embodiments x=y=23. In some embodiments the at least one unconventional moiety is present at positions 15, 16, 17, or 18 in (N′)y. In some embodiments the unconventional moiety is selected from a mirror nucleotide, an abasic ribose moiety and an abasic deoxyribose moiety. In some preferred embodiments the unconventional moiety is a mirror nucleotide, preferably an L-DNA moiety. In some embodiments an L-DNA moiety is present at position 17, position 18 or positions 17 and 18.

In other embodiments the unconventional moiety is an abasic moiety. In various embodiments (N′)y comprises at least five abasic ribose moieties or abasic deoxyribose moieties.

In yet other embodiments (N′)y comprises at least five abasic ribose moieties or abasic deoxyribose moieties and at least one of N′ is an LNA.

In some embodiments (N)x comprises nine alternating modified ribonucleotides. In other embodiments of Structure (I) (N)x comprises nine alternating modified ribonucleotides further comprising a 2′O modified nucleotide at position 2. In some embodiments (N)x comprises 2′O Me modified ribonucleotides at the odd numbered positions 1, 3, 5, 7, 9, 11, 13, 15, 17, 19. In other embodiments (N)x further comprises a 2′O Me modified ribonucleotide at one or both of positions 2 and 18. In yet other embodiments (N)x comprises 2′O Me modified ribonucleotides at positions 2, 4, 6, 8, 11, 13, 15, 17, 19.

In various embodiments z″ is present and is selected from an abasic ribose moiety, a deoxyribose moiety; an inverted abasic ribose moiety, a deoxyribose moiety; C6-amino-Pi; a mirror nucleotide.

In another aspect the present invention provides a compound having Structure (J) set forth below:

| | |
|---|---|
| (J) | 5′ (N)x-Z 3′ (antisense strand) |
| | 3′ Z′-(N′)y-z″ 5′ (sense strand) |

wherein each of N and N′ is a ribonucleotide which may be unmodified or modified, or an unconventional moiety;
wherein each of (N)x and (N′)y is an oligonucleotide in which each consecutive N or N′ is joined to the next N or N′ by a covalent bond;
wherein Z and Z′ may be present or absent, but if present is independently 1-5 consecutive nucleotides covalently attached at the 3′ terminus of the strand in which it is present;
wherein z″ may be present or absent but if present is a capping moiety covalently attached at the 5′ terminus of (N′)y;
wherein each of x and y are independently 18 to 27;
wherein (N)x comprises modified or unmodified ribonucleotides, and optionally at least one unconventional moiety;
wherein in (N′)y at least one unconventional moiety is present, which unconventional moiety may be an abasic ribose moiety, an abasic deoxyribose moiety, a modified or unmodified deoxyribonucleotide, a mirror nucleotide, a non-base pairing nucleotide analog or a nucleotide joined to an adjacent nucleotide by a 2′-5′ internucleotide phosphate bond; and
wherein the sequence of (N)x is substantially complementary to the sequence of (N′)y; and the sequence of (N′)y is substantially identical to the sequence of an mRNA encoded by a target gene.

In some embodiments x=y=19. In other embodiments x=y=23. In some preferred embodiments (N)x comprises modified and unmodified ribonucleotides, and at least one unconventional moiety.

In some embodiments in (N)x the N at the 3′ terminus is a modified ribonucleotide and (N)x comprises at least 8 modified ribonucleotides. In other embodiments at least 5 of the at least 8 modified ribonucleotides are alternating beginning at the 3′ end. In some embodiments (N)x comprises an abasic moiety in one of positions 5, 6, 7, 8, 9, 10, 11, 12, 13, 14 or 15.

In some embodiments the at least one unconventional moiety in (N′)y is present at positions 15, 16, 17, or 18. In some embodiments the unconventional moiety is selected from a mirror nucleotide, an abasic ribose moiety and an abasic deoxyribose moiety. In some preferred embodiments the unconventional moiety is a mirror nucleotide, preferably an L-DNA moiety. In some embodiments an L-DNA moiety is present at position 17, position 18 or positions 17 and 18. In other embodiments the at least one unconventional moiety in (N′)y is an abasic ribose moiety or an abasic deoxyribose moiety.

In various embodiments of Structure (X) z″ is present and is selected from an abasic ribose moiety, a deoxyribose moiety; an inverted abasic ribose moiety, a deoxyribose moiety; C6-amino-Pi; a mirror nucleotide.

In yet another aspect the present invention provides a compound having Structure (K) set forth below:

| | |
|---|---|
| (K) | 5′ (N)ₓ-Z 3′ (antisense strand) |
| | 3′ Z′-(N′)_{y}-z″ 5′ (sense strand) |

wherein each of N and N′ is a ribonucleotide which may be unmodified or modified, or an unconventional moiety;
wherein each of (N)x and (N′)y is an oligonucleotide in which each consecutive N or N′ is joined to the next N or N′ by a covalent bond;
wherein Z and Z′ may be present or absent, but if present is independently 1-5 consecutive nucleotides covalently attached at the 3′ terminus of the strand in which it is present;
wherein z″ may be present or absent but if present is a capping moiety covalently attached at the 5′ terminus of (N′)y;
wherein each of x and y are independently 18 to 27;
wherein (N)x comprises a combination of modified or unmodified ribonucleotides and unconventional moieties, any modified ribonucleotide having a 2′-O-methyl on its sugar;
wherein (N′)y comprises modified or unmodified ribonucleotides and optionally an unconventional moiety, any modified ribonucleotide having a 2′OMe on its sugar;
wherein the sequence of (N)x is substantially complementary to the sequence of (N′)y; and the sequence of (N′)y is substantially identical to the sequence of an mRNA encoded by a target gene; and wherein there are less than 15 consecutive nucleotides complementary to the mRNA.

In some embodiments x=y=19. In other embodiments x=y=23. In some preferred embodiments the at least one preferred one unconventional moiety is present in (N)x and is an abasic ribose moiety or an abasic deoxyribose moiety. In other embodiments the at least one unconventional moiety is present in (N)x and is a non-base pairing nucleotide analog. In various embodiments (N′)y comprises unmodified ribonucleotides. In some embodiments (N)x comprises at least five abasic ribose moieties or abasic deoxyribose moieties or a combination thereof. In certain embodiments (N)x and/or (N′)y comprise modified ribonucleotides which do not base pair with corresponding modified or unmodified ribonucleotides in (N′)y and/or (N)x.

In various embodiments the present invention provides an siRNA set forth in Structure (L):

| | |
|---|---|
| (L) | 5′ (N)ₓ-Z 3′ (antisense strand) |
| | 3′ Z'-(N′)_{y} 5′ (sense strand) |

wherein each of N and N′ is a nucleotide selected from an unmodified ribonucleotide, a modified ribonucleotide, an unmodified deoxyribonucleotide and a modified deoxyribonucleotide;
wherein each of (N)ₓ and (N′)_{y} is an oligonucleotide in which each consecutive N or N′ is joined to the next N or N′ by a covalent bond;
wherein Z and Z′ are absent;
wherein x=y=19;
wherein in (N′)y the nucleotide in at least one of positions 15, 16, 17, 18 and 19 comprises a nucleotide selected from an abasic pseudo-nucleotide, a mirror nucleotide, a deoxyribonucleotide and a nucleotide joined to an adjacent nucleotide by a 2′-5′ internucleotide bond;
wherein (N)x comprises alternating modified ribonucleotides and unmodified ribonucleotides each modified ribonucleotide being modified so as to have a 2′-O-methyl on its sugar and the ribonucleotide located at the middle position of (N)x being modified or unmodified, preferably unmodified; and
wherein the sequence of (N)x is substantially complementary to the sequence of (N′)y; and the sequence of (N′)y is substantially identical to the mRNA of a target gene.

In some embodiments of Structure (L), in (N′)y the nucleotide in one or both of positions 17 and 18 comprises a modified nucleotide selected from an abasic pseudo-nucleotide, a mirror nucleotide and a nucleotide joined to an adjacent nucleotide by a 2′-5′ internucleotide bond. In some embodiments the mirror nucleotide is selected from L-DNA and L-RNA. In various embodiments the mirror nucleotide is L-DNA.

In various embodiments (N′)y comprises a modified nucleotide at position 15 wherein the modified nucleotide is selected from a mirror nucleotide and a deoxyribonucleotide.

In certain embodiments (N′)y further comprises a modified nucleotide or pseudo nucleotide at position 2 wherein the pseudo nucleotide may be an abasic pseudo-nucleotide analog and the modified nucleotide is optionally a mirror nucleotide.

In various embodiments the antisense strand (N)x comprises 2′O-Me modified ribonucleotides at the odd numbered positions (5′ to 3′; positions 1, 3, 5, 7, 9, 11, 13, 15, 17, 19). In some embodiments (N)x further comprises 2′O-Me modified ribonucleotides at one or both positions 2 and 18. In other embodiments (N)x comprises 2′O Me modified ribonucleotides at positions 2, 4, 6, 8, 11, 13, 15, 17, 19.

Other embodiments of the Structures above are envisaged wherein x=y=21 or wherein x=y=23; in these embodiments the modifications for (N′)y discussed above instead of being in positions 17 and 18 are in positions 19 and 20 for 21-mer oligonucleotide and 21 and 22 for 23 mer oligonucleotide; similarly the modifications in positions 15, 16, 17, 18 or 19 are in positions 17, 18, 19, 20 or 21 for the 21-mer oligonucleotide and positions 19, 20, 21, 22, or 23 for the 23-mer oligonucleotide. The 2′O Me modifications on the antisense strand are similarly adjusted. In some embodiments (N)x comprises 2′O Me modified ribonucleotides at the odd numbered positions (5′ to 3′; positions 1, 3, 5, 7, 9, 12, 14, 16, 18, 20 for the 21 mer oligonucleotide [nucleotide at position 11 unmodified] and 1, 3, 5, 7, 9, 11, 13, 15, 17, 19, 21, 23 for the 23 mer oligonucleotide [nucleotide at position 12 unmodified]. In other embodiments (N)x comprises 2′O Me modified ribonucleotides at positions 2, 4, 6, 8, 10, 12, 14, 16, 18, 20 [nucleotide at position 11 unmodified for the 21 mer oligonucleotide and at positions 2, 4, 6, 8, 10, 13, 15, 17, 19, 21, 23 for the 23 mer oligonucleotide [nucleotide at position 12 unmodified]. In some embodiments (N′)y further comprises a 5′ terminal cap nucleotide. In various embodiments the terminal cap moiety is selected from an abasic pseudo-nucleotide analog, an inverted abasic pseudo-nucleotide analog, an L-DNA nucleotide, and a C6-imine phosphate.

In other embodiments the present invention provides a compound having Structure (M) set forth below:

5′ (N)ₓ-Z 3′ (antisense strand)
3′ Z′-(N′)_{y} 5′ (sense strand)

wherein each of N and N′ is selected from a pseudo-nucleotide and a nucleotide;
wherein each nucleotide is selected from an unmodified ribonucleotide, a modified ribonucleotide, an unmodified deoxyribonucleotide and a modified deoxyribonucleotide;
wherein each of (N)ₓ and (N′)_{y} is an oligonucleotide in which each consecutive N or N′ is joined to the next N or N′ by a covalent bond;
wherein Z and Z′ are absent;
wherein each of x and y are independently 18 to 27;
wherein the sequence of (N)x is substantially complementary to the sequence of (N′)y; and the sequence of (N′)y is substantially identical to the mRNA of a target gene;
wherein at least one of N is selected from an abasic pseudo nucleotide, a non-pairing nucleotide analog and a nucleotide mismatch to the mRNA of a target gene in a position of (N)x such that (N)x comprises less than 15 consecutive nucleotides complementary to the mRNA of a target gene.

In other embodiments the present invention provides a double stranded compound having Structure (N) set forth below:

| | |
|---|---|
| (N) | 5′ (N)ₓ-Z 3′ (antisense strand) |
| | 3′ Z′-(N′)_{y} 5′ (sense strand) |

wherein each of N and N′ is a nucleotide selected from an unmodified ribonucleotide, a modified ribonucleotide, an unmodified deoxyribonucleotide and a modified deoxyribonucleotide;
wherein each of (N)ₓ and (N′)_{y} is an oligonucleotide in which each consecutive N or N′ is joined to the next N or N′ by a covalent bond;
wherein Z and Z′ are absent;
wherein each of x and y is an integer between 18 and 40;
wherein the sequence of (N)x is substantially complementary to the sequence of (N′)y; and the sequence of (N′)y is substantially identical to the mRNA of a target gene;
wherein (N)x, (N′)y or (N)x and (N′)y comprise non base-pairing modified nucleotides such that (N)x and (N′)y form less than 15 base pairs in the double stranded compound.

In other embodiments the present invention provides a compound having Structure (O) set forth below:

| | |
|---|---|
| (O) | 5′ (N)ₓ-Z 3′ (antisense strand) |
| | 3′ Z′-(N′)_{y} 5′ (sense strand) |

wherein each of N is a nucleotide selected from an unmodified ribonucleotide, a modified ribonucleotide, an unmodified deoxyribonucleotide and a modified deoxyribonucleotide;
wherein each of N′ is a nucleotide analog selected from a six membered sugar nucleotide, seven membered sugar nucleotide, morpholino moiety, peptide nucleic acid and combinations thereof;
wherein each of (N)ₓ and (N′)_{y} is an oligonucleotide in which each consecutive N or N′ is joined to the next N or N′ by a covalent bond;
wherein Z and Z′ are absent;
wherein each of x and y is an integer between 18 and 40;
wherein the sequence of (N)x is substantially complementary to the sequence of (N′)y; and the sequence of (N′)y is substantially identical to the mRNA of a target gene.

In other embodiments the present invention provides a compound having Structure (P) set forth below:

| | |
|---|---|
| (P) | 5′ (N)ₓ-Z 3′ (antisense strand) |
| | 3′ Z′-(N′)_{y} 5′ (sense strand) |

wherein each of N and N′ is a nucleotide selected from an unmodified ribonucleotide, a modified ribonucleotide, an unmodified deoxyribonucleotide and a modified deoxyribonucleotide;
wherein each of (N)ₓ and (N′)_{y} is an oligonucleotide in which each consecutive N or N′ is joined to the next N or N′ by a covalent bond;
wherein Z and Z′ are absent;
wherein each of x and y is an integer between 18 and 40;
wherein one of N or N′ in an internal position of (N)x or (N′)y or one or more of N or N′ at a terminal position of (N)x or (N′)y comprises an abasic moiety or a 2′ modified nucleotide;
wherein the sequence of (N)x is substantially complementary to the sequence of (N′)y; and the sequence of (N′)y is substantially identical to the mRNA of a target gene.

In various embodiments (N′)y comprises a modified nucleotide at position 15 wherein the modified nucleotide is selected from a mirror nucleotide and a deoxyribonucleotide.

In certain embodiments (N′)y further comprises a modified nucleotide at position 2 wherein the modified nucleotide is selected from a mirror nucleotide and an abasic pseudo-nucleotide analog.

In various embodiments the antisense strand (N)x comprises 2′O-Me modified ribonucleotides at the odd numbered positions (5′ to 3′; positions 1, 3, 5, 7, 9, 11, 13, 15, 17, 19). In some embodiments (N)ₓ further comprises 2′O-Me modified ribonucleotides at one or both positions 2 and 18. In other embodiments (N)x comprises 2′O Me modified ribonucleotides at positions 2, 4, 6, 8, 11, 13, 15, 17, 19.

The Structural motifs described above are useful with any oligonucleotide pair (sense and antisense strands) to a mammalian or non-mammalian gene. In some embodiments the mammalian gene is a human gene preferably selected from the genes for which the mRNA is provided in Tables A1-A2 (SEQ ID NOS:1-89).

In another aspect the present invention provides a pharmaceutical composition comprising a modified or unmodified compound of the present invention, in an amount effective to inhibit human gene expression wherein the compound comprises an antisense sequence, (N)ₓ; and a pharmaceutically acceptable carrier.

In yet another aspect the present invention provides a pharmaceutical composition comprising one or more modified compounds of the present invention, in an amount effective to inhibit human gene expression wherein the compound comprises an antisense sequence, (N)ₓ; and a pharmaceutically acceptable carrier.

### Pharmaceutical Compositions

While it may be possible for the compounds of the present invention to be administered as the raw chemical, it is preferable to present them as a pharmaceutical composition. Accordingly the present invention provides a pharmaceutical composition comprising one or more of the compounds of the invention; and a pharmaceutically acceptable carrier. This composition may comprise a mixture of two or more different oligonucleotides/siRNAs.

The invention further provides a pharmaceutical composition comprising at least one compound of the invention covalently or non-covalently bound to one or more compounds of the invention in an amount effective to inhibit one or more genes as disclosed above; and a pharmaceutically acceptable carrier. The compound may be processed intracellularly by endogenous cellular complexes to produce one or more oligoribonucleotides of the invention.

The invention further provides a pharmaceutical composition comprising a pharmaceutically acceptable carrier and one or more of the compounds of the invention in an amount effective to down-regulate expression in a cell of a human gene, the compound comprising a sequence substantially complementary to the sequence of (N)ₓ.

The present invention also provides for a process of preparing a pharmaceutical composition, which comprises:

providing one or more compounds of the invention; and
admixing said compound with a pharmaceutically acceptable carrier.

The present invention also provides for a process of preparing a pharmaceutical composition, which comprises admixing one or more compounds of the present invention with a pharmaceutically acceptable carrier.

In a preferred embodiment, the compound used in the preparation of a pharmaceutical composition is admixed with a carrier in a pharmaceutically effective dose. In a particular embodiment the compound of the present invention is conjugated to a steroid or to a lipid or to another suitable molecule e.g. to cholesterol.

Additionally, the invention provides a method of inhibiting the expression of a gene of the present invention by at least 50% as compared to a control comprising contacting an mRNA transcript of the gene of the present invention with one or more of the compounds of the invention. In some embodiments an active siRNA compound inhibits gene expression at a level of at least 50%, 60% or 70% as compared to control. In certain preferred embodiments inhibition is at a level of at least 75%, 80% or 90% as compared to control.

In one embodiment the oligoribonucleotide is inhibiting one or more of the genes of the present invention, whereby the inhibition is selected from the group comprising inhibition of gene function, inhibition of polypeptide and inhibition of mRNA expression.

In one embodiment the compound inhibits a polypeptide, whereby the inhibition is selected from the group comprising inhibition of function (which may be examined by an enzymatic assay or a binding assay with a known interactor of the native gene/polypeptide, inter alia), inhibition of protein (which may be examined by Western blotting, ELISA or immuno-precipitation, inter alia) and inhibition of mRNA expression (which may be examined by Northern blotting, quantitative RT-PCR, in-situ hybridisation or microarray hybridisation, inter alia).

### Delivery

The siRNA molecules of the present invention may be delivered to the target tissue by direct application of the naked molecules prepared with a carrier or a diluent.

The term “naked siRNA” refers to siRNA molecules that are free from any delivery vehicle or formulation that acts to assist, promote or facilitate entry into the cell, including viral sequences, viral particles, liposome formulations, lipofectin or precipitating agents and the like. For example, siRNA in PBS is “naked siRNA”. In preferred embodiments of the invention the siRNA is delivered as naked siRNA.

siRNA molecules may be delivered in liposome or lipofectin formulations and the like and can be prepared by methods well known to those skilled in the art. Such methods are described, for example, in U.S. Pat. Nos. 5,593,972, 5,589,466, and 5,580,859, which are herein incorporated by reference.

Delivery systems aimed specifically at the enhanced and improved delivery of siRNA into mammalian cells have been developed, (see, for example, Shen et al FEBS Let. 2003, 539:111-114; Xia et al., Nat. Biotech. 2002, 20:1006-1010; Reich et al., Mol. Vision 2003, 9: 210-216; Sorensen et al., J. Mol. Biol. 2003. 327: 761-766; Lewis et al., Nat. Gen. 2002, 32: 107-108 and Simeoni et al., NAR 2003, 31, 11: 2717-2724).

The pharmaceutically acceptable carriers, solvents, diluents, excipients, adjuvants and vehicles as well as implant carriers generally refer to inert, non-toxic solid or liquid fillers, diluents or encapsulating material not reacting with the active ingredients of the invention and they include liposomes and microspheres. Examples of delivery systems useful in the present invention include U.S. Pat. Nos. 5,225,182; 5,169,383; 5,167,616; 4,959,217; 4,925,678; 4,487,603; 4,486,194; 4,447,233; 4,447,224; 4,439,196; and 4,475,196. Many other such implants, delivery systems, and modules are well known to those skilled in the art.

Any suitable route of administration may be employed for providing the subject with an effective dosage. For example, oral, rectal, parenteral (subcutaneous, intramuscular, intravenous), transdermal, and like forms of administration may be employed. Dosage forms may include tablets, troches, dispersions, suspensions, solutions, capsules, patches, and the like. In preferred embodiments of the present invention the siRNA reaches its target cell systemically, via the circulatory system. The siRNAs or pharmaceutical compositions of the present invention are administered and dosed in accordance with good medical practice, taking into account the clinical condition of the individual patient, the disease to be treated, the site and method of administration, scheduling of administration, patient age, sex, body weight and other factors known to medical practitioners.

The “therapeutically effective dose” for purposes herein is thus determined by such considerations as are known in the art. The dose must be effective to achieve improvement including but not limited to improved survival rate or more rapid recovery, or improvement or elimination of symptoms and other indicators as are selected as appropriate measures by those skilled in the art.

In general, the active dose of compound for humans is in the range of from 1 ng/kg to about 20-100 mg/kg body weight per day, preferably about 0.01 mg to about 2-10 mg/kg body weight per day, in a regimen of one dose per day or twice or three or more times per day for a period of 1-4 weeks or longer.

The compounds of the present invention can be administered by any of the conventional routes of administration. It should be noted that the compound can be administered as the compound or as pharmaceutically acceptable salt and can be administered alone or as an active ingredient in combination with pharmaceutically acceptable carriers, solvents, diluents, excipients, adjuvants and vehicles.

According to the present invention the preferred method of delivery is systemic delivery.

The compounds can be administered orally, subcutaneously or parenterally including intravenous, intraarterial, intramuscular, intraperitoneally, and intranasal, inhalation, transtymopanic administration as well as intrathecal and infusion techniques. Implants of the compounds are also useful. Liquid forms may be prepared for injection, the term including subcutaneous, transdermal, intravenous, intramuscular, intrathecal, and other parental routes of administration. The liquid compositions include aqueous solutions, with and without organic co-solvents, aqueous or oil suspensions, emulsions with edible oils, as well as similar pharmaceutical vehicles. In a particular embodiment, the administration comprises intravenous administration. In another embodiment the administration comprises topical or local administration.

The phrases “systemic delivery”, “systemic administration”, “administered systematically” refer to the administration of a compound, or composition such that it enters the patient's circulatory system.

These compounds may be administered to humans and other animals for therapy by any suitable route of administration, including orally, nasally, as by, for example, a spray, rectally, intravaginally, parenterally, intracisternally and topically, as by powders, ointments or drops, including buccally and sublingually.

In addition, in certain embodiments the compositions for use in the novel treatments of the present invention may be formed as aerosols, for example for intranasal administration.

In certain embodiments, oral compositions (such as tablets, suspensions, solutions) may be effective for local delivery to the oral cavity such as oral composition suitable for mouthwash for the treatment of oral mucositis.

In preferred embodiments the subject being treated is a warm-blooded animal and, in particular, mammals including human.

### Methods of Treatment

In one aspect, the present invention relates to a method for the treatment of a subject in need of treatment for a disease or disorder associated with expression of a gene listed in Tables A1 and A2 in an immature myeloid cell of the subject, comprising administering to the subject an amount of an inhibitor which inhibits expression of at least one of the genes. In some embodiments more than one siRNA compound to one or more than one gene target is administered.

In preferred embodiments the subject being treated is a warm-blooded animal and, in particular, mammals including human.

The methods of the invention comprise administering to the subject one or more inhibitory compounds which down-regulate expression of the genes of Tables A1-A2; and in particular siRNA in a therapeutically effective dose so as to thereby treat the subject. In certain preferred embodiments an siRNA compound comprises an antisense and sense sequence pair set forth in Tables B or G. Certain preferred siRNA compounds are listed in Table G and set forth in SEQ ID NOS:24,076-24,117)

The term “treatment” refers to both therapeutic treatment and prophylactic or preventative measures, wherein the object is to prevent or slow down, attenuate the related disorder as listed above. Those in need of treatment include those already experiencing the disease or condition, those prone to having the disease or condition, and those in which the disease or condition is to be prevented. The compounds of the invention may be administered before, during or subsequent to the onset of the disease or condition or symptoms associated therewith. In cases where treatment is for the purpose of prevention, then the present invention relates to a method for delaying the onset of or averting the development of the disease or disorder.

In one aspect the present invention also provides a method of treating cancer in a subject in need thereof which comprises administering systemically to the subject a therapeutically effective amount of an oligonucleotide which inhibits expression of a gene expressed in a myeloid cell in the subject in an amount effective to treat the cancer.

In another aspect the present invention provides a method of treating a subject suffering from a disorder in which the level of T cell receptor zeta chain (CD3ζ; CD3 zeta) is reduced or absent comprising administering to the subject an oligonucleotide which inhibits expression of a NOX gene expressed in a myeloid cell in the subject in an amount effective to treat the disorder.

In a further aspect the present invention provides a method of reducing tumor vascularization and tumor progression in a subject in need thereof which comprises administering systemically to the subject a therapeutically effective amount of an oligonucleotide which inhibits expression of a gene expressed in a myeloid cell in the subject in an amount effective to reduce tumor vascularization and tumor progression.

By “cancer” is meant any disease that is caused by or results in inappropriately high levels of cell division, inappropriately low levels of apoptosis, or both. Examples of cancer include, without limitation, leukemias (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic rnyelocytic leukemia, chronic lymphocytic leukemia), polycythemia vera, lymphoma (Hodgkin's disease, non-Hodgkin's disease), Waldenstrom's macroglobulinemia, heavy chain disease, and solid tumors such as sarcomas and carcinomas (e.g., fibrosarcoma, myxosarcoma, liposarcoma, chondrosarcoma, osteogenic sarcoma, chordoma, angiosarcoma, endotheliosarcoma, lymphangio sarcoma, lymphangioendotheliosarcoma, synovioma, mesothelioma, Ewing's tumor, leiomyo sarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, nile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, astrocytoma, medulloblastoma, crailiopharyngioma, ependymoma, pinealoma, hemangioblastoma, acoustic neuroma, oligodenroglioma, schwamioma, meningioma, melanoma, neuroblastoma, and retinoblastoma).

### Combination Therapy

One aspect of the present invention relates to combination therapy. In one embodiment, the co-administration of two or more therapeutic agents achieves a synergistic effect, i.e., a therapeutic affect that is greater than the sum of the therapeutic effects of the individual components of the combination. In another embodiment, the co-administration of two or more therapeutic agents achieves an additive effect.

The active ingredients that comprise a combination therapy may be administered together via a single dosage form or by separate administration of each active agent. In certain embodiments, the first and second therapeutic agents are administered in a single dosage form. The agents may be formulated into a single tablet, pill, capsule, or solution for parenteral administration and the like. Alternatively, the first therapeutic agent and the second therapeutic agents may be administered as separate compositions. The first active agent may be administered at the same time as the second active agent or the first active agent may be administered intermittently with the second active agent. The length of time between administration of the first and second therapeutic agent may be adjusted to achieve the desired therapeutic effect. For example, the second therapeutic agent may be administered only a few minutes (e.g., 1, 2, 5, 10, 30, or 60 min) or several hours (e.g., 2, 4, 6, 10, 12, 24, or 36 hr) after administration of the first therapeutic agent. In certain embodiments, it may be advantageous to administer more than one dosage of one of the therapeutic agents between administrations of the second therapeutic agent. For example, the second therapeutic agent may be administered at 2 hours and then again at 10 hours following administration of the first therapeutic agent. Alternatively, it may be advantageous to administer more than one dosage of the first therapeutic agent between administrations of the second therapeutic agent. Importantly, it is preferred that the therapeutic effects of each active ingredient overlap for at least a portion of the duration of each therapeutic agent so that the overall therapeutic effect of the combination therapy is attributable in part to the combined or synergistic effects of the combination therapy.

The present invention relates to the use of compounds which down-regulate the expression of the genes of the invention particularly to novel small interfering RNAs (siRNAs), in the treatment of the following diseases or conditions in which inhibition of the expression of the Methods, molecules and compositions which inhibit the genes of the invention are discussed herein at length, and any of said molecules and/or compositions may be beneficially employed in the treatment of a subject suffering from any of said conditions.

The compounds of the present invention can be administered alone or in combination with a chemotherapeutic agent. A “chemotherapeutic agent” is a chemical compound useful in the treatment of cancer. Examples of chemotherapeutic agents include alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); delta-9-tetrahydrocannabinol (dronabinol, MARINOL®); beta-lapachone; lapachol; colchicines; betulinic acid; a camptothecin (including the synthetic analogue topotecan (HYCAMTIN®), CPT-11 (irinotecan, CAMPTOSAR®), acetylcamptothecin, scopolectin, and 9-aminocamptothecin); bryostatin; callystatin; CC-1065 (including its adozelesin, carzelesin and bizelesin synthetic analogues); podophyllotoxin; podophyllinic acid; teniposide; cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gamma 1I and calicheamicin omegaI1 (see, e.g., Agnew, Chem. Intl. Ed. Engl., 1994. 33: 183-186); dynemicin, including dynemicin A; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, doxorubicin (including ADRIAMYCIN®, morpholino-doxorubicin, cyanomorpholino-doxorubicin, 2-pyrrolino-doxorubicin, doxorubicin HCl liposome injection (DOXIL®) and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate, gemcitabine (GEMZAR®), tegafur (UFTORAL®), capecitabine (XELODA®), an epothilone, and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, cannofiir, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; 2-ethylhydrazide; procarbazine; PSK® polysaccharide complex; razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine (ELDISINE®, FILDESIN®); dacarbazine; mannomustine; mitobronitol; mitolactol; pipobroman; gacytosine; arabinoside (“Ara-C”); thiotepa; taxoids, e.g., paclitaxel (TAXOL®), albumin-engineered nanoparticle formulation of paclitaxel (ABRAXANE™), and doxetaxel (TAXOTERE®); chloranbucil; 6-thioguanine; mercaptopurine; methotrexate; a platinum analog such as cisplatin and carboplatin; vinblastine (VELBAN®); platinum; etoposide (VP-16); ifosfamide; mitoxantrone; vincristine (ONCOVIN®); oxaliplatin; leucovovin; vinorelbine (NAVELBINE®); novantrone; edatrexate; daunomycin; aminopterin; ibandronate; topoisomerase inhibitor RFS 2000; difluoromethylornithine (DMFO); a retinoid such as retinoic acid; pharmaceutically acceptable salts, acids or derivatives of any of the above; as well as combinations of two or more of the above such as CHOP, an abbreviation for a combined therapy of cyclophosphamide, doxorubicin, vincristine, and prednisolone, and FOLFOX, an abbreviation for a treatment regimen with oxaliplatin (ELOXATIN®) combined with 5-FU and leucovovin.

Also included in this definition are anti-hormonal agents that act to regulate, reduce, block, or inhibit the effects of hormones that can promote the growth of cancer, and are often in the form of systemic, or whole-body treatment. They may be hormones themselves. Examples include anti-estrogens and selective estrogen receptor modulators (SERMs), including, for example, tamoxifen (including NOLVADEX® tamoxifen), raloxifene (EVISTA®), droloxifene, 4-hydroxytamoxifen, trioxifene, keoxifene, LY117018, onapristone, and toremifene (FARESTON®); anti-progesterones; estrogen receptor down-regulators (ERDs); agents that function to suppress or shut down the ovaries, for example, leutinizing hormone-releasing hormone (LHRH) agonists such as leuprolide acetate (LUPRON® and ELIGARD®), goserelin acetate, buserelin acetate and tripterelin; other anti-androgens such as flutamide, nilutamide and bicalutamide; and aromatase inhibitors such as, for example, 4(5)-imidazoles, aminoglutethimide, megestrol acetate (MEGASE®), exemestane (AROMASIN®), formestanie, fadrozole, vorozole (RIVISOR®), letrozole (FEMARA®), and anastrozole (ARIMIDEX®). In addition, bisphosphonates such as clodronate (for example, BONEFOS® or OSTAC®), etidronate (DIDROCAL®), NE-58095, zoledronic acid/zoledronate (ZOMETA®), alendronate (FOSAMAX®), pamidronate (AREDIA®), tiludronate (SKELID®), or risedronate (ACTONEL®); as well as troxacitabine (a 1,3-dioxolane nucleoside cytosine analog); siRNA, ribozyme and antisense oligonucleotides, particularly those that inhibit expression of genes in signaling pathways implicated in aberrant cell proliferation; vaccines such as THERATOPE® vaccine and gene therapy vaccines, for example, ALLOVECTIN® vaccine, LEUVECTIN® vaccine, and VAXID® vaccine; topoisomerase 1 inhibitor (e.g., LURTOTECAN®); rmRH (e.g., ABARELIX®); lapatinib ditosylate (an ErbB-2 and EGFR dual tyrosine kinase small-molecule inhibitor also known as GW572016); COX-2 inhibitors such as celecoxib (CELEBREX®; 4-(5-(4-methylphenyl)-3-(trifluoromethyl)-1H-pyrazol-1-yl) benzenesulfonamide; and pharmaceutically acceptable salts, acids or derivatives of any of the above.

siRNA Synthesis

The compounds of the present invention can be synthesized by any of the methods that are well-known in the art for synthesis of ribonucleic (or deoxyribonucleic) oligonucleotides. Such synthesis is, among others, described in Beaucage and Iyer, Tetrahedron 1992; 48:2223-2311; Beaucage and Iyer, Tetrahedron 1993; 49: 6123-6194 and Caruthers, et. al., Methods Enzymol. 1987; 154: 287-313; the synthesis of thioates is, among others, described in Eckstein, Annu. Rev. Biochem. 1985; 54: 367-402, the synthesis of RNA molecules is described in Sproat, in Humana Press 2005 edited by Herdewijn P.; Kap. 2: 17-31 and respective downstream processes are, among others, described in Pingoud et. al., in IRL Press 1989 edited by Oliver; Kap. 7: 183-208.

Other synthetic procedures are known in the art e.g. the procedures as described in Usman et al., J. Am. Chem. Soc., 1987, 109:7845; Scaringe et al., NAR, 1990, 18:5433; Wincott et al., NAR 1995, 23:2677-2684; and Wincott et al., Methods Mol. Bio., 1997, 74:59, and these procedures may make use of common nucleic acid protecting and coupling groups, such as dimethoxytrityl at the 5′-end, and phosphoramidites at the 3′-end. The modified (e.g. 2′-O-methylated) nucleotides and unmodified nucleotides are incorporated as desired.

The oligonucleotides of the present invention can be synthesized separately and joined together post-synthetically, for example, by ligation (Moore et al., Science 1992, 256:9923; International Patent Publication No. WO 93/23569; Shabarova et al., NAR 1991, 19:4247; Bellon et al., Nucleosides & Nucleotides, 1997, 16:951; Bellon et al., Bioconjugate Chem 1997, 8:204), or by hybridization following synthesis and/or deprotection.

It is noted that a commercially available machine (available, inter alia, from Applied Biosystems) can be used; the oligonucleotides are prepared according to the sequences disclosed herein. Overlapping pairs of chemically synthesized fragments can be ligated using methods well known in the art (e.g., see U.S. Pat. No. 6,121,426). The strands are synthesized separately and then are annealed to each other in the tube. Then, the double-stranded siRNAs are separated from the single-stranded oligonucleotides that were not annealed (e.g. because of the excess of one of them) by HPLC. In relation to the siRNAs or siRNA fragments of the present invention, two or more such sequences can be synthesized and linked together for use in the present invention.

The compounds of the invention can also be synthesized via tandem synthesis methodology, as described for example in US Patent Publication No. 2004/0019001 (McSwiggen), and in PCT Patent Publication No. WO 2007/091269 (assigned to the assignee of the present invention and incorporated herein in its entirety by reference) wherein both siRNA strands are synthesized as a single contiguous oligonucleotide fragment or strand separated by a cleavable linker which is subsequently cleaved to provide separate siRNA fragments or strands that hybridize and permit purification of the siRNA duplex. The linker can be a polynucleotide linker or a non-nucleotide linker.

The present invention further provides for a pharmaceutical composition comprising two or more siRNA molecules for the treatment of any of the diseases and conditions mentioned herein, whereby said two molecules may be physically mixed together in the pharmaceutical composition in amounts which generate equal or otherwise beneficial activity, or may be covalently or non-covalently bound, or joined together by a nucleic acid linker of a length ranging from 2-100, preferably 2-50 or 2-10 nucleotides.

Thus, the siRNA molecules may be covalently or non-covalently bound or joined by a linker to form a tandem siRNA compound. Such tandem siRNA compounds comprising two siRNA sequences are typically about 38-150 nucleotides in length, more preferably 38 or 40-60 nucleotides in length, and longer accordingly if more than two siRNA sequences are included in the tandem molecule. A longer tandem compound comprised of two or more longer sequences which encode siRNA produced via internal cellular processing, e.g., long dsRNAs, is also envisaged, as is a tandem molecule encoding two or more shRNAs. Such tandem molecules are also considered to be a part of the present invention. A tandem compound comprising two or more siRNAs sequences of the invention is envisaged.

Additionally, the siRNA disclosed herein or any nucleic acid molecule comprising or encoding such siRNA can be linked or bound (covalently or non-covalently) to antibodies (including aptamer molecules) against cell surface internalizable molecules expressed on the target cells, in order to achieve enhanced targeting for treatment of the diseases disclosed herein. For example, anti-Fas antibody (preferably a neutralizing antibody) may be combined (covalently or non-covalently) with any of the siRNA compounds.

The compounds of the present invention are delivered either directly or with viral or non-viral vectors. When delivered directly the sequences are generally rendered nuclease resistant. Alternatively the sequences are incorporated into expression cassettes or constructs such that the sequence is expressed in the cell as discussed herein below. Generally the construct contains the proper regulatory sequence or promoter to allow the sequence to be expressed in the targeted cell. Vectors optionally used for delivery of the compounds of the present invention are commercially available, and may be modified for the purpose of delivery of the compounds of the present invention by methods known to one of skill in the art.

It is also envisaged that a long oligonucleotide (typically 25-500 nucleotides in length) comprising one or more stem and loop structures, where stem regions comprise the sequences of the oligonucleotides of the invention, may be delivered in a carrier, preferably a pharmaceutically acceptable carrier, and may be processed intracellularly by endogenous cellular complexes (e.g. by DROSHA and DICER as described above) to produce one or more smaller double stranded oligonucleotides (siRNAs) which are oligonucleotides of the invention. This oligonucleotide can be termed a tandem shRNA construct. It is envisaged that this long oligonucleotide is a single stranded oligonucleotide comprising one or more stem and loop structures, wherein each stem region comprises a sense and corresponding antisense siRNA sequence of the genes of the invention.

### RNA Interference

A number of PCT applications have recently been published that relate to the RNAi phenomenon. These include: PCT publication WO 00/44895; PCT publication WO 00/49035; PCT publication WO 00/63364; PCT publication WO 01/36641; PCT publication WO 01/36646; PCT publication WO 99/32619; PCT publication WO 00/44914; PCT publication WO 01/29058; and PCT publication WO 01/75164.

RNA interference (RNAi) is based on the ability of dsRNA species to enter a cytoplasmic protein complex, where it is then targeted to the complementary cellular RNA and specifically degrade it. The RNA interference response features an endonuclease complex containing an siRNA, commonly referred to as an RNA-induced silencing complex (RISC), which mediates cleavage of single-stranded RNA having a sequence complementary to the antisense strand of the siRNA duplex. Cleavage of the target RNA may take place in the middle of the region complementary to the antisense strand of the siRNA duplex (Elbashir et al., Genes Dev., 2001, 15(2):188-200). In more detail, longer dsRNAs are digested into short (17-29 bp) dsRNA fragments (also referred to as short inhibitory RNAs, “siRNAs”) by type III RNAses (DICER, DROSHA, etc.; Bernstein et al., Nature, 2001, 409(6818):363-6; Lee et al., Nature, 2003, 425(6956):415-9). The RISC protein complex recognizes these fragments and complementary mRNA. The whole process is culminated by endonuclease cleavage of target mRNA (McManus & Sharp, Nature Rev Genet, 2002, 3(10):737-47; Paddison & Hannon, Curr Opin Mol. Ther. 2003, 5(3):217-24). (For additional information on these terms and proposed mechanisms, see for example Bernstein et al., RNA 2001, 7(11):1509-21; Nishikura, Cell 2001, 107(4):415-8 and PCT publication WO 01/36646).

Several groups have described the development of DNA-based vectors capable of generating siRNA within cells. The method generally involves transcription of short hairpin RNAs that are efficiently processed to form siRNAs within cells (Paddison et al. PNAS USA 2002, 99:1443-1448; Paddison et al. Genes & Dev 2002, 16:948-958; Sui et al. PNAS USA 2002, 8:5515-5520; and Brummelkamp et al. Science 2002, 296:550-553). These reports describe methods to generate siRNAs capable of specifically targeting numerous endogenously and exogenously expressed genes.

The invention has been described in an illustrative manner, and it is to be understood that the terminology used is intended to be in the nature of words of description rather than of limitation.

Obviously, many modifications and variations of the present invention are possible in light of the above teachings. It is, therefore, to be understood that within the scope of the appended claims, the invention can be practiced otherwise than as specifically described.

Throughout this application, various publications, including United States patents, are referenced by author and year and patents by number. The disclosures of these publications and patents and patent applications in their entireties are hereby incorporated by reference into this application in order to more fully describe the state of the art to which this invention pertains.

The present invention is illustrated in detail below with reference to examples, but is not to be construed as being limited thereto.

Citation of any document herein is not intended as an admission that such document is pertinent prior art, or considered material to the patentability of any claim of the present application. Any statement as to content or a date of any document is based on the information available to applicant at the time of filing and does not constitute an admission as to the correctness of such a statement.

### EXAMPLES

Without further elaboration, it is believed that one skilled in the art can, using the preceding description, utilize the present invention to its fullest extent. The following preferred specific embodiments are, therefore, to be construed as merely illustrative, and not limitative of the claimed invention in any way.

Standard molecular biology protocols known in the art not specifically described herein are generally followed essentially as in Sambrook et al., Molecular cloning: A laboratory manual, Cold Springs Harbor Laboratory, New-York (1989, 1992), and in Ausubel et al., Current Protocols in Molecular Biology, John Wiley and Sons, Baltimore, Md. (1988), and as in Ausubel et al., Current Protocols in Molecular Biology, John Wiley and Sons, Baltimore, Md. (1989) and as in Perbal, A Practical Guide to Molecular Cloning, John Wiley & Sons, New York (1988), and as in Watson et al., Recombinant DNA, Scientific American Books, New York and in Birren et al (eds) Genome Analysis: A Laboratory Manual Series, Vols. 1-4 Cold Spring Harbor Laboratory Press, New York (1998) and methodology as set forth in U.S. Pat. Nos. 4,666,828; 4,683,202; 4,801,531; 5,192,659 and 5,272,057 and incorporated herein by reference. Polymerase chain reaction (PCR) was carried out as in standard PCR Protocols: A Guide To Methods And Applications, Academic Press, San Diego, Calif. (1990). In situ PCR in combination with Flow Cytometry (FACS) can be used for detection of cells containing specific DNA and mRNA sequences (Testoni et al., Blood 1996, 87:3822.) Methods of performing RT-PCR are well known in the art.

### Cell Culture

HeLa cells (American Type Culture Collection) were cultured as described in Czauderna, et al. (NAR, 2003. 31:670-82). Human keratinocytes were cultured at 37° C. in Dulbecco's modified Eagle medium (DMEM) containing 10% FCS. The mouse cell line, B16V (American Type Culture Collection), was cultured at 37° C. in Dulbecco's modified Eagle medium (DMEM) containing 10% FCS. Culture conditions were as described in (Methods Find Exp Clin Pharmacol. 1997, 19(4):231-9).

In each case, the cells were subject to the experiments as described herein at a density of about 50,000 cells per well and the double-stranded nucleic acid according to the present invention was added at 20 nM, whereby the double-stranded nucleic acid was complexed using 1 μg/ml of a proprietary lipid as described below.

### Induction of Hypoxia-Like Conditions

The cells were treated with CoCl₂ for inducing a hypoxia-like condition as follows: siRNA transfections were carried out in 10-cm plates (30-50% confluency) as described by Czauderna et al., (2003, NAR 31(11)2705-16) and Kretschmer et al., (2003 Oncogene. 22(43):6748-63). Briefly, siRNA were transfected by adding a preformed 10× concentrated complex of GB and lipid in serum-free medium to cells in complete medium. The total transfection volume was 10 ml. The final lipid concentration was 1.0 μg/ml; the final siRNA concentration was 20 nM unless otherwise stated. Induction of the hypoxic responses was carried out by adding CoCl₂ (100 μM) directly to the tissue culture medium 24 h before lysis.

Preparation of Cell Extracts and Immuno Blotting: the Preparation of Cell Extracts and immunoblot analysis were carried out essentially as described (Klippel et al. Mol Cell Biol, 1998. 18:5699-711; Klippel, A., et al., Mol Cell Biol, 1996. 16:4117-27).

### Example 1

### In Vitro Testing of siRNA Compounds

About 1.5−2×10⁵ tested cells (HeLa cells and/or 293T cells for siRNA targeting human genes and NRK52 cells and/or NMUMG cells for siRNA targeting the rat/mouse gene) were seeded per well in 6 wells plate (70-80% confluent).

24 hour later, cells were transfected with siRNA compounds using the Lipofectamine™ 2000 reagent (Invitrogen) at final concentrations of 5 nM or 20 nM. The cells were incubated at 37° C. in a CO₂ incubator for 72 h.

As positive control for transfection PTEN-Cy3 labeled siRNA compounds were used. An additional positive control used was a blunt-ended 19-mer siRNA, i.e. x=y=19 wherein Z and Z′ are both absent. This siRNA was non-phosphorylated and had alternating ribonucleotides modified at the 2′ position of the sugar residue in both the antisense and the sense strands, wherein the moiety at the 2′ position is methoxy (2′-O-methyl) and wherein the ribonucleotides at the 5′ and 3′ termini of the antisense strand are modified in their sugar residues, and the ribonucleotides at the 5′ and 3′ termini of the sense strand are unmodified in their sugar residues. GFP siRNA compounds were used as negative control for siRNA activity.

At 72 h after transfection, cells were harvested and RNA was extracted from cells. Transfection efficiency was tested by fluorescent microscopy.

The percent of inhibition of gene expression using specific preferred siRNA structures was determined using qPCR analysis of a target gene in cells expressing the endogenous gene.

In general, the siRNAs having specific sequences that were selected for in vitro testing were specific for human and a second species such as rat or rabbit genes. Similar results are obtained using siRNAs having these RNA sequences and modified as described herein.

### Example 2

### Distribution and Activity of siRNA in Bone Marrow

Time-Dependent Distribution of siRNA in Organs and Tissues

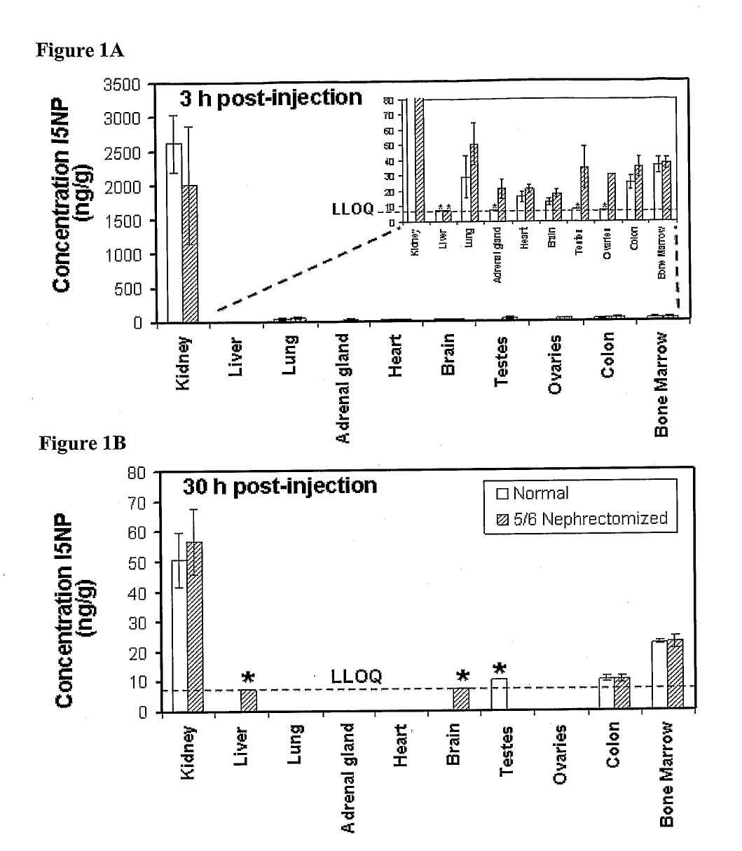

Distribution of proprietary siRNA was measured in rat tissues and organs following four consequent bolus intravenous injections of 10 mg/kg siRNA (mouse p53) given over a period of 6 hrs with 1.5-2 hrs intervals. The analysis was done in normal rats and those with abnormal kidney function since oligonucleotides are mainly excreted through kidney. The siRNA concentration in tissues was measured at 3 hrs and 30 hrs after the last siRNA administration using a quantitative method developed for this particular siRNA molecule. As shown in FIG. 1, siRNA was detected in bone marrow (BM) cells at least as early as 3 h after the last siRNA injection and was one of the highest among the organs tested, ˜40 ng/g tissue (FIG. 1A). The siRNA concentration in BM appeared relatively stable and declined only by 30% over the next 27 hours (FIG. 1B). siRNA retention in BM was similar in both normal and 5/6 nephrectomized rats.

Activity of siRNA in Bone Marrow Cells

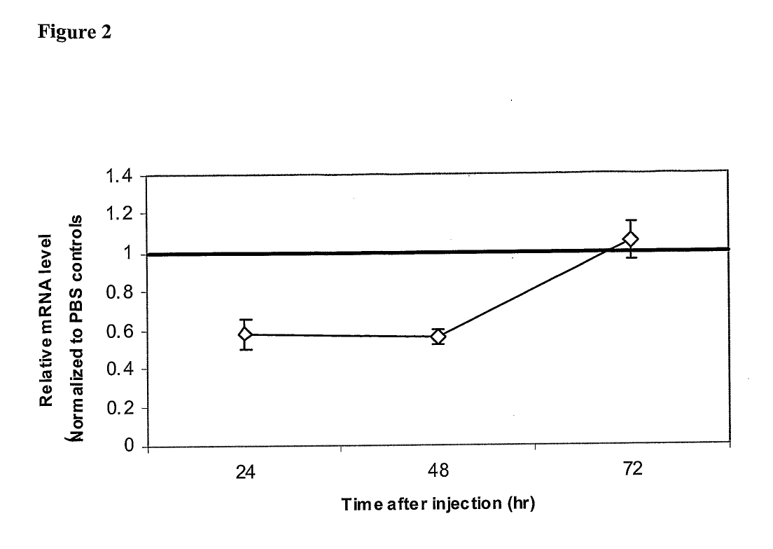

Activity of siRNA in rat BM cells (flushed out from the bone) was tested at 24, 48 and 72 hrs after its single intravenous (i.v.) bolus administration at concentration 12 mg/kg by real-time PCR analysis of the target gene expression. 40% reduction in target gene mRNA level compared to control was detected at 24-48 h after i.v. administration of siRNA (FIG. 2). mRNA levels returned to basal ones by 72 h after administration probably as a result of siRNA clearance or maturation of cells that absorbed siRNA with their subsequent escape from BM into the circulation. A 40% reduction in target gene expression may reflect the fact that only part of BM cells are targeted by siRNA. FIG. 2 shows siRNA activity in rat BM cells 24, 48 and 72 hrs following single bolus intravenous injection. The graph represents expression levels of mRNA targeted by the siRNA. Normal mRNA expression level is shown as 1 (black horizontal line).

Distribution of siRNA in Bone Marrow Cells

To determine BM mononuclear cell populations (further on, referred as BM cells) that are targeted by siRNA, Cy3-labeled siRNA (REDD14) was injected either intraperitonealy or intravenously to mice at a dose of 72 mg/kg. Distribution of siRNA molecules in the bone marrow and in peripheral blood cells was evaluated 4 h and 24 h following one administration or after two siRNA injections with 24 h intervals between injections (48 h after the first injection). In parallel, Cy3-positive cells were identified based on their unique size, granularity and specific surface markers.

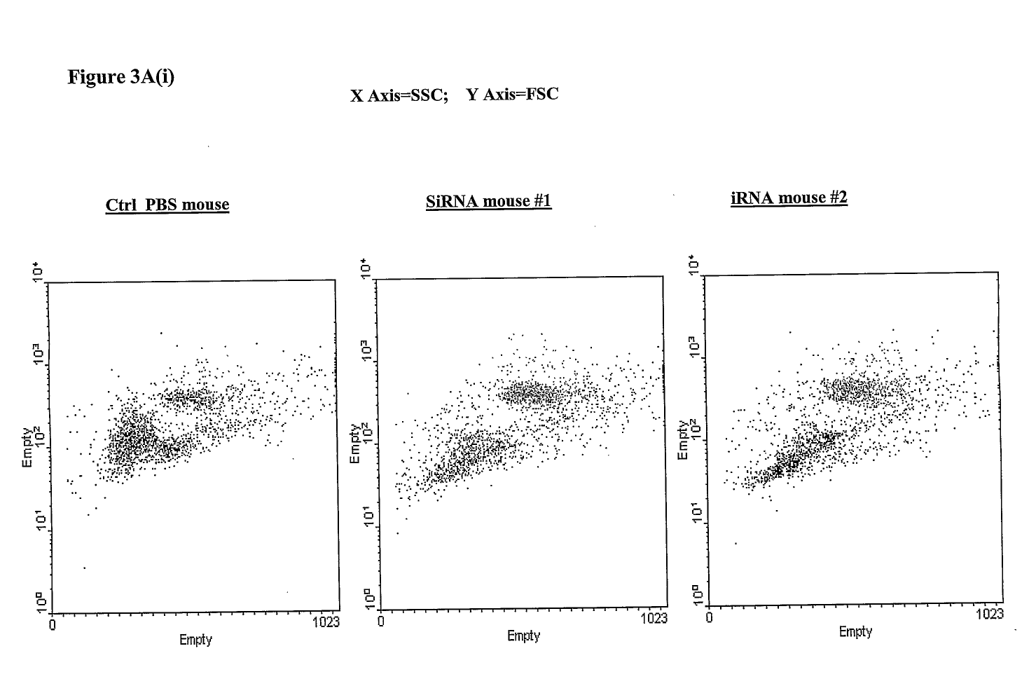

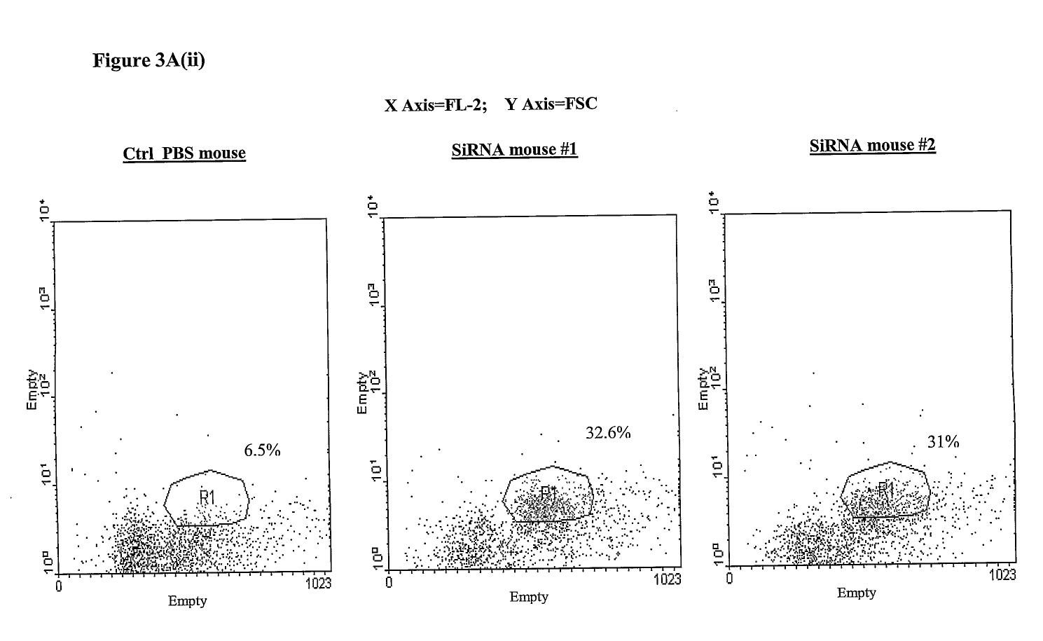

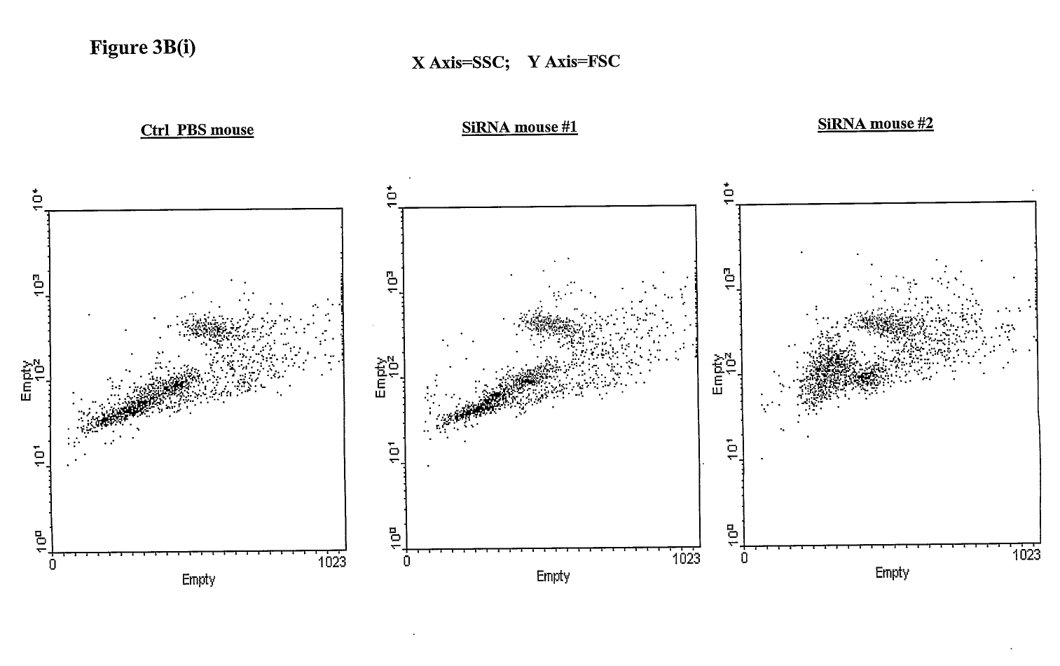

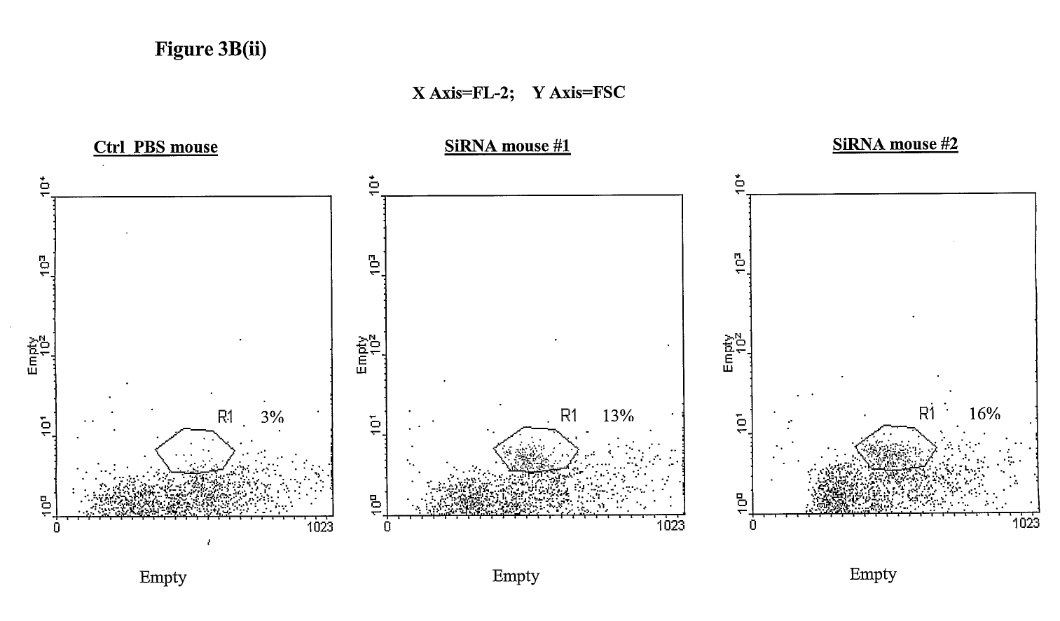

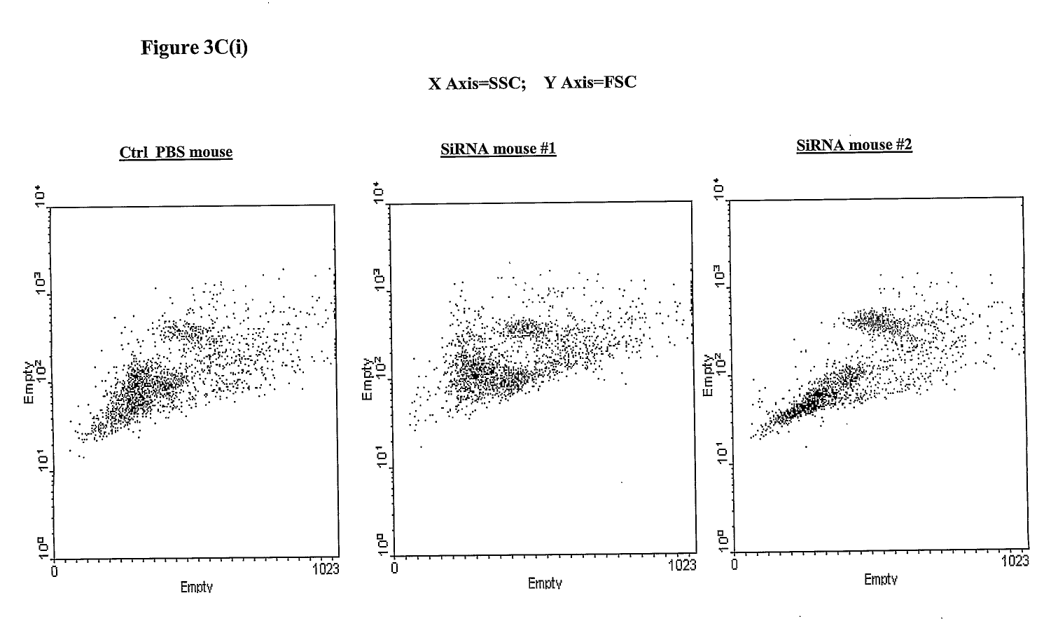

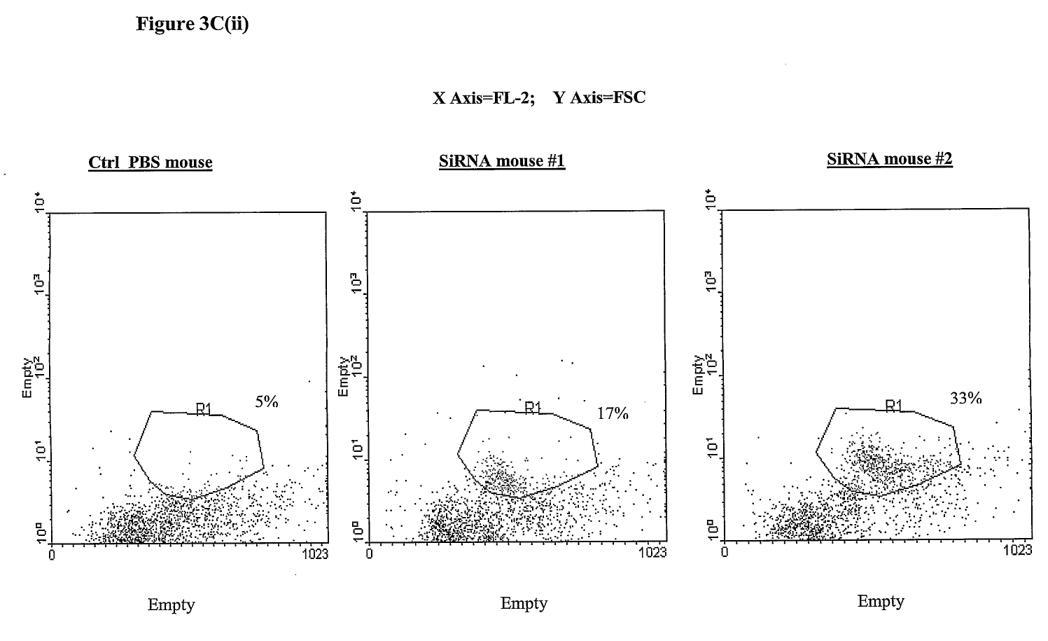

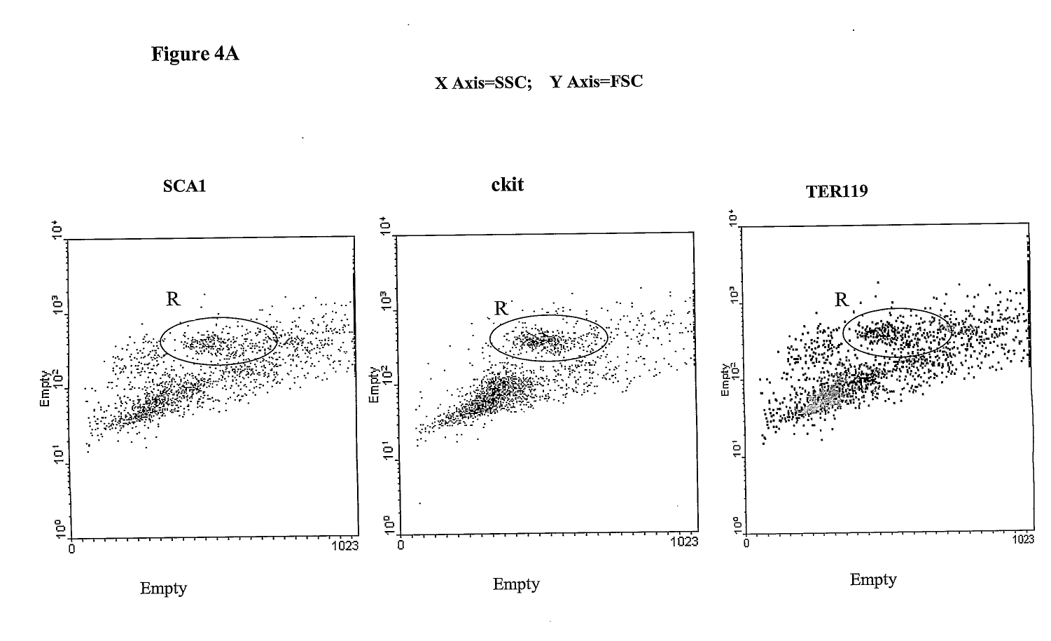

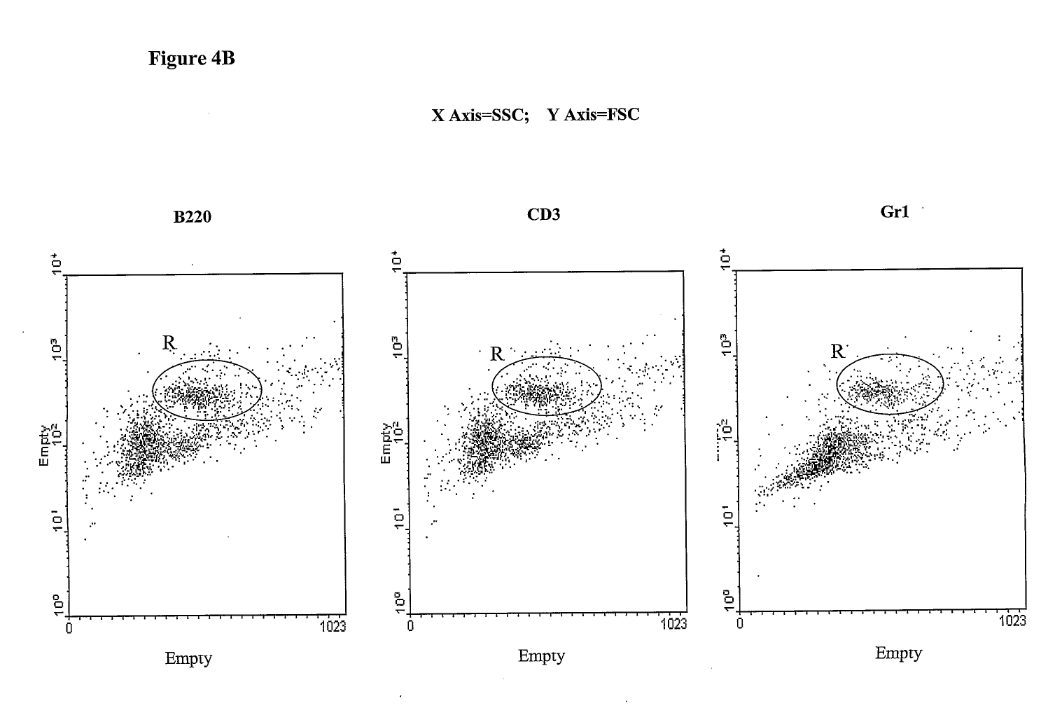

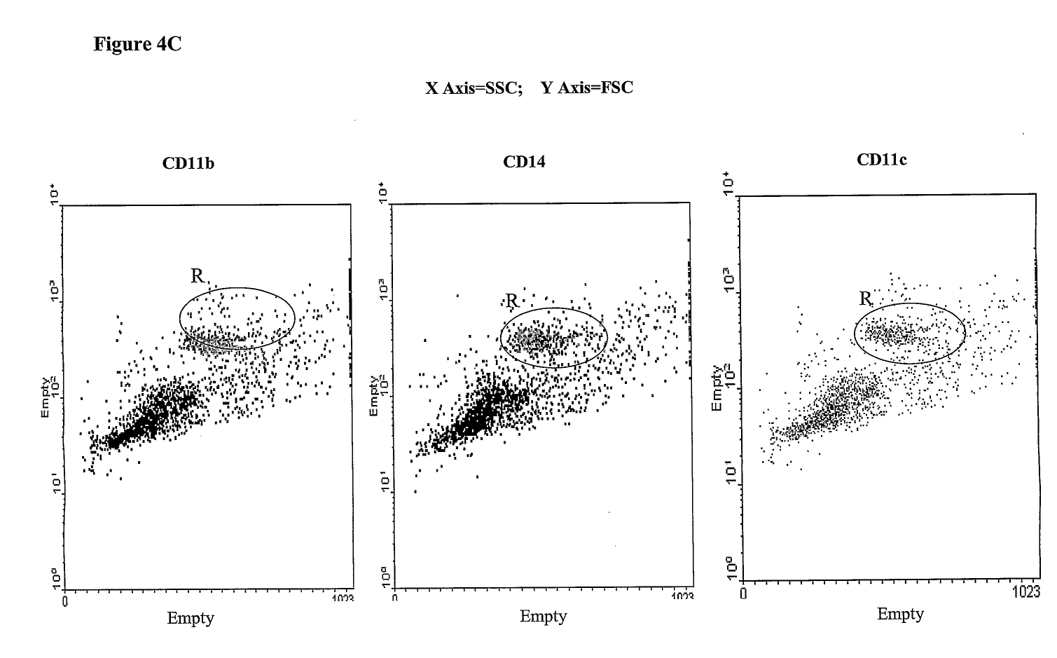

As shown in FIG. 3, Cy3-siRNA molecules were detected in 11-18% of whole BM cells 24 h following one injection (FIG. 3B) and in more than 30%, 24 h after two siRNA injections with 24 h intervals between injections (FIG. 3C) or 4 h following one siRNA bolus (FIG. 3A).

The Cy-3-positive BM cells were gated and further identified using antibodies directed to specific cell differentiation markers: Gr1, CD11b, CD11c and CD14 (Granulocytes, monocytes, macrophages, NK, DC and myeloid progenitor cells), B220 (B-cells), c-kit and SCA-1 (hematopoietic stem cells and progenitor cells), TER119 (Erythroid cells), Lin (T and B lymphocytes, monocytes, macrophages, NK cells, erythrocytes and granulocytes). FIG. 4 shows position of various antigen-positive cells on SSC/FSC plots. As shown, the subpopulation that is specifically targeted by siRNA is located in Region 1 (R1) that represents the majority of Gr1-, CD11c- and CD14-positive cells.

The percentage of Cy3-positive and Cy3-negative cells in each BM cell population was analyzed as follows:

### Lin/SCA1

The majority of cells in BM are Lin positive (90-94%). Approximately, ˜95% of Cy3-siRNA-positive cells are Lin positive cells.
Less then 1% of the progenitor SCA1+/Lin− showed siRNA uptake. (SCA1+/Lin-subpopulation constitute 1.4% out of whole BM cells)

### TER119/SCA1

Approximately 56% of the entire BM cells are TER119-positive cells and 12.5% out of them are progenitor cells expressing SCA1 cells. It appears that 24 h following siRNA injection, 33-45% of the Cy3-positive cells are TER119 positive (which are 2 fold higher compared to 4 h after siRNA injection). The increase in Cy3-siRNA positive cells expressing TER119 especially observed in the mature TER119 cells which are negative to SCA1.

### B220/CD3/SCA1

B220 positive cells constitute ˜15% of the entire BM cells.
   detection of siRNA in this population reached to 12% 24 h after 2 siRNA ionjections, however this population constitute only 2% out of the entire cells in the BM.
Approximately 20% of Cy3-siRNA positive cells expressing SCA1, only 1% of them are progenitor B cells (B220+/SCA1+).
CD3 positive cells constitute ˜9% of the entire BM cells. siRNA-uptake was obtained in no more than 10% CD3 cells which constitute less than 1% of the entire cells in the BM.ckit/SCA1/Gr1
SCA1 positive cells constitute ˜20% of the entire BM cells, approximately 20-24% of them are Cy3-positive (4% out of entire BM cells).
Ckit positive cells constitute 5% of the entire BM cells and only 1-2% of them were detected by Cy3 siRNA.
13% of SCA1 positive cells, are positive to Gr1 (SCA1+Gr1+) and they constitute 5% out of the entire cells in the BM. 24 h after 2 siRNA injections, 37% of SCA1+/Gr1+ were positive to Cy3.
   CD11b/Cd11c/Gr1
Approximately 38% of the entire BM cells are CD11b-positive cells and ˜33% are CD11c positive. 8% out of them are positive to both markers.
The majority of siRNA uptake was observed in CD11c−/CD11b+/Gr1+ population (myeloid progenitor cells, granulocyte, myeloid cells, myeloid DC, Macrophages). This population constitutes approximately 25% of the entire cell population in the BM and approximately 70% of them were Cy3-positive (8% out of total).
siRNA was also detected in CD11c+/CD11b−/Gr1− population (Lymphoid DC). Approximately 30-55% of them were Cy3 positive (7-14% out of total).

### CD14

CD14-CD11c+/Gr1− cells constitute 28% of the entire cells in the BM. 40-50% of these cells were found to be Cy3 positive cells (˜15% out of total). No evidence for Cy3 positive cells was shown in peripheral blood white blood cells.

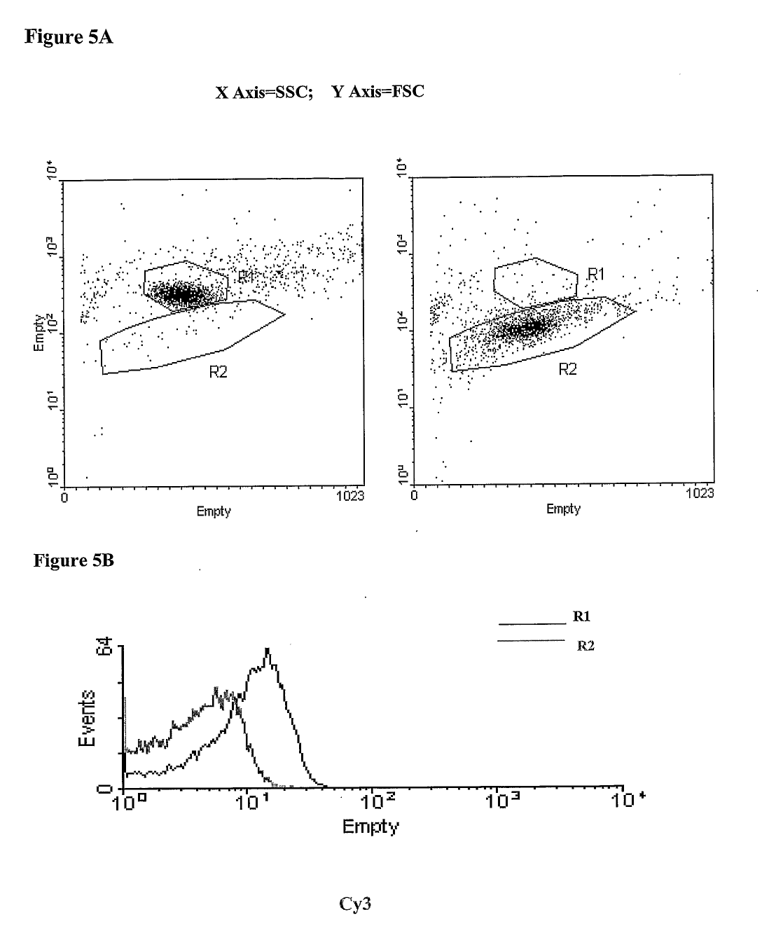

To better understand which cell population contains the highest level of Cy3-siRNA, Cy3-labeled siRNA was injected intraperitoneally (i.p.) to mice at 72 mg/kg dose. 4 h following injection, mice were sacrificed and BM cells, flushed out from the bones, were sorted to two sub populations based on their FSC/SSC parameters. FIG. 5 shows that the majority of R1 population is Cy3 positive while almost no detection of Cy3 was obtained in R2 population.

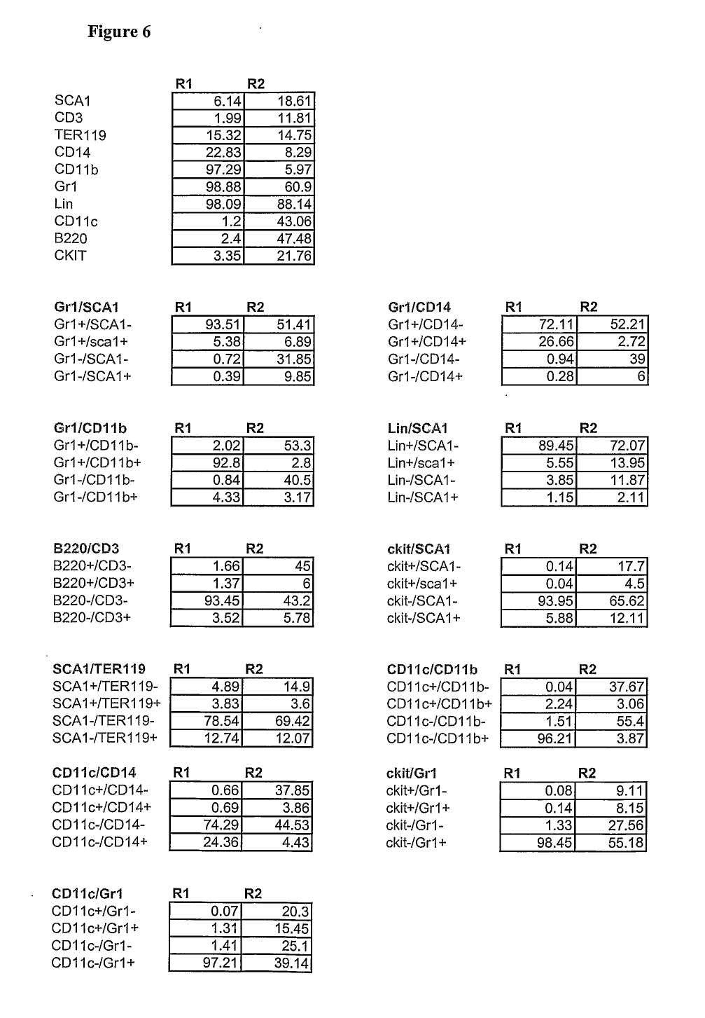

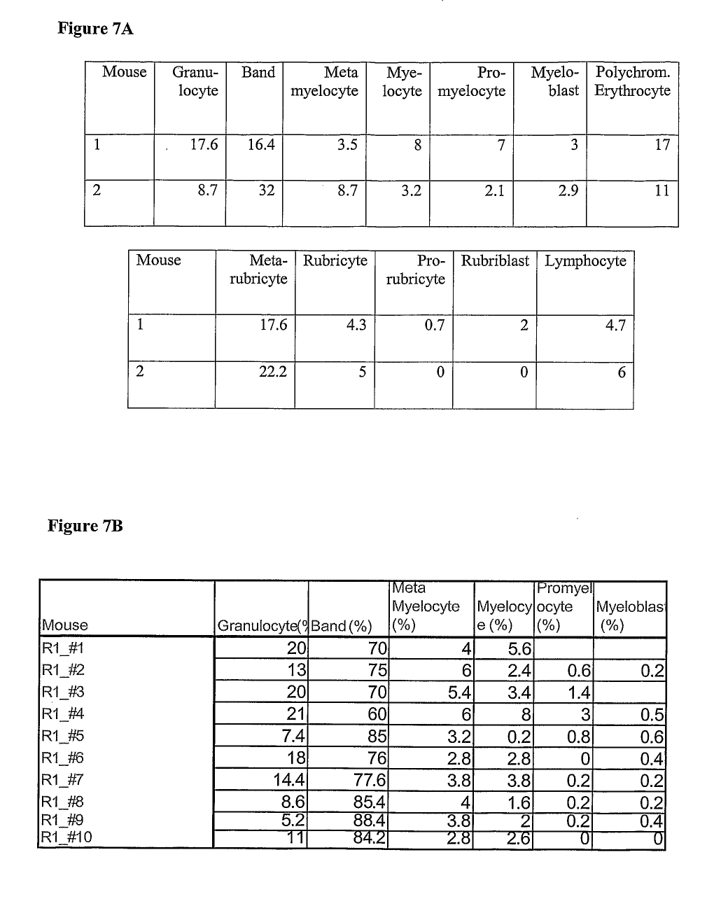

The sorted populations were identified using antibodies directed to specific cell differentiation markers or assessed by morphological features using Giemsa staining. As shown in FIG. 6, the majority of Cy3 positive cells (R1) are mature cells, expressing Gr1+/CD11b+ (92.8% cells in R1 compared to 1.4% cells in R2) and cells expressing Gr1+/CD14+ (26.6% cells in R1 compared to 1.38% cells in R2). Morphological inspection of R1 BM population after cells sorting and Giemsa staining showed enrichment of band population in relation to the whole BM cells (FIG. 7).

The same experiments are performed in human bone marrow cells using specific human bone marrow cell markers.

### Example 3

### Tumor-Bearing Mouse Model

Previous studies demonstrated an important role for suppression of antigen-specific T cell responses for Gr1+/CD11b+ cells in tumor-bearing hosts in tumor non-responsiveness. These cells may contribute to the failure of immune therapy in tumor-bearing mice and in patients with advanced stage cancer. Inoculation of transplantable tumor cells results in marked systemic expansion of Gr1+/CD11b+ cells in the bone marrow (BM), spleen and peripheral blood (PB). Therefore, based on the finding that siRNA is preferentially delivered to the Gr1+/CD11b+ cell population in the BM, a model of tumor bearing mice has been established in order to test siRNA delivery to those cells.

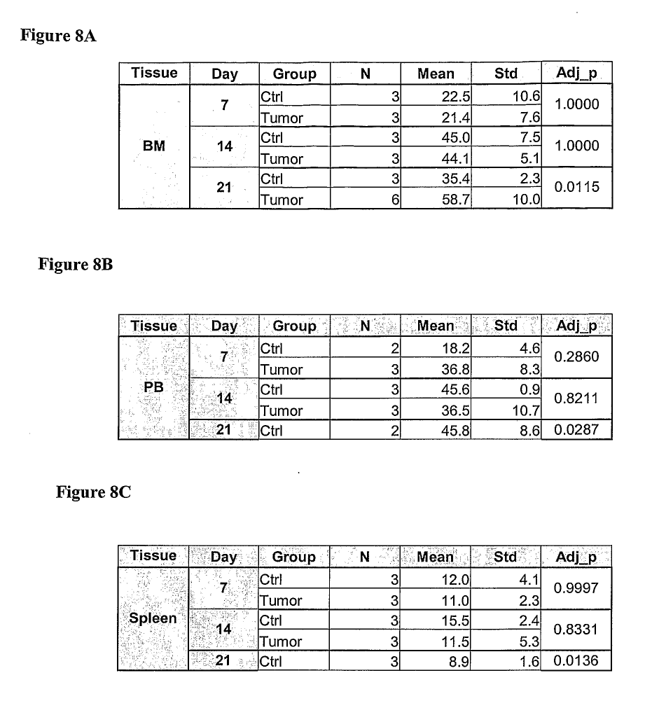

In mice bearing large metastatic Lewis lung carcinoma tumors (LLC1) the Gr1+/CD11b+ population in the BM, PB and spleen was assessed. Significant expansion of Gr1+/CD11b+ population in the BM, spleen and PB was observed 21 days after LLC1 cell transplantation (FIG. 8 A-C). Cy3-labeled siRNA was injected i.p. at a final dose of 72 mg/kg. About 24 h following injection, mice were sacrificed and detection of Cy3 siRNA was measured by FACS in the BM, PB, spleen and tumor. As positive control for siRNA delivery to the BM, a tumor-free mouse was also injected with Cy3-siRNA.

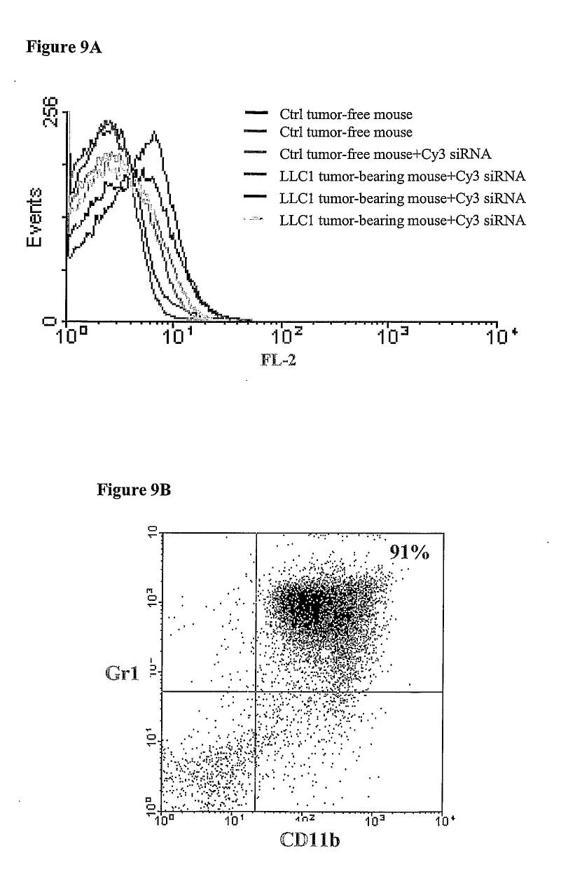

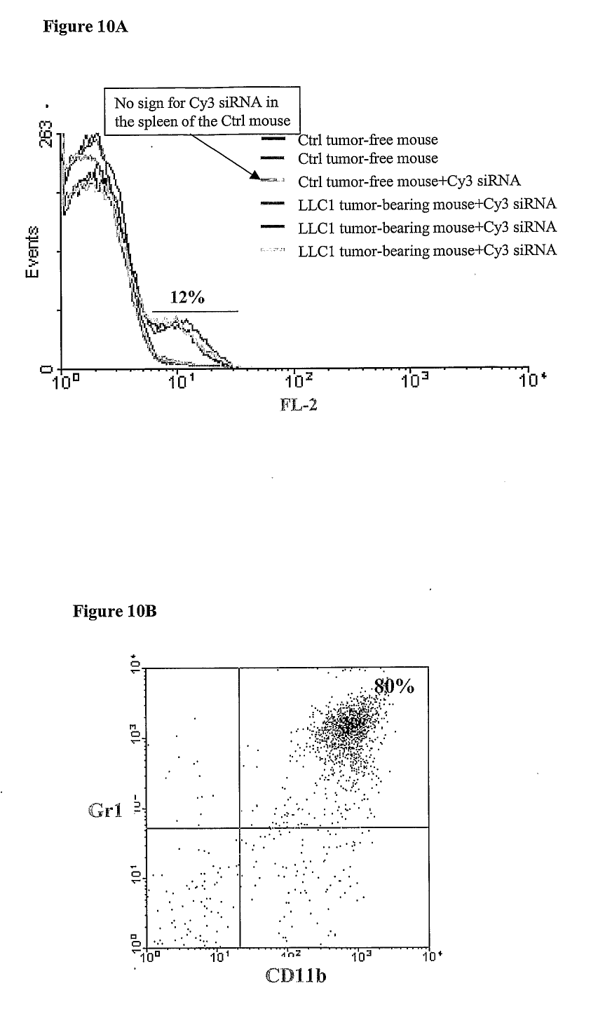

FIG. 9A shows detection of Cy3 siRNA in the BM of both groups (control and tumor-bearing mice). The majority of the siRNA-positive cells in the BM of tumor-bearing mice are Gr1+/CD11b+ cells (82-92%) (FIG. 9B). In the spleen of tumor-bearing mice, approximately, 12% of the cells were siRNA-positive while no sign of siRNA positive cells were found in the control mouse (FIG. 10A). Again, the majority of siRNA-positive cells were Gr1+/CD11b+ (65%-80) (FIG. 10B).

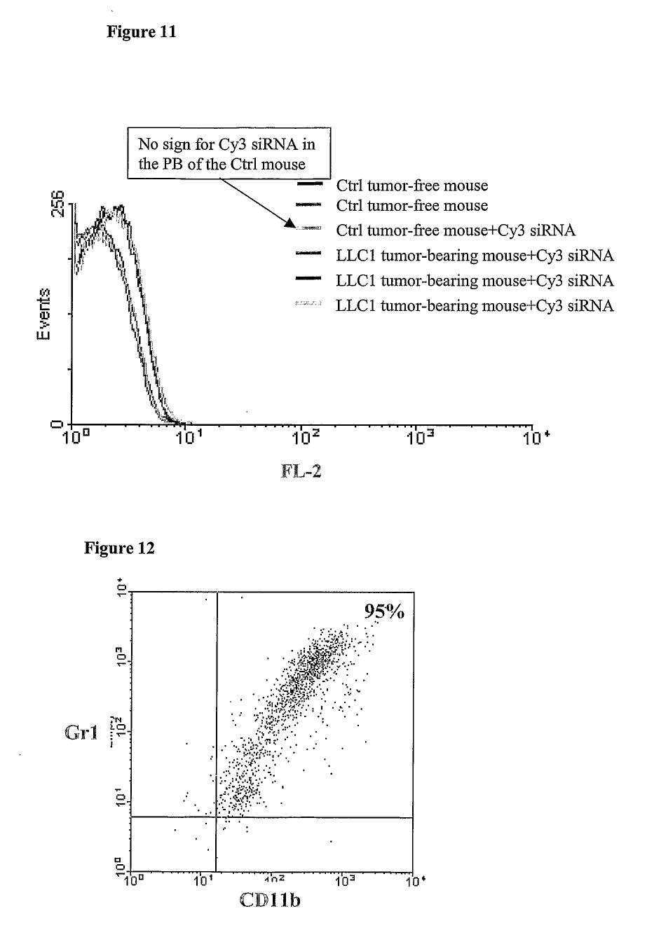

When the presence of the siRNA molecules in the PB was examined, the majority population in the PB of tumor-bearing mice was siRNA-positive, while no evidence of siRNA-positive cells was observed in the control mouse (FIG. 11).

In addition, siRNA-positive cells were found in the tumors. These cells were characterized by specific markers and found to be Gr1+/CD11b+ (90-95% of siRNA positive cells in the tumors) (FIG. 12). (In the same experiment siRNA was injected i.p. at a final dose of 72 mg/kg and the presence of siRNA in the tumor was measured by ISH 3 days after injection.) siRNA was identified in the tumor cells even three days after siRNA injection (data not shown).

These results demonstrated the delivery of siRNA to the Gr1+/CD11b+ population in mice and more importantly to those specific cells in tumor bearing mice.

### Example 4

### siRNA Delivery to Engrafted Human MonoMac1 Cells

To study the delivery of siRNA to human leukemic cells in the BM, non-obese diabetic/severe combined immunodeficiency (NOD/SCID) mice were used.

Methods: About 2×10⁷ human Monocytic leukemic cells (MonoMac1) were injected i.v. into sublethally irradiated NOD/SCID mice. 14 days after cell transplantation, approximately 45% engraftment of human MonoMac1 cells was observed. Cy3-siRNA was than injected i.p. at a dose of 72 mg/kg. After 4 h mice were sacrificed and the level of engraftment was estimated by immunofluorescent staining of CD45 on total BM cells.

CD45 positive cells were then gated and siRNA detection was measured by FACS using the FL-2 filter.

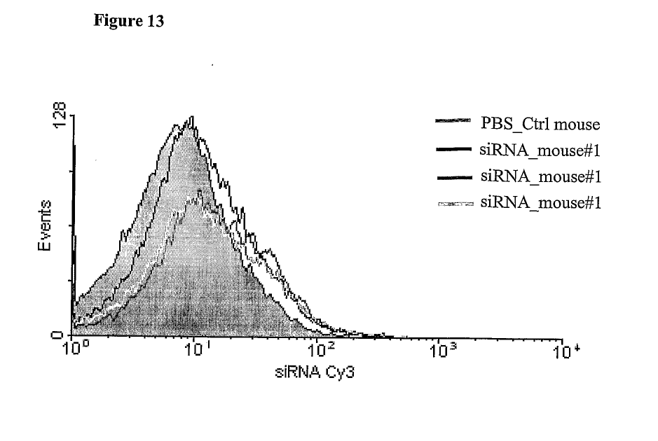

Results: As shown in FIG. 13, all the human Monomac1 cells were Cy3 positive (the whole CD45 population shifted in comparison to PBS injected mice). The shift in fluorescence is small but significant in all three injected mice.

### Example 5

### Animal Models for Allograft Transplant Rejection

One or more of the following animal models is used to test the siRNA compounds of the present invention for efficacy in treating allograft transplant rejection. Other animal models are also suitable. A model for corneal graft rejection: Kagaya et al., Exp Eye Res. 2002. 74(1):131-9. A model for cardiac graft rejection: Kim et al., Am J. Pathol. 2001. 158(3):977-86.

### Example 6

### Chemically Modified siRNA Activity as Tested in BM Cd11b Cells Isolated from Normal Mice

Casp2 was selected as a target gene in the bone marrow model. The CASP2_{—}4 sequence is disclosed in PCT Publication No. WO 2008/050329, assigned to the assignee of the present invention and hereby incorporated by reference in its entirety. The Casp2_{—}4 siRNA was chemically modified and tested for delivery to and activity in bone marrow cells. The chemically modified compounds are shown in Table C, below.

TABLE C
chemically modified CASP2_4 compounds

| ID | SiRNA compounds 5′->3′ |
|---|---|
| NP | GCCAGAAUGUGGAACUCCU |
| | AGGAGUUCCACAUUCUGGC |
| 45 | a-GCCAGAAUGUGGAAcUC-LC-U |
| | AGGAGUUCCACAUUCUGGC |
| 47 | C6-Im-Pi-GCCAGAAUGUGGAAcUC-LC-U |
| | AGGAGUUCCACAUUCUGGC |
| 90 | GCCAGAAUGUGGAACU-LC-LC-LT |
| | AGGAGUUCCACAUUCUGGC |
| 94 | GCCAGAAUGUGGAACUC-LC-U |
| | AGGAGUUCCACAUUCUGGC |
| 95 | GCCAGAAUGUGGAACU-LC-LC-U |
| | AGGAGUUCCACAUUCUGGC |

Key to Table C: Underlined: 2′OMe modified; small case italicized: DNA; C6-Im-Pi
= C6-Imino-Pi; a: abasic deoxyribo-pseudo-nucleotide; LC: L-deoxyribocytidine, LT:
1_deoxyribothymine

Methods: BM cells from C57BL/6 mice (male 8-10 weeks old) flushed out from the BM cavity of two femoral and two tibial bones. Epiphysial tissue was removed.

Bone marrow cavity of 2 femur and 2 tibia were flushed with 8 ml DMEM containing 20% fetal calf serum (FCS). Cells were filtered through a 50 μm mesh and precipitated at 1700 rpm for 10 min 4° C. Cells were then pooled, counted and purified based on their CD11b expression using CD11b (Mac-1) MicroBeads (Cat#130-049-601, Miltenyi Biotec). Cells from each mouse were dissolved in 90 ul bead buffer. Ten microliter (10ul) CD11b beads were then added to each sample (isolated from one mouse). The two samples (represents two mice) were combined and loaded on one magnetic column. Cells were collected and plated in 24 wells plate at final concentration of 1×10⁶ cells per 0.8 ml DMEM containing 20% serum.

CASP2_{—}4 siRNA compounds, at final concentration of 500 nM, 250 nM, were added to each well. RNA was extracted from CD11b cells 24 and 48 h after siRNA treatment. siRNA activity was tested by CASP2 gene expression by qPCR.

As shown in Table D, CASP2_{—}4 siRNA treatment leads to reduction in CASP2 gene expression in dose and time dependent manner. Results are shown as residual (% of Ctrl siRNA untreated cells) CASP2 expression

TABLE D
Mouse Casp2 expression as tested in BM purified CD11b+
cells 24 h and 48 h following siRNA treatment.

| | |
|---|---|
| 24 h following | 48 h following |
| siRNA treatment | siRNA treatment |

| siRNA | 250 nM | 500 nM | 250 nM | 500 nM |
|---|---|---|---|---|
| ID | siRNA | siRNA | siRNA | siRNA |
| NP | 60% | 41% | 66% | 44% |
| 45 | 65% | 61% | 45% | 32% |
| 47 | 50% | 66% | 51% | 32% |
| 90 | | 74% | 56% | 19% |
| 94 | 59% | 39% | 35% | 27% |
| 95 | 72% | 57% | 72% | 52% |

### Example 7

### siRNA Delivery to Human Engrafted Umbilical Cord Blood-Derived Cells in NOD/SCID Mice

Xenotransplantation systems are useful in initiating and maintaining the hematopoietic system in vivo. The nonobese diabetic/severe combined immuno-deficiency (NOD/SCID) mouse has been a useful model as a recipient for human BM cells growth. Cells from BM (Bone Marrow), PB (Peripheral Blood) and CB (Cord Blood) are used in in-vivo models for repopulating the BM of NOD/SCID mice (Guenechea et al., Blood. 1999, 93:1097-105; Glimm H et al., J Clin Invest. 2001, 107:199-206; Shultz L D, et al., J. Immunol. 1995, 154:180-91). Transplantation of human umbilical CB results in the engraftment of primitive cells that proliferate and differentiate to multiple lineages in the chimeric BM mouse (Bhatia M, et al. PNAS USA. 1997, 94:5320-5).

Human mononuclear cells (MNC's) were obtained from a CB specimen by density gradient centrifugation using Ficoll-Paque. Recipient NOD/SCID mice (NOD/LtSzPrKdc^{scid}/PrKdc^{scid}) were irradiated with a sublethal dose (350 cGy) from a cesium source 24 h before intravenously injection of the purified MNC (2×10⁷). Six weeks later BM cells flushed from both femur and tibia bones were harvested and resuspended into single-cell suspension. The percentage of human cells was determined by FACS following immunostaining with anti-human CD45-FITC Mab.

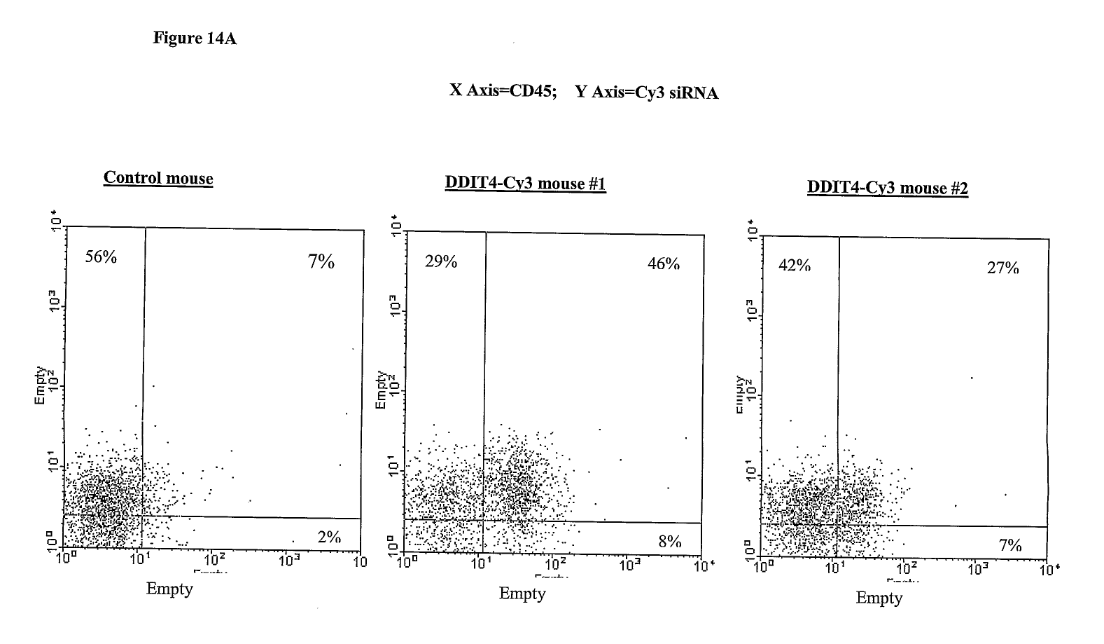

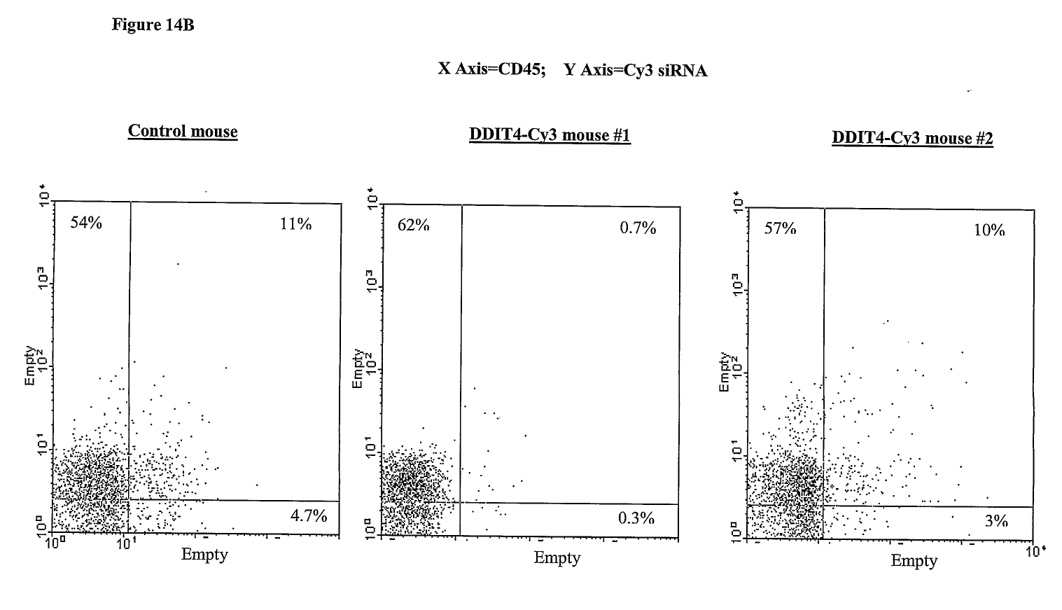

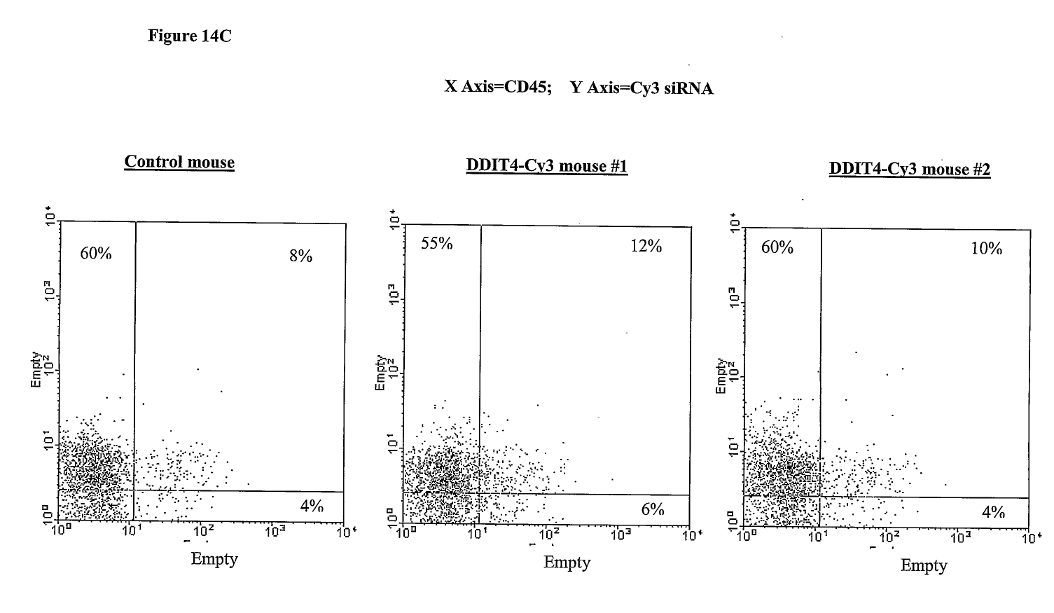

According to the yield of engraftment (80% human positive cells), Cy3 labeled siRNA was injected intraperitoneally (i.p) at final dose of 72 mg/kg to two chimeric mice. Chimeric mice were sacrificed 24 h following siRNA injection, and siRNA delivery to human cells in the BM, PB and Spleen was measured by FACS using FL-2 filter. The percentage of human cells in the BM of Cy3 siRNA injected chimeric mice (mouse #1 and #2) was 75% and 69%, respectively. Cy3 siRNA was detected in 69% and 39% out of the human engrafted cells, respectively (FIG. 14A). No sign of Cy3-siRNA positive cells was observed in the CD45 positive cells in the PB (FIG. 14B) or Spleen (FIG. 14C) of the chimeric mice.

The distribution of siRNA in BM human cells was analyzed by FACS following immunostaining with specific Abs directed to CD11b, CD33, CD14, CD10

Results: Tables E1-E3 show percentage of Cy3-siRNA positive human BM cells by their surface markers. The presence of Cy3 labeled siRNA was also tested in CD34−/CD38+ (Hematopoietic Stem Cells) or in progenitor stem cells CD34+/CD38+ but only part of these cells was found to be positive for siRNA.

TABLE E1

| | CD33+/CD11b+/ | CD33+/CD45+/ | CD11b+/CD45+/ |
|---|---|---|---|
| | CD45+ | siRNA+ | siRNA+ |
| Mouse | 32% | 16% (7% out of 23% | 33% (10% of human |
| #2 | | CD33+ cells | CD11b+ cells |
| | | were siRNA−) | were siRNA−) |
| Mouse | 23% | 34% (Only 50% | 19% (5% of human |
| #1 | | of CD33+ cells | CD11b+ cells |
| | | were siRNA+) | siRNA−) |

TABLE E2

| | CD33+/CD14+/CD45+ | CD14+/CD45+/siRNA+ |
|---|---|---|
| Mouse #2 | 11% | 63% of CD14 were siRNA+ |
| Mouse #1 | 17% | 50% of CD14 were siRNA+ |

TABLE E3

| | CD10+/CD45+ | CD10+CD45+/siRNA+ |
|---|---|---|
| Mouse #2 | 43% | 72% of CD10 were siRNA+ |
| Mouse #1 | 36% | 41% of CD10 were siRNA+ |

### Example 8

### CD11b+/Gr1+ Cell Expansion in Tumor Bearing NOD/SCID Mice

The majority of systemically delivered siRNA compounds target CD11b+/Gr1+BM cells of normal and tumor bearing mice and approximately 90% of human CD11b+ cells. A NOD/SCID tumor bearing chimeric mice model was established to test the (i) efficacy of the siRNA to reach the target cells and (ii) activity of siRNA in the target cells.

Methods: Expansion of CD11b+/Gr1+ cells in NOD/SCID-bearing human tumor cells was tested. NOD.Cg-Prkdc^{scid}B2m^{tm1Unc}/J, (NOD/SCID/2 null) mice were injected subcutaneously (s.c) with 5×10⁵ human HCT116 tumor cells or PC-3 prostate tumor cells. Approximately two months later when HCT116 tumor volume was 1 cm³ the mice were sacrificed and the CD11b+Gr1+ cells were quantified in the BM and the spleen (PC-3 injected mice do not developed tumors).

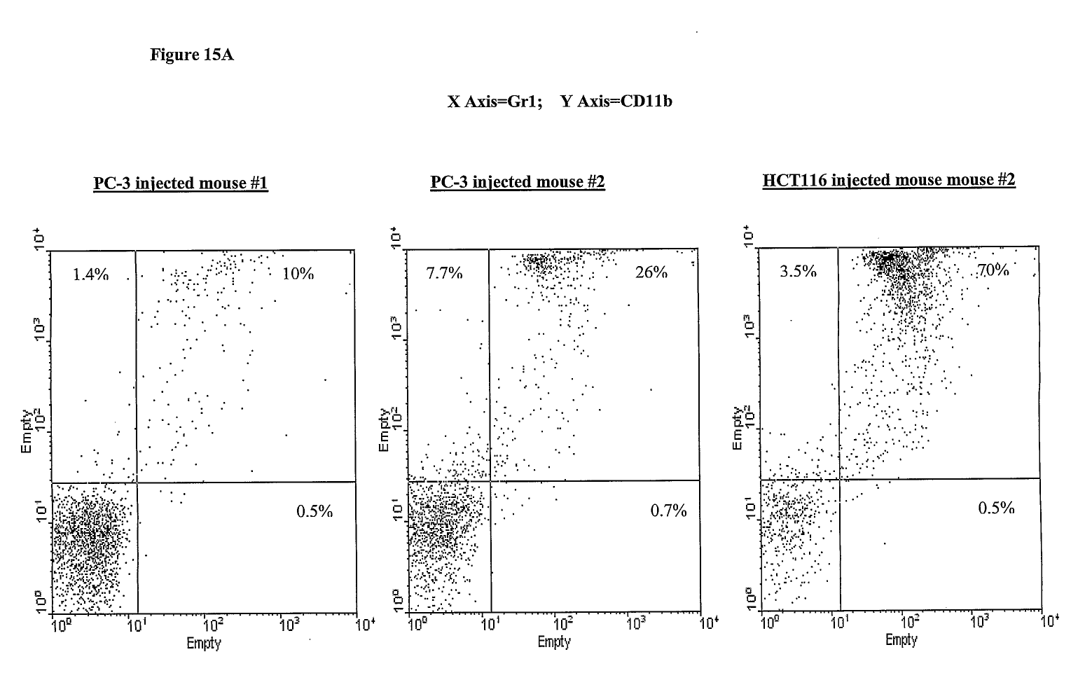

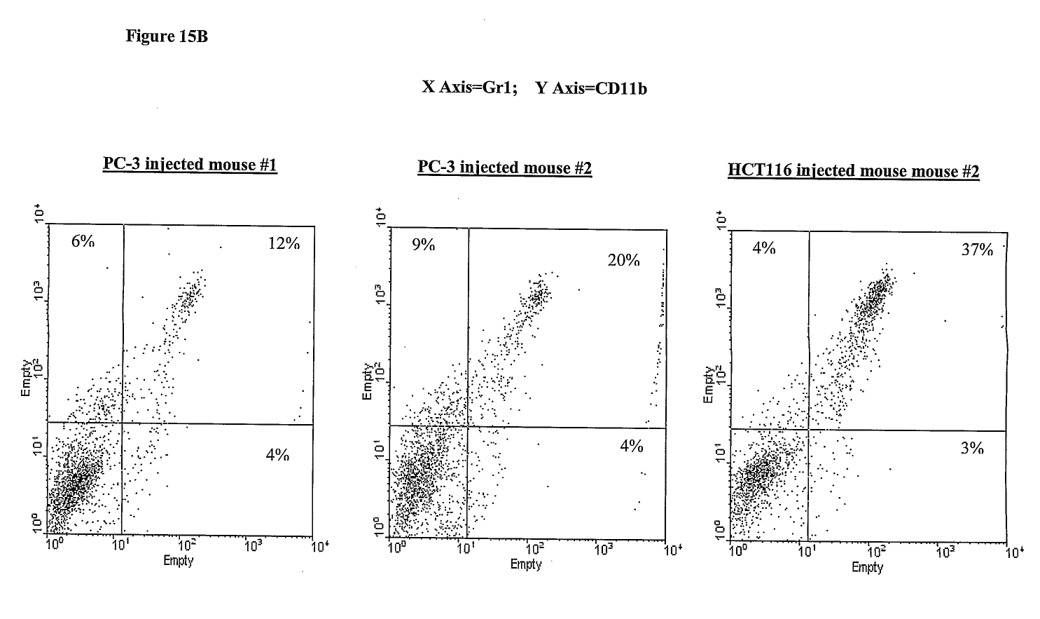

Results: FIGS. 15A-15B show CD11b+/Gr1+ cells expansion in the BM (15A) and the spleen (15B) of NOD/SCID/2 null mouse-bearing HCT116 tumor cells.

About 70% and 37% are CD11b+Gr1+ cells in the BM and the spleen of mice bearing HCT116 cells, respectively compared to 10-26% and 12-20% CD11b+/Gr1+ cells in the BM and the Spleen, respectively of mice injected with human PC-3 tumor cells.

### Example 9

### Gene Profile of Cd11b Cells from the BM and the Tumor of LLC1-TBM

Target gene expression level in a LLC1-TBM model was analyzed.

Methods: Approximately 9-10 week old C57BL/6 mice were injected s.c with LLC1 (Lewis lung carcinoma) cells. At days 14, 16, 18 and 21 mice were sacrificed and CD11b+ cells were purified from the BM and the tumor tissue using CD11b (Mac-1) MicroBeads. The expression level of CD80, MMP9, TGFb, PROK2, Arg1 and NOS2A was tested by qPCR at each time point.

Results: The gene expression results of BM CD11b+ cells were compared to those observed from the same cells isolated from Ctrl normal mice while the expression level of the tested gene in the tumor are presented as absolute number (expression of gene candidate normalized to reference gene).

As shown Table F1, there is significant induction in BM CD11b+ cells of CD80 (1.5-1.7 fold at days 14-16 and ×9 fold at day 21), MMP9 (1.5-1.9 fold induction), TGFb (induction of 2 fold at day 14), PROK2 (approximately 4-7.5 fold induction) and NOS2A genes (approximately 10-60 fold induction) compared to Ctrl cells.

TABLE F1
Gene expression in BM purified CD11b cells (n = 3)

| | CD80 | MMP9 | TGFb | PROK2 | ARG1 | NOS2 |
|---|---|---|---|---|---|---|
| Ctrl_Cd11b+ | 1.00 | 1.00 | 1.00 | 1.00 | 1.00 | 1.00 |
| _Normal mice | | | | | | |
| BM_Cd11b+ | 1.70 | 1.98 | 2.08 | 3.89 | 0.63 | 10.00 |
| 14 d | | | | | | |
| BM_Cd11b+ | 1.57 | 1.79 | 1.34 | 6.99 | 0.37 | 2.95 |
| 16 d | | | | | | |
| BM_Cd11b+ | 0.57 | 0.75 | 0.57 | 4.11 | 0.09 | 13.71 |
| 18 d | | | | | | |
| BM_Cd11b+ | 9.25 | 1.59 | 0.94 | 7.65 | 0.26 | 64.53 |
| 21 d | | | | | | |

A similar trend was observed for CD11b+ cells from the LLC1 tumor. Table F2 shows that CD80, MMP9 and PROK2 were induced from day 18, NOS2A was induced already at day 16 and TGFb was induced only at day 18. Induction of Arg1 expression was seen in tumor-CD11b cells but not in BM-CD11b cells.

TABLE F2
Gene expression in tumor-CD11b cells

| | CD80 | MMP9 | TGFb | PROK2 | ARG1 | NOS2 |
|---|---|---|---|---|---|---|
| T_Cd11b+ 14 d | 0.44 | 0.01 | 0.22 | 0.01 | | 0.09 |
| T_Cd11b+ 16 d | 0.37 | 0.01 | 0.21 | 0.00 | 0.10 | 0.15 |
| T_Cd11b+ 18 d | 0.77 | 0.04 | 1.20 | 0.02 | 0.18 | 0.09 |
| T_Cd11b+ 21 d | 2.75 | 0.04 | 0.76 | 0.09 | 0.20 | 0.44 |

### Example 10

### siRNA Directed to Partial List of the Targeted Genes

Some siRNA compounds targeting CD80 (SEQ ID NOS:24,076-24,087), TGFβ1 (SEQ ID NOS:24,088-24,095), Arg1 (SEQ ID NOS:24,96-24,099), NOS2A (SEQ ID NOS:24,100-24,103) and PROK2 (SEQ ID NOS:24, 104-24117) are shown in Table G.

TABLE G
siRNA to CD80, TGFβ, Arg1, NOS2A and PROK2

| | sense | antisense |
|---|---|---|
| si_ID | sequence 5′-3′ | sequence 5′-3′ |
| CD80_10 | GAGAACUAUCCAAAACUAA | UUAGUUUUGGAUAGUUCUC |
| CD80_11 | GGAGGUGACCCGAAUUAUA | UAUAAUUCGGGUCACCUCC |
| CD80_12 | GCAGUAAGCUAUCUUCAAA | UUUGAAGAUAGCUUACUGC |
| CD80_13 | CAGAGAGGUCUAACACCAA | UUGGUGUUAGACCUCUCUG |
| CD80_14 | GAGACUAUCUGAUUUCCUA | UAGGAAAUCAGAUAGUCUC |
| CD80_15 | GAUCGUUGUUUACAGUGUA | UACACUGUAAACAACGAUC |
| TGFb1_12 | CCUACAUUUGGAGCCUGGA | UCCAGGCUCCAAAUGUAGG |
| TGFb1_13 | CGGCAGCUGUACAUUGACU | AGUCAAUGUACAGCUGCCG |
| TGFb1_14 | GGCAGCUGUACAUUGACUU | AAGUCAAUGUACAGCUGCC |
| TGFb1_15 | CACACAGCAUAUAUAUGUU | AACAUAUAUAUGCUGUGUG |
| Arg1_1 | CCUUUCAAAUUGUGAAGAA | UUCUUCACAAUUUGAAAGG |
| Arg1_2 | GUCUCUACAUCACAGAAGA | UCUUCUGUGAUGUAGAGAC |
| NOS2A_1 | CAUAGUUUCCAGAAGCAGA | UCUGCUUCUGGAAACUAUG |
| NOS2A_2 | GCGCCUUUGCUCAUGACAU | AUGUCAUGAGCAAAGGCGC |
| PROK2_9 | GGGUCAAGAGCAUAAGGAU | AUCCUUAUGCUCUUGACCC |
| PROK2_10 | CUAGAAAAUGUCACUUGAA | UUCAAGUGACAUUUUCUAG |
| PROK2_11 | GCCACAUCUUACCUGUAAA | UUUACAGGUAAGAUGUGGC |
| PROK2_12 | CAAAAGUAAUCGCUCUGGA | UCCAGAGCGAUUACUUUUG |
| PROK2_13 | CUGUCAGUAUCUGGGUCAA | UUGACCCAGAUACUGACAG |
| PROK2_14 | CCAUCCACUGACUCGUAAA | UUUACGAGUCAGUGGAUGG |
| PROK2_15 | CCUUAGUCUCCUACUUAGA | UCUAAGUAGGAGACUAAGG |

Transfection protocol for PROK2 siRNA: Approximately 2×10⁵ human Saos2 cells (human osteosarcoma) expressing the endogenous PROK2 gene were seeded per well in 6 wells plate (70-80% confluent).

Cells were transfected with siRNA oligos 24 h later using Lipofectamine 2000™ reagent (Invitrogene) according manufacturer's procedure, at final concentration of 500 pM, 5 nM and 20 nM. Cells were harvested 72 h after transfection, RNA was extracted from cells and PROK2 gene expression was tested by qPCR. Activity is presented as residual (% of Ctrl) human PROK2 expression in Saos2 cells (Table H).

TABLE H
PROK2 expression in Saos2 cells as tested
by qPCR following siRNA transfection.

| | | | |
|---|---|---|---|
| Ctrl | | | 100 |
| PROK2_9 | 20 | nM | 11 |
| | 5 | nM | 18 |
| | 0.5 | nM | 73 |
| PROK2_10 | 20 | nM | 28 |
| | 5 | nM | 40 |
| | 0.5 | nM | |
| PROK2_11 | 20 | nM | 5 |
| | 5 | nM | 21 |
| | 0.5 | nM | 67 |
| PROK2_12 | 20 | nM | 28 |
| | 5 | nM | 19 |
| | 0.5 | nM | |
| PROK2_13 | 20 | nM | 20 |
| | 5 | nm | 48 |
| | 0.5 | nM | |
| PROK2_14 | 20 | nM | 6 |
| | 5 | nm | 20 |
| | 0.5 | nM | 81 |
| PROK2_15 | 20 | nM | 47 |
| | 5 | nm | 32 |
| | 0.5 | nM | |

### Example 11

### Cy3-siRNA Positive Tumor CD11b Cells

Example 3 hereinabove demonstrates (by FACS) that the majority of Cy3 positive cells following administration of Cy3-siRNA are CD11b+Gr1+ cells (FIG. 12). In the present experiment CD11b+ cells were purified from the tumor and the presence of siRNA in these cells was shown by confocal microscopy.

Methods: C57BL/6 mice (9-10 weeks old) were injected s.c with LLC1 cells, 20 days later, Cy3-labeled siRNA oligo was injected i.p at final dose of 72 mg/kg. 24 h after siRNA injection, CD11b+ cells were purified from the tumor tissue using CD11b (Mac-1) Microbeads. Cells were than stained by FITC-conjugated CD11b and PerCP-Cy5.5-conjugated Gr1 Abs to determine yield of purification. After staining, cells were fixed with 4% PFA and presence of siRNA was observed by confocal microscopy.

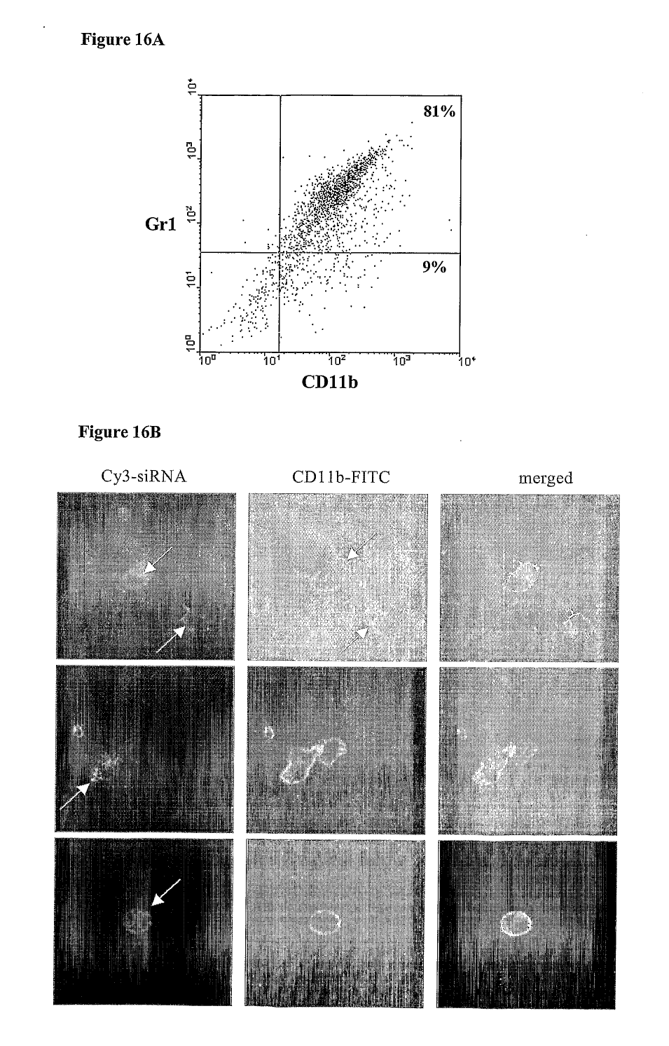

FIG. 16A shows that the yield of CD11b+/Gr1+ purified cells was 81% and yield of CD11b+/Gr1− purified cells was 9%.

FIG. 16B shows siRNA detection in CD11b+ cells from the tumor tissue by Confocal microscopy. Arrows show some of the labeled siRNA (left most figures) and labeled cells (center figures). The right-most figures show merging of the siRNA and cells.

### Example 12

### Microarray Data—Gene Profile of BM and Tumor Cd11b+/Gr1+Cells from Mice Bearing Human Tumors

Microarray analysis is performed to identify gene expression pattern in the CD11b+/CD33+ cells and the associated tumors.

1. A method of treating a disorder associated with immature myeloid cell expansion and/or mobilization in a subject in need of such treatment which comprises systemically administering to the subject a therapeutically effective amount of an siRNA directed to a target gene associated with the disorder in an amount effective to treat the subject.

2. The method of claim 1, wherein the immature myeloid cell is a CD33+/CD11b+ cell.

3. The method of claim 1, wherein the disorder associated with immature myeloid cell expansion and/or mobilization is tumorigenesis, tumor progression, tumor neoangiogenesis, tumor resistance, or allograft rejection.

4. (canceled)

5. The method of claim 3, wherein the disorder is a solid tumor, a hematopoietic tumor of a myeloid lineage or a head and neck, breast, lung, kidney, prostate, colon or pancreatic tumor.

6-7. (canceled)

8. The method of claim 1 further comprising administering a chemotherapeutic agent to the subject.

9. The method of claim 3, wherein disorder is an allograft rejection.

10. The method of claim 1, wherein the siRNA is a chemically modified siRNA.

11. The method of claim 10, wherein the chemically modified siRNA is chemically modified according to any one of structural motifs (A)-(P).

12. (canceled)

13. The method of claim 1, wherein the siRNA is a naked siRNA.

14. The method of claim 1, wherein the target gene is selected from the group consisting of genes for which the corresponding mRNA has a sequence set forth in any one of SEQ ID NOS: 1-86.

15. The method of claim 1, wherein the target gene is CD80, MMP9, PROK2, NOS2A, ARG1, TGFβ2, or CD86.

16. (canceled)

17. The method of claim 1, wherein the siRNA comprises an antisense sequence present in Tables B (1-B25; SEQ ID NO:90-24,075) or Table G (SEQ ID NO:24,076-24,117).

18. A method of treating a cancer in a subject in need thereof which comprises systemically administering to the subject a therapeutically effective amount of an oligonucleotide which inhibits expression of a target gene expressed in an immature myeloid cell in the subject in an amount effective to treat the subject.

19. A method of reducing immature myeloid cell expansion or mobilization in a subject in need thereof which comprises systemically administering to the subject a therapeutically effective amount of an oligonucleotide which inhibits expression of a target gene expressed in the immature myeloid cell in an amount effective to reduce immature myeloid cell expansion or mobilization.

20. A method of treating a subject suffering from a disorder in which the level of T cell receptor zeta chain (CD3ζ) is reduced or absent which comprises systemically administering to the subject an oligonucleotide which inhibits expression of a target gene expressed in an immature myeloid cell in the subject in an amount effective to treat the subject.

21. A method of reducing tumor vascularization and tumor progression in a subject in need thereof which comprises systemically administering to the subject a therapeutically effective amount of an oligonucleotide which inhibits expression of a target gene expressed in an immature myeloid cell in the subject in an amount effective to reduce tumor vascularization and tumor progression.

22. A method of preventing transplant rejection in a subject in need thereof which comprises systemically administering to the subject a therapeutically effective amount of an oligonucleotide which inhibits expression of a target gene expressed in an immature myeloid cell in the subject in an amount effective to treat the subject.

23. A method of delivering an oligonucleotide to a CD11b+ immature myeloid cell in a subject in need thereof which comprises administering systemically to the subject a therapeutically effective amount of an oligonucleotide which inhibits expression of a target gene expressed in the immature myeloid cell in an amount effective to achieve delivery to the CD11b+ immature myeloid cell.

24-32. (canceled)

33. A compound set forth as Structure (A):

| | |
|---|---|
| (A) | 5′ (N)ₓ-Z 3′ (antisense strand) |
| | 3′ Z′-(N′)_{y} 5′ (sense strand) |

wherein each of N and N′ is a nucleotide selected from an unmodified ribonucleotide, a modified ribonucleotide, an unmodified deoxyribonucleotide and a modified deoxyribonucleotide;
wherein each of (N)ₓ and (N′)_{y} is an oligonucleotide in which each consecutive N or N′ is joined to the next N or N′ by a covalent bond;
wherein each of x and y is an integer between 18 and 40;
wherein each of Z and Z′ may be present or absent, but if present is 1-5 consecutive nucleotides covalently attached at the 3′ terminus of the strand in which it is present; wherein the sequence of (N′)_{y} is present within an mRNA expressed in an immature myeloid cell; and wherein the sequence of the mRNA is set forth in Tables A1 and A2 (SEQ ID NOS:1-89).

34. A compound having Structure (I) set forth below:

| | |
|---|---|
| (I) | 5′ (N)x-Z 3′ (antisense strand) |
| | 3′ Z′-(N′)y-z″ 5′ (sense strand) |

wherein each of N and N′ is a ribonucleotide which may be unmodified or modified, or an unconventional moiety;
wherein each of (N)x and (N′)y is an oligonucleotide in which each consecutive N or N′ is joined to the next N or N′ by a covalent bond;
wherein Z and Z′ may be present or absent, but if present is independently 1-5 consecutive nucleotides covalently attached at the 3′ terminus of the strand in which it 5 is present;
wherein z″ may be present or absent, but if present is a capping moiety covalently attached at the 5′ terminus of (N′)y;
wherein each of x and y are independently 18 to 27;
wherein (N)x comprises modified and unmodified ribonucleotides, each modified ribonucleotide having a 2′-O-methyl on its sugar, wherein N at the 3′ terminus of (N)x is a modified ribonucleotide, (N)x comprises at least five alternating modified ribonucleotides beginning at the 3′ end and at least nine modified ribonucleotides in total and each remaining N is an unmodified ribonucleotide;
wherein in (N′)y at least one unconventional moiety is present, which unconventional moiety may be an abasic ribose moiety, an abasic deoxyribose moiety, a modified or unmodified deoxyribonucleotide, a mirror nucleotide, and a nucleotide joined to an adjacent nucleotide by a 2′-5′ internucleotide phosphate bond; and
wherein the sequence of (N)x is substantially complementary to the sequence of (N′)y; and the sequence of (N′)y is substantially identical to the sequence of an mRNA set forth in Tables A1 and A2 (SEQ ID NOS:1-89).

35-37. (canceled)

38. A method of treating a disorder associated with immature myeloid cell expansion and/or mobilization in a subject in need of such treatment which comprises systemically administering to the subject therapeutically effective amount of an siRNA according to claim 33 in an amount effective to treat the subject.

39. A method of treating a disorder associated with immature myeloid cell expansion and/or mobilization in a subject in need of such treatment which comprises systemically administering to the subject a therapeutically effective amount of an siRNA according to claim 34 in an amount effective to treat the subject.
